(19) United States
(12) Patent Application Publication
(10) Pub. No.: US 2026/0234722 A1
(43) Pub. Date: Aug. 13, 2026

(54) METHOD OF DETERMINING PROLIFERATION CAPACITY

(71) Applicant: THE UNIVERSITY OF LIVERPOOL, Liverpool Merseyside (GB)

(72) Inventor: Anthony HOLLANDER, Liverpool Merseyside (GB)

(73) Assignee: THE UNIVERSITY OF LIVERPOOL, Liverpool Merseyside (GB)

(21) Appl. No.: 19/471,829

(22) PCT Filed: Apr. 4, 2024
(86) PCT No.: PCT/GB2024/050915

§ 371 Date: Oct. 2, 2025

(30) Foreign Application Priority Data
Apr. 5, 2023 (GB) 2305070.1

(51) Int. Cl. C12Q 1/6876; A61K 35/28; C12N 5/0775

(52) U.S. Cl. CPC C12Q 1/6876; A61K 35/28; C12N 5/0663; C12Q 2600/158

(57) ABSTRACT

The present invention relates to a method of determining or identifying whether a mesenchymal stem cell has high proliferative capacity, a method of selecting said mesenchymal stem cells, a mesenchymal stem cell having high proliferative capacity, a pharmaceutical composition thereof, its method of preparation, and its use as a medicament.

### FIELD OF THE INVENTION

The present invention relates to a method of determining or identifying whether a mesenchymal stem cell has high proliferative capacity, a method of selecting said mesenchymal stem cells, a mesenchymal stem cell having high proliferative capacity, a pharmaceutical composition thereof, its method of preparation, and its use as a medicament.

### BACKGROUND TO THE INVENTION

Mesenchymal stem cells (MSCs) have been investigated as a potential injectable therapy for the treatment of knee osteoarthritis and other diseases, with some evidence of success in preliminary human trials¹.

If MSCs are to be used as donor cells to treat large numbers of patients, then any one single donor sample will need to be expanded through cell culture to create a Master Cell Bank from which batches of cells can be further expanded to create working cell banks for the production of the therapeutic product. However, while MSCs (derivable for example from bone marrow or adipose tissue) have the capacity to proliferate for multiple generations, eventually the rate of proliferation slows down and they become senescent and cease to grow. The effectiveness of any one donated bone marrow or adipose sample in generating large numbers of therapeutic product therefore depends on the rate of growth of the cells and the time taken to reach senescence.

Crucially, MSCs from different patients begin to grow at different rates after the first few passages and those which start to proliferate more slowly also tend to reach senescence much earlier¹. MSC proliferation capacity can therefore ultimately determine the amount of drug product that a bone marrow or adipose sample can generate, and this capacity can vary widely across different samples.

In order to render possible the use of MSCs as a form of therapy, it would be desirable to be able to select donor MSCs soon after the start of culture for their capacity to grow faster and for longer so that their trophic activity, and consequent therapeutic potential, can be fully exploited. Until now, it has not been possible to predict at the start of culture which donor cells will have these characteristics and therefore a high proliferation capacity. The inability to select cells with high proliferative capacity in turn limits the ability to generate large amounts of therapeutic product, thus limiting the overall efficiency of generating therapeutic product. One or more aspects or embodiments of the present invention seek to address at least this problem, or one or more alternative problems in the art.

### SUMMARY OF THE INVENTION

A first aspect of the invention provides a method of determining, identifying or selecting a mesenchymal stem cell (MSC) having a high proliferative capacity, the method comprising:
a. Obtaining data indicative of the level of expression of one or both of the following genes: GAS6 and GAS6-AS1 in the mesenchymal stem cell;
b. Optionally calculating a ratio of the level of gene expression of GAS6-AS1:GAS6;
c. Comparing the level of expression of one or both of the genes of step (a), and/or optionally the ratio of step (b), to a reference value;
d. Determining, identifying or selecting the mesenchymal stem cell having a high proliferative capacity if the level of expression of the GAS6 gene, GAS6-AS1 gene and/or the ratio of GAS6-AS1:GAS6 gene expression levels, is below the reference value.

A suitable embodiment of the first aspect of the invention provides a method of determining whether a mesenchymal stem cell has a high proliferative capacity, the method comprising:
a. Obtaining data indicative of the level of expression of one or both of the following genes: GAS6 and GAS6-AS1 in the mesenchymal stem cell;
b. Optionally calculating a ratio of the level of gene expression of GAS6-AS1:GAS6;
c. Comparing the level of expression of one or both of the genes of step (a), and/or optionally the ratio of step (b), to a reference value;
d. Determining that the mesenchymal stem cell has a high proliferative capacity if the level of expression of the GAS6 gene, GAS6-AS1 gene and/or the ratio of GAS6-AS1:GAS6 gene expression levels, is below the reference value.

A suitable embodiment of the first aspect of the invention provides a method of identifying/selecting a mesenchymal stem cell having a high proliferative capacity (optionally for therapeutic use), the method comprising:
a. Obtaining data indicative of the level of expression of one or both of the following genes: GAS6 and GAS6-AS1 in the mesenchymal stem cell;
b. Optionally calculating a ratio of the level of gene expression of GAS6-AS1:GAS6;
c. Comparing the level of expression of one or both of the genes of step (a), and/or optionally the ratio of step (b), to a reference value;
d. Selecting the mesenchymal stem cell if the level of expression of the GAS6 gene, GAS6-AS1 gene and/or the ratio of GAS6-AS1:GAS6 gene expression levels, is below the reference value.

A second aspect of the invention provides a method of distinguishing between a mesenchymal stem cell having a high proliferative capacity and a mesenchymal stem cell having a low proliferative capacity, the method comprising:
a. Obtaining data indicative of the level of expression of one or both of the following genes: GAS6 and GAS6-AS1 in a mesenchymal stem cell;
b. Optionally calculating a ratio of the level of gene expression of GAS6-AS1:GAS6;
c. Comparing the level of expression of one or both of the genes of step (a), and/or optionally the ratio of step (b), to a reference value;
d. Distinguishing the mesenchymal stem cell as (i) having a high proliferative capacity if the level of expression of the GAS6 gene, GAS6-AS1 gene and/or the ratio of GAS6-AS1:GAS6 gene expression levels, is below the reference value, or (ii) having a low proliferative capacity if the level of expression of the GAS6 gene, GAS6-AS1 gene and/or the ratio of GAS6-AS1:GAS6 gene expression, is above the reference value.

In one embodiment, the above methods are applied to a mesenchymal stem cell in a population of mesenchymal stem cells. Suitably therefore, referring to a method of determining, identifying or selecting a mesenchymal stem cell (MSC) having a high proliferative capacity from a population of mesenchymal stem cells, or to a method of distinguishing between a mesenchymal stem cell having a high proliferative capacity and a mesenchymal stem cell having a low proliferative capacity in a population of mesenchymal stem cells. In one embodiment, the above methods are applied to a plurality of mesenchymal stem cells, suitably to a population of mesenchymal stem cells.

A third aspect of the invention provides use of the level of expression of one or both of the following genes GAS6 and GAS6-AS1, and/or the ratio of gene expression levels of GAS6-AS1:GAS6, as an indicator of the proliferative capacity of a mesenchymal stem cell.

A fourth aspect of the invention provides a mesenchymal stem cell having a high proliferative capacity, wherein level of expression of the GAS6 gene, GAS6-AS1 gene and/or the ratio of GAS6-AS1:GAS6 gene expression levels in the mesenchymal stem cell is below a reference value.

In one embodiment, the mesenchymal stem cell is obtained from a method according to the first aspect.

In one embodiment the mesenchymal stem cell is isolated or selected, suitably from a population of mesenchymal stem cells.

A fifth aspect of the invention provides a population of mesenchymal stem cells according to the fourth aspect of the invention.

Cells of the fourth and fifth aspects of the invention may be referred to herein as “cells of the invention”, “mesenchymal cells of the invention”, or “MSCs of the invention”.

A sixth aspect of the invention provides a combination of a mesenchymal stem cell according to the fourth aspect of the invention, or a population of mesenchymal stem cells according to the fifth aspect of the invention, and a therapeutic agent.

A seventh aspect of the invention provides use of a mesenchymal stem cell according to the fourth aspect of the invention, or a population of mesenchymal stem cells according to the fifth aspect of the invention for the delivery of a therapeutic agent.

An eighth aspect of the invention provides a pharmaceutical composition comprising a population of mesenchymal stem cells according to the fifth aspect of the invention, or the combination according to the sixth aspect.

A ninth aspect of the invention provides a population of mesenchymal cells of the fifth aspect and/or a pharmaceutical composition of the eighth aspect and/or a combination of the sixth aspect for use as a medicament.

A tenth aspect of the present invention provides a population of mesenchymal cells of the fifth aspect and/or a pharmaceutical composition of the eighth aspect and/or a combination of the sixth aspect for use in the treatment of a bone or soft tissue disease or injury, a liver disease or injury, a bowel disease or injury, a lung disease or injury, a skin disease or injury, a heart disease or injury, a kidney disease or injury, a uterus disease or injury, or any combination thereof.

An eleventh aspect of the invention provides a method of preparing a population of mesenchymal stem cell for use as a medicament, the method comprising:
a. Culturing a population of mesenchymal stem cells;
b. Selecting those mesenchymal stem cells in which the level of expression of the GAS6 gene, GAS6-AS1 gene and/or the ratio of GAS6-AS1:GAS6 gene expression levels is below a reference value;
c. Optionally expanding the selected mesenchymal stem cells;
d. Formulating the mesenchymal stem cells for use as a medicament

A twelfth aspect of the invention provides a medicament prepared by the method of the eleventh aspect of the invention.

A thirteenth aspect of the invention provides a medicament prepared by the method of the eleventh aspect of the invention for use in the treatment of a bone or soft tissue disease or injury, a liver disease or injury, a bowel disease or injury, a lung disease or injury, a skin disease or injury, a heart disease or injury, a kidney disease or injury, a uterus disease or injury, or any combination thereof.

In a further aspect of the present invention there is provided a method of identifying or selecting a mesenchymal stem cell (MSC), the method comprising:
a. Obtaining data indicative of the level of expression of one or both of the following genes: GAS6 and GAS6-AS1 in the mesenchymal stem cell;
b. Optionally calculating a ratio of the level of gene expression of GAS6-AS1:GAS6;
c. Identifying or selecting the mesenchymal stem cell based upon the level of expression of GAS6, GAS6-AS1 and/or the ratio of the level of gene expression of GAS6-AS1:GAS6.

In some embodiments, the MSC is a desirable MSC having suitably desirable characteristics. For example, the desirable MSC may have a high proliferative capacity. In some embodiments, the MSC is identified or selected based on a desirable level of expression of GAS6, GAS6-AS1 and/or the ratio of the level of gene expression of GAS6-AS1:GAS6. Suitably a desirable level of expression may be a level of expression of GAS6, GAS6-AS1 and/or the ratio of the level of gene expression of GAS6-AS1:GAS6 which reflects a high proliferative capacity.

A further aspect of the invention provides a method of distinguishing between mesenchymal stem cells, the method comprising:
a. Obtaining data indicative of the level of expression of one or both of the following genes: GAS6 and GAS6-AS1 in a mesenchymal stem cell;
b. Optionally calculating a ratio of the level of gene expression of GAS6-AS1:GAS6;
c. Distinguishing the mesenchymal stem cell by the level of expression of the GAS6 gene, GAS6-AS1 gene and/or the ratio of GAS6-AS1:GAS6 gene expression levels.

In some embodiments the method is a method of distinguishing between desirable and undesirable mesenchymal stem cells. For example, the desirable MSC may have a high proliferative capacity, the undesirable MSC may have a low proliferative capacity. In some embodiments, the MSC is distinguished based on a desirable level of expression of GAS6, GAS6-AS1 and/or the ratio of the level of gene expression of GAS6-AS1:GAS6. Suitably a desirable level of expression may be a level of expression of GAS6, GAS6-AS1 and/or the ratio of the level of gene expression of GAS6-AS1:GAS6 which reflects a high proliferative capacity. In some embodiments, the MSC is distinguished based on an undesirable level of expression of GAS6, GAS6-AS1 and/or the ratio of the level of gene expression of GAS6-AS1:GAS6. Suitably an undesirable level of expression may be a level of expression of GAS6, GAS6-AS1 and/or the ratio of the level of gene expression of GAS6-AS1:GAS6 which reflects a low proliferative capacity. Optionally the method may further comprise a step of separating the MSC based on its of expression of the GAS6 gene, GAS6-AS1 gene and/or the ratio of GAS6-AS1:GAS6 gene expression levels. In one embodiment the method comprises obtaining data in a plurality of MSCs, and optionally calculating the ratio in a plurality of MSCs, and then distinguishing between the MSCs by the level of expression of the GAS6 gene, GAS6-AS1 gene and/or the ratio of GAS6-AS1:GAS6 gene expression levels.

### DETAILED DESCRIPTION OF THE INVENTION

Features and embodiments further to the above aspects will now be described under the following headed sections, however any feature, embodiment or definition described herein is not limited to any particular aspect, and may be combined with any aspect in any workable combination.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The terms “proliferation capacity” and “proliferative capacity” are used interchangeably herein unless the context explicitly indicates otherwise.

Surprisingly the inventors have discovered that an unusual gene and its antisense counterpart can be used to predict the likelihood that a mesenchymal stem cell or population thereof will have a high proliferative capacity i.e. will grow faster and for a longer duration of time than another MSC or population of MSCs. This builds on prior observations that MSCs isolated from different patients grow at different rates, however, it has not been possible to predict this behaviour until now. Since MSCs can only be cultured for a finite length of time after isolation from a patient, and additionally since it is desirable that the length of time it takes to generate large amounts of MSCs (for subsequent uses such as therapeutic uses) is as short as possible, it is important to be able to predict as soon as practically possible after MSC isolation whether it has high proliferation capacity. The present discovery provides the advantage of predicting the high proliferation capacity of MSCs as little as one cell passage after isolation from a patient, using only one gene marker, or its antisense counterpart. One notable advantage of the present discovery is the brevity of time after MSC isolation within which MSC proliferation capacity can be determined. Another notable advantage of the present discovery is the ability to determine proliferation capacity by means other than measuring the proliferation capacity per se directly. It was not known that the specific GAS6 gene and its antisense counterpart GAS6-AS1 would be predictive or determinative of MSC proliferation capacity, nor was it expected by the inventors that such predictions could be made using only these genes.

### Mesenchymal Stem Cell (MSC)

Aspects of the present invention relate to mesenchymal stem cells (MSCs) and populations thereof. An MSC may be generally defined as a stromal cell which is multipotent and has the ability to self-renew (i.e., self-replicate). “Multipotency” refers to a cell's ability to differentiate into multiple, or more than one, different cell types. MSCs may differentiate into various cell types such as chondrocytes, adipocytes, blood cells, etc.

A skilled person will be aware of methods to identify a cell as being an MSCs, including but not limited to, techniques discussed in the Examples hereinbelow.

Suitably, a cell may be identified as being an MSCs by the presence and/or absence of expression of a characteristic set of one or more markers. Suitably the marker(s) may comprise cluster of differentiation (CD) antigens, suitably which are expressed by MSCs. Suitably an MSC may express one or more of the following markers: CD90 and CD105. Suitably an MSC may express CD90. Suitably an MSC may express CD105. In some embodiments, an MSC may express CD90 and CD105, otherwise referred to as CD90+ and CD105+.

Suitably, an MSC may lack expression one or more of the following markers: CD34 and CD45. Suitably, an MSC may lack expression of CD34. Suitably, an MSC may lack expression of CD45. In some embodiments, an MSC may lack expression CD34 and CD45, otherwise referred to as CD34− and CD45−. In some embodiments, an MSC may express CD90 and CD105, otherwise referred to as CD90+ and CD105+, and may lack expression of CD34 and CD45, otherwise known as CD34− and CD45− (i.e., MSCs may be CD90+, CD105+, CD34−, CD45−).

Suitably, a population of cells may be determined as being a population of MSC cells by a high and/or low proportion of cells expressing a characteristic set of one or more markers. Suitably the marker(s) may comprise cluster of differentiation (CD) antigens, suitably which are expressed by MSCs. Suitably a population of MSCs may be one where a high proportion of cells express one or more of the following markers: CD90 and CD105. Suitably a population of MSCs may be one where a high proportion of cells express CD90. Suitably, more than 90% of cells may express CD90. Suitably a population of MSCs may be one where a high proportion of cells express CD105. Suitably, more than 90% of cells may express CD105. Suitably, a population of MSCs may be one where a high proportion of cells express CD90 and CD105. In some embodiments, a population of MSCs may be one where more than 90% of cells express CD90 and CD105.

Suitably a population of MSCs may be one where a low proportion of cells express one or more of the following markers: CD34 and CD45. Suitably a population of MSCs may be one where a low proportion of cells express CD34. Suitably, less than 30% of cells may express CD34. Suitably a population of MSCs may be one where a low proportion of cells express CD45. Suitably, less than 30% of cells may express CD45. Suitably, a population of MSCs may be one where a low proportion of cells express CD34 and CD45. In some embodiments, a population of MSCs may be one where less than 30% of cells express CD34 and CD45.

In some embodiments, a population of MSCs may be one where more than 90% of cells express CD90 and CD105 and less than 30% of cells express CD34 and CD45.

The level of expression of the one or more markers may be ascertained by any suitable technique that is known to a person skilled in the art. Merely by way of example, marker expression levels may be ascertained through measurement of the levels of the corresponding gene transcripts or corresponding protein product. Suitable means to do this may include, for example, by staining cells with antibodies specifically binding the markers of interest and identifying stained cells by cell sorting technologies, or nucleotide amplification methods such as quantitative PCR (qPCR, a.k.a., real-time PCR).

Suitably, MSCs used in the present invention may be derived from a variety of tissue types including, for example, from adipose tissue, bone marrow, umbilical cord tissue, blood, liver, dental pulp, and skin. Suitably, the MSCs may be selected from adipose tissue-derived MSCs (ADSCs), bone marrow-derived MSCs (MB-MSCs), and umbilical cord tissue-derived mesenchymal stem cells (UC-MSCs), or any combination thereof. In one embodiment, the MSCs may be adipose-derived MSCs. In another embodiment, the MSCs may be bone marrow-derived MSCs. Suitably, the MSCs may be derived from humans or other mammals. Alternatively, the MSCs may be derived from existing in vitro-maintained MSC cell lines or populations, for example, from existing MSC cell lines maintained in cell line repositories.

Suitably reference herein to a ‘population’ of MSCs refers to a collection of more than one MSC, suitably a collection of a plurality of MSCs. Suitably a population of MSCs may be derived from the same tissue or from different tissues as listed above.

In the context of aspects of the present invention where MSCs, pharmaceutical compositions comprising populations of MSCs, combinations of MSCs, or formulations of MSCs are used as medicaments, suitably the MSCs may be autologous cells or allogeneic cells. “Autologous cells” refers to cells which are intended for subsequent therapeutic use in the same subject from which the MSC was originally derived. “Allogeneic cells” refers to cells which are intended for subsequent therapeutic use in a different subject from which the MSC was originally derived. Suitably, allogeneic cells may be cells derived from one human subject and intended for subsequent therapeutic use in another human subject.

Suitably, the MSCs used in the present invention may be wild-type cells or may be modified cells, such as genetically engineered cells. Suitably the use of the GAS6 markers described herein can equally apply to wild type or modified MSCs.

### Proliferative Capacity

Aspects of the present invention relate to determining, identifying or selecting MSCs having a high proliferative capacity, MSCs having a high proliferative capacity, populations thereof, and their subsequent uses.

As used herein, an MSC with high proliferative capacity is one that proliferates, or that is capable of proliferating, at a faster rate, and/or one which proliferates, or is capable of proliferating, for a longer duration of time prior to senescence, suitably when compared to the average proliferation rate and/or average proliferation duration in a population of MSCs. Suitably in a typical population of MSCs. Suitably in a heterogenous population of MSCs. Suitably a typical population of MSCs may be a heterogenous population of MSCs. “Heterogeneous population of MSCs” or “heterogeneous population of cells” refers to a population of MSCs wherein constituent MSCs have been derived from a sufficiently diverse range of source samples to capture a reasonably large degree of diversity in proliferation capacity. Suitable heterogenous populations of MSCs are available to order, or may be prepared by mixing several different source samples together to form a heterogenous population of cells. Suitably an MSC with high proliferative capacity may be one that proliferates, or that is capable of proliferating, at a faster rate, and/or one which proliferates, or is capable of proliferating, for a longer duration of time prior to senescence, when compared to the average proliferation rate and/or average proliferation duration in the population of MSCs from which the cell is to be identified or selected.

Suitably an MSC with high proliferative capacity is one that has, or is capable of having, a high growth rate and/or a long growth phase, suitably when compared to the average growth rate and/or average growth phase in a population of MSCs, suitably which may be a heterogenous population of MSCs. Suitably an MSC with high proliferative capacity is one that has, or is capable of having, a high growth rate and/or a long growth phase, when compared to the average growth rate and/or average growth phase in the population of MSCs from which the cell is to be identified or selected.

Suitably therefore ‘high proliferative capacity’ may equally refer to the capability of an MSC in the future, or may refer to its current status. Suitably therefore the MSC may be capable of having a high growth rate and/or a long growth phase in the future, or may already have a high growth rate and/or a long growth phase.

“Senescence” refers to cellular growth arrest which may be accompanied by cellular decay or apoptosis. “Apoptosis” refers to programmed cell death.

In one embodiment, an MSC with high proliferative capacity is one that is capable of growing faster, or one which grows faster than another MSC, or faster than the average of a population of MSCs, suitably which may be a heterogenous population of MSCs. “Faster growth” refers herein to the growth rate, or average growth rate, of an MSC with high proliferative capacity. Suitably therefore the MSC may have a high growth rate, or may be capable of having a high growth rate. Suitably therefore the MSC may have a higher growth rate, or may be capable of having a higher growth rate, than another MSC or than the average growth rate of a population of MSCs, suitably which may be a heterogenous population of MSCs. Suitably, a faster growth rate or a higher growth rate may be established in comparison to the average growth rate in the same population of cells as the MSC being established as having faster growth or higher growth rate, or in comparison to the average growth rate of another different population of cells, suitably which may be a heterogenous population of MSCs.

MSC growth rates may be determined by any suitable means known to those in the art, including the techniques described in the Examples.

Suitably, growth rates may be measured by determining cell doubling times (i.e., population doubling times, when the cell is grown into a population). “Cell doubling time” refers to the average time it takes for a cell to divide into two cells. Suitably, population doubling times (PDTs) can be calculated using the formula: PDT=t×log(2)/log(cells harvested/cells seeded), where t=the time between cell seeding and cell harvesting.

Suitably, the cell doubling time (or PDT when grown into a population) of an MSC with high proliferative capacity may be below 20 days. Suitably below 19, 18, 17, 16, 15, 14, 13, 12, 11, 10, 9, 8, 7, 6, or 5 days. In one embodiment, below 15 days. In one embodiment, below 10 days. In one embodiment, below 5 days.

Suitably an MSC having a high growth rate may be defined as having a cell doubling time (or PDT when grown into a population) of below 20 days. Suitably below 19, 18, 17, 16, 15, 14, 13, 12, 11, 10, 9, 8, 7, 6, or 5 days. In one embodiment, below 15 days. In one embodiment, below 10 days. In one embodiment, below 5 days. Suitably the cell doubling time may be an average cell doubling time or PDT that is measured over a range of passage numbers.

Alternatively, or additionally growth rates may be determined by measuring appropriate cellular markers indicative of cellular growth such as the expression levels of certain genes, levels of certain RNA transcripts, and/or levels of certain proteins.

Alternatively or additionally, growth rates may be determined by quantifying cellular DNA synthesis for example by staining techniques involving BrdU or other chemical reagents commonly used for this purpose.

Proliferation at a faster rate or high growth rate may occur at any stage in the lifetime of an MSC cell line. “Lifetime” refers to the span of time during which an MSC cell line is actively growing, suitably before senescence.

Suitably, proliferation at a faster rate or a high growth rate may occur, or may be determined, at cell passage number 1 (P1), P2, P3, P4, P5, P8, P10, P15, P20, P25, P30 and/or higher passage numbers. “Passage number” or “number of passages” refers to the number of times a cell line has been re-seeded to initiate a new cell culture. Suitably, proliferation at a faster rate or a high growth rate may occur, or may be determined, at higher passage numbers, for example P10, P11, P12, P13, P14, P15, P20, P25, P30 and/or higher. Suitably, proliferation at a faster rate or a high growth rate may occur or may be determined, during P1-P15. Used herein, “-” refers to a range. Suitably, proliferation at a faster rate or a high growth rate may occur during P1-P5, P6-P10, and/or P11-P15. In one embodiment, proliferation at a faster rate or a high growth rate occurs at or by P10. In another embodiment, proliferation at a faster rate or a high growth rate occurs at or by P15.

Suitably therefore, an MSC with high proliferative capacity may comprise a low cell doubling time (or PDT when grown into a population) of below 20 days, below 15 days, below 10 days or below 5 days, at cell passage number 1 (P1), P2, P3, P4, P5, P8, P10, P15, P20, P25, P30 and/or higher passage numbers; at higher passage numbers, for example P10, P11, P12, P13, P14, P15, P20, P25, P30 and/or higher; during P1-P15; during P1-P5, P6-P10, and/or P11-P15; at P10; or at P15.

Suitably therefore, an MSC with a high growth rate may comprise a low cell doubling time (or PDT when grown into a population) of below 20 days, below 15 days, below 10 days or below 5 days, at cell passage number 1 (P1), P2, P3, P4, P5, P8, P10, P15, P20, P25, P30 and/or higher passage numbers; at higher passage numbers, for example P10, P11, P12, P13, P14, P15, P20, P25, P30 and/or higher; from P1-P15; from P1-P5, P6-P10, and/or P11-P15; at P10; or at P15.

Suitably for high proliferative capacity MSCs, cell or population doubling time is maintained below 20 days, below 15 days, below 10 days or below 5 days for high number of passages. Suitably for high proliferative capacity MSCs, cell or population doubling time is maintained below 20 days, below 15 days, below 10 days or below 5 days from at least P1 to P10, suitably from P1 to P15. In one embodiment, an MSC with high proliferative capacity, or a high growth rate, retains a cell doubling time (or PDT when grown into a population) of below 10 days from passage number P1 to P15.

Suitably therefore, for completeness, an MSC with a low proliferative capacity is essentially the opposite to the definitions provided above. Suitably an MSC with low proliferative capacity is one that proliferates, or that is capable of proliferating, at a slower rate, suitably when compared to the average proliferation rate and/or average proliferation duration in a population of MSCs, suitably which may be a heterogenous population of MSCs. Suitably when compared to the average proliferation rate and/or average proliferation duration in the population of MSCs from which the cell is to be identified or selected. Suitably an MSC with low proliferative capacity is one that has, or is capable of having, a low growth rate, suitably when compared to the average growth rate and/or average growth phase in a population of MSCs, suitably which may be a heterogenous population of MSCs. Suitably when compared to the average growth rate and/or average growth phase in the population of MSCs from which the cell is to be identified or selected.

Suitably an MSC with a low proliferative capacity, or low growth rate, may comprise a high cell doubling time (or PDT when grown into a population) of above 10 days, above 15 days, above 20 days at cell passage number 1 (P1), P2, P3, P4, P5, P8, P10, P15, P20, P25, P30 and/or higher passage numbers; at higher passage numbers, for example P10, P11, P12, P13, P14, P15, P20, P25, P30 and/or higher; during P1-P15; during P1-P5, P6-P10, and/or P11-P15; at P10; or at P15.

Suitably, the doubling time increases with passage number for low proliferative capacity, or low growth rate MSCs. Suitably therefore an MSC with low proliferative capacity, or low growth rate may comprise a high cell doubling time (or PDT when grown into a population) of above 10 days, above 15 days, above 20 days at higher passage numbers, for example P10, P11, P12, P13, P14, P15, P20, P25, P30 and/or higher. Suitably a high cell doubling time (or PDT when grown into a population) of above 10 days at P10 or higher. Suitably a high cell doubling time (or PDT when grown into a population) of above 15 days at P15 and higher.

In one embodiment, an MSC with a high proliferative capacity is one capable of growing for a longer duration of time prior to reaching senescence than another MSC, or longer than the average duration of time prior to reaching senescence of a population of MSCs, suitably which may be a heterogenous population of MSCs. Suitably, an MSC with high proliferative capacity may therefore be one which grows for a longer duration of time prior to senescence than another MSC, or longer than the average duration of time prior to reaching senescence of a population of MSCs, suitably which may be a heterogenous population of MSCs.

“Longer duration of growth” refers herein to the duration of growth i.e. the growth phase, in other words, the time for which a cell is capable of growing prior to senescence. For an MSC with high proliferative capacity, suitably a longer duration of growth may refer to a longer growth phase. Suitably therefore the MSC may have a long growth phase, or may be capable of having a long growth phase. Suitably therefore the MSC may have a longer growth phase, or may be capable of having a longer growth phase than average growth phase of a population of MSCs, suitably which may be a heterogenous population of MSCs.

“Duration of growth” or “growth phase” further excludes periods of time during which growth of the cell is arrested, for example through freezing at −20° C. or −80° C. Suitably, a longer duration of growth or a longer growth phase may be established in comparison to the average duration of growth or growth phase of the same population of cells as the MSC, or in comparison to the average duration of growth or growth phase of another different population of cells, suitably which may be a heterogenous population of MSCs.

Duration of growth or the growth phase of an MSC may be determined by any suitable means known to those in the art, including the techniques described in the Examples.

Suitably, duration of growth or the growth phase of an MSC may be measured by the number of passages the cell line can grow for before senescence. Suitably, MSCs with high proliferative capacity may be capable of growing, or may grow, for a duration of more than eighteen passages (P18), P19, P21, P22, P23, P24, P25, P26, P27, P28, P29, P30 and/or more passages. In one embodiment, MSCs with high proliferative capacity may be capable of growing, or may grow, for a duration of P18 or more passages. In another embodiment, MSCs with high proliferative capacity may be capable of growing, or may grow, for a duration of P20 or more passages. In another embodiment, MSCs with high proliferative capacity may be capable of growing, or may grow, for a duration of P22 or more passages. In another embodiment, MSCs with high proliferative capacity may be capable of growing, or may grow, for a duration of 24 or more passages.

Suitably therefore an MSC having a long growth phase may be defined as having a growth phase of more than eighteen passages (P18), P19, P21, P22, P23, P24, P25, P26, P27, P28, P29, P30 and/or more passages. In one embodiment, an MSC having a long growth phase may be defined as having a growth phase of P18 or more passages. In one embodiment, an MSC having a long growth phase may be defined as having a growth phase of P20 or more passages. In one embodiment, an MSC having a long growth phase may be defined as having a growth phase of P22 or more passages. In one embodiment, an MSC having a long growth phase may be defined as having a growth phase of P22 or more passages. In one embodiment, an MSC having a long growth phase may be defined as having a growth phase of P24 or more passages.

Alternatively or additionally, duration of growth or the growth phase of an MSC may be measured by the number of generations of cell divisions, or average number of generations of cell divisions, the cell has undergone since the cell line was initialized from a tissue sample. The number of generations of cell divisions is also referred to as the number of population doublings, or, in the case of a population of cells, is also referred to as the cumulative population doublings. Determination of the number of population doublings is described in the Examples. Suitably, population doublings (PDs) can be calculated using the following formula: PDs=[log(Number of Harvested MSCs)−log(Number of seeded MSCs)]/log(2)].

Suitably, MSCs with high proliferative capacity may be capable of growing, or may grow, for a duration of 44, 45, 46, 47, 48, 49, 50, 51, 52, 53, 54, 55, 56, 57, 58, 59, 60 or more generations of cell divisions, or population doublings. In one embodiment, MSCs with high proliferative capacity may be capable of growing, or may grow, for a duration of 44 or more generations, or population doublings. In another embodiment, MSCs with high proliferative capacity may be capable of growing, or may grow, for a duration of 50 or more generations, or population doublings. In another embodiment, MSCs with high proliferative capacity may be capable of growing, or may grow, for a duration of 55 or more generations, or population doublings. In another embodiment, MSCs with high proliferative capacity may be capable of growing, or may grow, for a duration of 60 or more generations, or population doublings.

Suitably therefore an MSC having a long growth phase may be defined as having a growth phase of 44, 45, 46, 47, 48, 49, 50, 51, 52, 53, 54, 55, 56, 57, 58, 59, 60 or more generations of cell divisions, or population doublings. In one embodiment, an MSC having a long growth phase may be defined as having a growth phase of 44 or more generations, or population doublings. In one embodiment, an MSC having a long growth phase may be defined as having a growth phase of 50 or more generations, or population doublings. In one embodiment, an MSC having a long growth phase may be defined as having a growth phase of 55 or more generations, or population doublings. In one embodiment, an MSC having a long growth phase may be defined as having a growth phase of 60 or more generations, or population doublings.

Suitably therefore, for completeness, an MSC with a low proliferative capacity is essentially the opposite to the definitions provided above. Suitably an MSC with low proliferative capacity is one that proliferates, or that is capable of proliferating, for a shorter duration of time prior to senescence, suitably when compared to the average proliferation rate and/or average proliferation duration in a population of MSCs, suitably which may be a heterogenous population of MSCs. Suitably when compared to the average proliferation rate and/or average proliferation duration in the population of MSCs from which the cell is to be identified or selected. Suitably an MSC with low proliferative capacity is one that has, or is capable of having, a short growth phase, suitably when compared to the average growth rate and/or average growth phase in a population of MSCs, suitably which may be a heterogenous population of MSCs. Suitably when compared to the average growth rate and/or average growth phase in the population of MSCs from which the cell is to be identified or selected.

Suitably therefore an MSC having a short growth phase may be defined as having a growth phase of less than eighteen passages (P18), suitably less than P17, P16, P15, P14, P13, P12, P11, P10, P9, P8, P7, P6, P5, P4, P3, P2, or P1 passages. In one embodiment, an MSC having a short growth phase may be defined as having a growth phase of P18 or fewer passages.

Suitably therefore an MSC having a short growth phase may be defined as having a growth phase of less than 60 generations of cell divisions, suitably less than 59, 58, 57, 56, 55, 54, 53, 52, 51, 50, 49, 48, 47, 46, 45, or 44 generations of cell divisions. In one embodiment, an MSC having a short growth phase may be defined as having a growth phase of 44 or fewer generations.

Suitably any of the growth measurements defining proliferative capacity provided hereinabove may be measured when culturing the MSC. Suitably when culturing the MSC in typical media and under typical culture conditions. Exemplary conditions for culturing the MSC are provided in the examples herein, but will also be known in the art, such as from Salerno et al Stem Cells 2020, 38:1438-53 and Kafienah et al Stem Cells 2006, 24:1113-20. For example, any of the growth measurements recited herein may be measured when culturing an MSC under the following conditions: in medium comprising low glucose Dulbecco's Modified Eagles Medium (Sigma) supplemented with 10% (v/v) Foetal Bovine Serum (FBS, Thermo Scientific Hyclone, Loughborough, UK), 1% (v/v) Glutamax (Sigma) and 1% (v/v) Penicillin/Streptomycin (Sigma). Optionally the medium may be supplemented with 10 ng/ml FGF-2 (Peprotech). Suitably the cell suspension may then be isolated, for example by repeated washing with media. Suitably the cells may then be pelleted and resuspended, for example by centrifuging at 500 g for 5 minutes and the supernatant/fat removed. The resulting cell pellet may then be resuspended in medium, and plated at a seeding density of for example between 1.5-2.0×10⁵ nucleated cells per cm². Suitably the cells may then be incubated in the same medium, for example at 37° C. in a humidified atmosphere of 5% CO₂ and 95% air. Suitably the medium is changed regularly, for example every four days to every other day, suitably until adherent cells reached 90% confluence. Suitably the cells may then be passaged.

### Gene Expression of GAS6 and GAS6-AS1

Aspects of the present invention relate to obtaining and using data indicative of the gene expression levels of GAS6, GAS6-AS1, and/or the ratio of GAS6-AS1:GAS6 gene expression levels.

“Gene expression” refers to the transcription of a gene into an RNA product. “Gene expression levels” therefore refer to the abundance of RNA transcript being produced from a gene, irrespective of the lifetime of the transcribed RNA, which, for example, may be swiftly degraded and thus have short lifetimes.

Data indicative of gene expression and/or gene expression levels may refer to data containing information regarding RNA transcript presence, relative or absolute RNA transcript abundance, and/or the ability of genes to produce RNA transcripts such as DNA chromatin signatures. Suitably, data indicative of gene expression and/or gene expression levels may be, for example, DNA or RNA sequencing data, or fluorescent imaging data.

Suitably, gene expression data (i.e., data indicative of gene expression, i.e., data indicative of gene expression levels) may be obtained by, for example, quantitative polymerase chain reaction (qPCR, a.k.a., real time PCR), high throughput transcriptomic sequencing (a.k.a., RNA sequencing or RNA-seq), in vivo fluorescent nucleotide imaging, DNA/RNA microarray analysis, expressed sequence tag (EST) sequencing, Sanger sequencing of complementary DNAs (cDNAs), digital PCR (a.k.a., droplet digital PCR), and Northern blotting, amongst any other method that may be known in the art.

Suitably, gene expression or gene expression levels may be measured in suitable units known in the art, for example gene counts, transcript counts, read counts, trimmed mean of m-value (TMM), or delta Ct (i.e., ΔCt) values. As is understood by the skilled person, the specific values measured and the units of measurement will often be determined by the technique chosen to measure the gene expression which may be any of those described herein.

Alternatively or additionally, data indicative of gene expression levels may refer to data containing information regarding protein expression, suitably protein expression from the gene indicated. Suitably the expression of GAS6 protein. Suitable protein expression may refer to information regarding protein presence, relative or absolute protein abundance, or protein activity. Suitably, data indicative of protein expression may be, for example, western blot data or fluorescent imaging data.

Suitably, protein expression data may be obtained by, for example, western blot, fluorescent imaging, semi-quantitative SDS-PAGE software tools such as ImageJ, mass spectrometry, and ELISA.

In one embodiment, obtaining data indicative of the levels of gene expression of one or more of GAS6 and GAS-AS1 comprises measuring the gene expression levels of GAS6 and GAS-AS1. Suitably by any of the techniques listed above. In one embodiment, by RNA-seq or by qPCR.

Examples of ways of obtaining gene expression data may be found in the Examples.

GAS6 (growth arrest-specific gene 6) is a gene expressed in humans involved in the stimulation of cell proliferation. GAS6-AS1 (GAS6 antisense RNA 1) is a non-protein-coding RNA which is the antisense sequence of GAS6 involved in the regulation of GAS6.

Suitably the GAS6 gene may comprise a nucleic acid sequence according to SEQ ID NO: 1 or a sequence with at least 70%, 75%, 80%, 85%, 90%, 91%, 92%, 93%, 94%, 95%, 96%, 97%, 98%, 99% identity to SEQ ID NO: 1. Suitably, the GAS6 gene may comprise the GAS6 coding sequence (CDS) according to SEQ ID NO: 2 or a sequence with at least 70%, 75%, 80%, 85%, 90%, 91%, 92%, 93%, 94%, 95%, 96%, 97%, 98%, 99% identity to SEQ ID NO: 2. A translation of SEQ ID NO: 2 is provided in SEQ ID NO: 3. Suitably, GAS6 may be any gene in a public database which is annotated as GAS6 and is believed to fulfil the same function as the GAS6 found in humans.

Suitably the GAS6-AS1 gene may comprise a nucleic acid sequence according to SEQ ID NO: 4 or a sequence with at least 70%, 75%, 80%, 85%, 90%, 91%, 92%, 93%, 94%, 95%, 96%, 97%, 98%, 99% identity to SEQ ID NO: 4. Suitably, GAS6-AS1 may be any gene in a public database which is annotated as GAS6-AS1 and is believed to fulfil the same function as the GAS6-AS1 found in humans.

The ratio of GAS6-AS1:GAS6 gene expression levels may be calculated by dividing the gene expression level of GAS6-AS1 by the gene expression level of GAS6. Suitably the expression levels comprise a numerical data value indicative of the expression level. Numerical data values indicative of the gene expression of GAS6 and/or GAS6-AS1 may be obtained as described above by, for example, quantitative polymerase chain reaction (qPCR, a.k.a., real time PCR), high throughput transcriptomic sequencing (a.k.a., RNA sequencing or RNA-seq), in vivo fluorescent nucleotide imaging, DNA/RNA microarray analysis, expressed sequence tag (EST) sequencing, Sanger sequencing of complementary DNAs (cDNAs), digital PCR (a.k.a., droplet digital PCR), and Northern blotting, amongst any other method that may be known in the art. Suitably the ratio is a relative measurement of the level of expression of the GAS6-AS1 gene compared to the level of expression of the GAS6 gene. Suitably therefore any numerical data value indicative of gene expression level of GAS6 and GAS6-AS1 may be used to calculate the ratio thereof, as long as the same type of numerical data is used for each gene.

Suitably in any of the aspects of the invention, any combination of the level of expression of the GAS6 gene, the level of expression of the GAS6-AS1 gene and/or the ratio of gene expression of GAS6-AS1:GAS6 may be used to indicate a mesenchymal stem cell having a high proliferative capacity. Suitably the level of expression of one or more of these genes, or the ratio thereof, may be used as a marker of high proliferative capacity.

In one embodiment of any of the aspects of the invention, the marker used to indicate high proliferative capacity of an MSC is the level of expression of the GAS6 gene. Suitably in such an embodiment, the methods of the invention may comprise the following steps:
a. Obtaining data indicative of the level of expression of the GAS6 gene in the mesenchymal stem cell;
b. Comparing the level of expression of step (a), to a reference value;
c. Determining, identifying or selecting the mesenchymal stem cell having a high proliferative capacity if level of expression of the GAS6 gene, is below the reference value.

In one preferred embodiment of any of the aspects of the invention, the marker used to indicate high proliferative capacity of an MSC is the level of expression of the GAS6-AS1 gene. Suitably in such an embodiment, the methods of the invention may comprise the following steps:
a. Obtaining data indicative of the level of expression of the GAS6-AS1 gene in the mesenchymal stem cell;
b. Comparing the level of expression of step (a), to a reference value;
c. Determining, identifying or selecting the mesenchymal stem cell having a high proliferative capacity if level of expression of the GAS6-AS1 gene, is below the reference value.

In one more preferred embodiment of any of the aspects of the invention, the marker used to indicate high proliferative capacity of an MSC is the ratio of gene expression of GAS6-AS1:GAS6. Suitably in such an embodiment, the methods of the invention may comprise the following steps:
a. Obtaining data indicative of the level of expression of one or both of the following genes: GAS6 and GAS6-AS1 in the mesenchymal stem cell;
b. Calculating a ratio of the level of gene expression of GAS6-AS1:GAS6;
c. Comparing the ratio of step (b), to a reference value;
d. Determining, identifying or selecting the mesenchymal stem cell having a high proliferative capacity if the ratio of GAS6-AS1:GAS6 gene expression, is below the reference value.

### Reference Value

Aspects of the present invention relate to comparing the gene expression levels of GAS6, GAS6-AS1, and/or the ratio of GAS6-AS1:GAS6 gene expression levels, to a reference value, in order to determine, identify or select an MSC having high proliferative capacity.

Suitably, as will be understood by a person skilled in the art, the reference value may be regarded as a typical value in a population of MSCs, suitably the typical gene expression level of GAS6, GAS6-AS1, and/or the ratio of GAS6-AS1:GAS6 gene expression in a population of MSCs. Suitably, the reference value may be regarded as an average value thereof in a population of MSCs, suitably the average gene expression level of GAS6, GAS6-AS1, and/or the ratio of GAS6-AS1:GAS6 gene expression in a population of MSCs. Suitably this population of MSCs may be regarded as a reference population.

Suitably it will be understood that the reference value will depend on the measurement technique being used, and will depend on the desired proliferative capacity that is being selected for. Suitable measurement techniques for measuring gene expression levels are described herein. In some embodiments, the measurement technique is RNA-seq.

Suitably therefore the reference value may be the median level of expression of the GAS6 gene, the GAS6-AS1 gene, and/or the median ratio of GAS6-AS1:GAS6 gene expression in a reference population of MSCs.

Suitably therefore the reference value may be the mean level of expression of the GAS6 gene, the GAS6-AS1 gene, and/or the median ratio of GAS6-AS1:GAS6 gene expression in a reference population of MSCs.

Suitably a reference population may be a typical population of MSCs. Suitably the reference population may be a heterogenous population of MSCs as defined hereinabove. Suitable heterogenous populations are available in the art or may easily be prepared and measured. In some embodiments, the reference population may be a population of MSCs having a similar number of low proliferative capacity MSCs and high proliferative capacity MSCs, suitably having about equal numbers of low proliferative capacity MSCs and high proliferative capacity MSCs, wherein low and high proliferative capacity MSCs are as defined elsewhere herein. Suitably such a reference population may be prepared from available MSC populations or samples that are known to have a high or low proliferation capacity, such populations are known in the art and available to order. Alternatively various MSC samples from different sources or samples can be collected and cultured for a suitable length of time to determine their proliferation capacity, by traditionally measuring growth rate or other parameters as defined above, to find a suitable high and low proliferative capacity cells which may then be combined to form a reference population.

Suitably the reference value may in some cases be the mean level of expression of the GAS6 gene, the GAS6-AS1 gene, and/or the median ratio of GAS6-AS1:GAS6 gene expression in the population of MSCs from which the MSC of interest is derived.

Suitably the reference value may in some cases be the median level of expression of the GAS6 gene, the GAS6-AS1 gene, and/or the median ratio of GAS6-AS1:GAS6 gene expression in the population of MSCs from which the MSC of interest is derived.

Suitably the median level of gene expression is determined by obtaining data indicative of the level of expression of said gene or genes in each MSC of a population of MSCs, suitably using standard expression measurement techniques as described herein, then arranging the data points of the level of expression in each MSC from smallest to largest, and selecting the level of expression which is the middle data point in the list. Suitably the median level of gene expression is determined in the same way, using the same technique/method, as for the MSC of interest.

Suitably the reference value may be the mean or median relative expression level of the GAS6 gene, the GAS6-AS1 gene, and/or the median ratio of GAS6-AS1:GAS6 relative gene expression in a reference population of MSCs. Suitably wherein the reference population is a population of MSCs having a similar number of low proliferative capacity MSCs and high proliferative capacity MSCs, suitably having about equal numbers of low proliferative capacity MSCs and high proliferative capacity MSCs, wherein low and high proliferative capacity MSCs are as described elsewhere herein.

Suitably the reference value may be the mean relative expression level of the GAS6 gene, the GAS6-AS1 gene, and/or the median ratio of GAS6-AS1:GAS6 relative gene expression in the population of MSCs from which the MSC of interest is derived.

Suitably the reference value may be the median relative expression level of the GAS6 gene, the GAS6-AS1 gene, and/or the median ratio of GAS6-AS1:GAS6 relative gene expression in the population of MSCs from which the MSC of interest is derived.

“Relative expression” as used herein refers to the expression level of the gene of interest relative to a normalisation factor.

Suitably, a normalisation factor may be the size of the gene expression data source, suitably the library size, suitably when using high throughput transcriptomic sequencing (a.k.a., RNA sequencing or RNA-seq) for example. Suitably, a normalisation factor may be the number of mapped reads. “Mapped reads” are define herein as the number of reads or fragments from a library which have been mapped onto an appropriate reference genome, suitably from a high throughput transcriptomic sequencing (a.k.a., RNA sequencing or RNA-seq) library for example, suitably which may be measured in counts per million (CPM), or reads per million mapped reads (RPM). Suitably, a normalisation factor may be the size of gene expression data source, suitably library size as mentioned above, optionally in addition to the length of the target transcript which corresponds to the gene being measured, suitably which may be measured in Reads per kilo base of transcript per million mapped reads (RPKM). Suitably, a normalisation factor may be the expression level of a control gene used for normalising results across samples, suitably which may be measured in any units. A “control gene” may be any gene commonly used for normalisation purposes in the art, suitably any gene that is ubiquitously expressed at stable levels in different biological contexts, such as a housekeeping gene. Suitable control genes may be: GAPD, ACTB, B2M, TUBA, G6PD, LDHA, and HPRT.

The reference value for the level of GAS6 gene expression is suitably above the value of GAS6 gene expression found in MSCs which have been determined as having high proliferative capacity, wherein high proliferative capacity has been established as described elsewhere herein. Suitably establishing whether a population has high proliferative capacity can be done by culturing said population and measuring one or more of the growth characteristics defined above.

In the same way, the reference value for the level of GAS6 gene expression is suitably the same as or below the value of GAS6 gene expression found in MSCs which have been determined as having low proliferative capacity, wherein low proliferative capacity has been established as described elsewhere herein. Suitably establishing whether a population has low proliferative capacity can be done by culturing said population and measuring one or more of the growth characteristics defined above.

Suitably, therefore the reference value for the level of GAS6 gene expression is higher than the level of GAS6 gene expression of those MSCs which have been determined as having high proliferative capacity, wherein high proliferative capacity has been established as described elsewhere herein. Suitably establishing whether a population has high proliferative capacity can be done by culturing said population and measuring one or more of the growth characteristics defined above.

Suitably, the reference value for the level of GAS6 gene expression may be above the value of relative expression of GAS6 found in MSCs which have been determined as having high proliferative capacity, wherein high proliferative capacity has been established as described elsewhere herein, and wherein the relative expression of GAS6 is the expression level of GAS6 relative to the expression level of a control gene. Suitably establishing whether a population has high proliferative capacity can be done by culturing said population and measuring one or more of the growth characteristics defined above.

Suitably, therefore the reference value for the level of GAS6 gene expression is higher than the relative expression of GAS6 of those MSCs which have been determined as having high proliferative capacity, wherein high proliferative capacity has been established as described elsewhere herein, and wherein the relative expression of GAS6 is the expression level of GAS6 relative to the expression level of a control gene. Suitably establishing whether a population has high proliferative capacity can be done by culturing said population and measuring one or more of the growth characteristics defined above.

In one embodiment, the MSC has a high proliferative capacity if its level of GAS6 gene expression is the same as or below the level of GAS6 gene expression found in MSCs which have been determined as having low proliferative capacity, wherein low proliferative capacity has been established as described elsewhere herein. Suitably establishing whether a population has low proliferative capacity can be done by culturing said population and measuring one or more of the growth characteristics defined above.

A reference value for GAS6 gene expression may be determined at cell passage number 1 (P1), P2, P3, P4, P5, P8, P10, P15, P20, P25, P30 and/or higher passage numbers. Suitably, the reference value for GAS6 gene expression may be determined at P1. Suitably, the reference value for GAS6 gene expression may be determined during P1-P10. In one embodiment, the reference value for GAS6 gene expression may be determined at P1. In one embodiment, the reference value for GAS6 gene expression may be determined at P2. In one embodiment, the reference value for GAS6 gene expression may be determined at P3. In one embodiment, the reference value for GAS6 gene expression may be determined at P4. In one embodiment, the reference value for GAS6 gene expression may be determined at P5. In one embodiment, the reference value for GAS6 gene expression may be determined at P6. In one embodiment, the reference value for GAS6 gene expression may be determined at P7. In one embodiment, the reference value for GAS6 gene expression may be determined at P8. In one embodiment, the reference value for GAS6 gene expression may be determined at P9. In one embodiment, the reference value for GAS6 gene expression may be determined at P10.

A reference value for GAS6 gene expression may be determined by the same techniques/method by which the level of GAS6 gene expression may be determined. Techniques for determining gene expression levels are described elsewhere in this description. In one embodiment, GAS6 gene expression levels may be determined by high throughput transcriptomic sequencing (a.k.a., RNA sequencing or RNA-seq), optionally followed by processing using various software packages as described in the Examples. In one embodiment, GAS6 gene expression levels may be determined by quantitative polymerase chain reaction (qPCR, a.k.a., real time PCR). In one embodiment, GAS6 gene expression levels may be determined by digital PCR (a.k.a., droplet digital PCR).

Suitably, the reference value for GAS6 gene expression may be measured in units of gene counts, suitably relative gene counts. Suitably, wherein the gene counts are relative (i.e. normalised) to library size, suitably relative to a high throughput transcriptomic sequencing (a.k.a., RNA sequencing or RNA-seq) library size. Suitably the reference value for GAS6 gene expression may be measured in units of counts per million mapped reads (CPM) or reads per million mapped reads (RPM). Suitably gene counts are typically measured in counts per million mapped reads (CPM) or reads per million mapped reads (RPM).

Suitably the reference value for GAS6 gene expression may therefore be 35, 36, 37, 38, 39, 40, 41, 42, 43, 44, 45, 46, 47, 48, 49, 50, 51, 52, 53, 54, 55, 56, 57, 58, 59, or 60 relative gene counts, suitably CPM. Suitably at any passage such as (P1), P2, P3, P4, P5, P8, P10, P15, P20, P25, P30 and/or higher passage numbers, in one embodiment at passage 1 (P1). Suitably, the reference value for GAS6 gene expression may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 relative gene counts, suitably CPM. Suitably at any passage such as (P1), P2, P3, P4, P5, P8, P10, P15, P20, P25, P30 and/or higher passage numbers, in one embodiment at passage P10. Suitably, the reference value for GAS6 gene expression may be 42, 43, or 44 relative gene counts, suitably CPM. Suitably at any passage such as (P1), P2, P3, P4, P5, P8, P10, P15, P20, P25, P30 and/or higher passage numbers, in one embodiment at passage P15.

Suitably therefore, the MSC has a high proliferative capacity if its level of expression of the GAS6 gene is below 35, 36, 37, 38, 39, 40, 41, 42, 43, 44, 45, 46, 47, 48, 49, 50, 51, 52, 53, 54, 55, 56, 57, 58, 59, or 60 relative gene counts, suitably CPM. Suitably at any passage, such as (P1), P2, P3, P4, P5, P8, P10, P15, P20, P25, P30 and/or higher passage numbers, in one embodiment at passage 1 (P1). Suitably therefore, the MSC has a high proliferative capacity if its level of expression of the GAS6 gene is below 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 relative gene counts, suitably CPM. Suitably any passage, such as (P1), P2, P3, P4, P5, P8, P10, P15, P20, P25, P30 and/or higher passage numbers, in one embodiment at P10. Suitably therefore, the MSC has a high proliferative capacity if its level of expression of the GAS6 gene is below 42, 43, or 44 relative gene counts, suitably CPM. Suitably any passage, such as (P1), P2, P3, P4, P5, P8, P10, P15, P20, P25, P30 and/or higher passage numbers, in one embodiment at P15.

Suitably the above reference values may be applicable when measuring the level of GAS6 gene expression using RNA-seq. Suitably therefore the above reference values are as measured using RNA-seq. Optionally one or more further processing steps may be applied to the gene expression data obtained from RNA-seq, such as genome alignment using Bowtie2 and an appropriate reference genome, sequence counts using htseq-count and R library DESeq2 rlog transformation. An exemplary method of measurement of GAS6 gene expression is shown in the examples herein.

The reference value for the level of GAS6-AS1 gene expression is suitably above the value of GAS6-AS1 gene expression found in those MSCs which have been determined as having high proliferative capacity, wherein high proliferative capacity has been established as described elsewhere herein. Suitably establishing whether a population has high proliferative capacity can be done by culturing said population and measuring one or more of the growth characteristics defined above.

In the same way, the reference value for the level of GAS6-AS1 gene expression is suitably the same as or below the value of GAS6-AS1 gene expression found in MSCs which have been determined as having low proliferative capacity, wherein low proliferative capacity has been established as described elsewhere herein. Suitably establishing whether a population has low proliferative capacity can be done by culturing said population and measuring one or more of the growth characteristics defined above.

Suitably, therefore the reference value for the level of GAS6-AS1 gene expression is higher than the level of GAS6-AS1 gene expression of those MSCs which have been determined as having high proliferative capacity, wherein high proliferative capacity has been established as described elsewhere herein. Suitably establishing whether a population has high proliferative capacity can be done by culturing said population and measuring one or more of the growth characteristics defined above.

Suitably, the reference value for the level of GAS6-AS1 gene expression may be above the value of relative expression of GAS6-AS1 found in MSCs which have been determined as having high proliferative capacity, wherein high proliferative capacity has been established as described elsewhere herein, and wherein the relative expression of GAS6-AS1 is the expression level of GAS6-AS1 relative to the expression level of a control gene. Suitably establishing whether a population has high proliferative capacity can be done by culturing said population and measuring one or more of the growth characteristics defined above.

Suitably, therefore the reference value for the level of GAS6-AS1 gene expression is higher than the relative expression of GAS6-AS1 of those MSCs which have been determined as having high proliferative capacity, wherein high proliferative capacity has been established as described elsewhere herein, and wherein the relative expression of GAS6-AS1 is the expression level of GAS6-AS1 relative to the expression level of a control gene. Suitably establishing whether a population has high proliferative capacity can be done by culturing said population and measuring one or more of the growth characteristics defined above.

In one embodiment, the MSC has a high proliferative capacity if its level of GAS6-AS1 gene expression is the same as or below the level of GAS6-AS1 gene expression found in MSCs which have been determined as having low proliferative capacity, wherein low proliferative capacity has been established as described elsewhere herein. Suitably establishing whether a population has low proliferative capacity can be done by culturing said population and measuring one or more of the growth characteristics defined above.

A reference value for GAS6-AS1 gene expression may be determined at cell passage number 1 (P1), P2, P3, P4, P5, P8, P10, P15, P20, P25, P30 and/or higher passage numbers. Suitably, the reference value for GAS6-AS1 gene expression may be determined at P1. Suitably, the reference value for GAS6-AS1 gene expression may be determined during P1-P10. In one embodiment, the reference value for GAS6-AS1 gene expression may be determined at P1. In one embodiment, the reference value for GAS6-AS1 gene expression may be determined at P2. In one embodiment, the reference value for GAS6-AS1 gene expression may be determined at P3. In one embodiment, the reference value for GAS6-AS1 gene expression may be determined at P4. In one embodiment, the reference value for GAS6-AS1 gene expression may be determined at P5. In one embodiment, the reference value for GAS6-AS1 gene expression may be determined at P6. In one embodiment, the reference value for GAS6-AS1 gene expression may be determined at P7. In one embodiment, the reference value for GAS6-AS1 gene expression may be determined at P8. In one embodiment, the reference value for GAS6-AS1 gene expression may be determined at P9. In one embodiment, the reference value for GAS6-AS1 gene expression may be determined at P10.

A reference value for GAS6-AS1 gene expression may be obtained by the same techniques/method by which the level of GAS6-AS1 gene expression may be determined. Techniques for determining gene expression levels are described elsewhere in this description. In one embodiment, GAS6-AS1 gene expression levels may be determined by high throughput transcriptomic sequencing (a.k.a., RNA sequencing or RNA-seq) and optionally processed by various software packages as described in the Examples. In one embodiment, GAS6-AS1 gene expression levels may be determined by quantitative polymerase chain reaction (qPCR, a.k.a., real time PCR). In one embodiment, GAS6-AS1 gene expression levels may be determined by digital PCR (a.k.a., droplet digital PCR).

Suitably, the reference value for the level of GAS6-AS1 gene expression may be measured in units of gene counts, suitably relative gene counts. Suitably, wherein the gene counts are relative (i.e. normalised) to library size, suitably relative to a high throughput transcriptomic sequencing (a.k.a., RNA sequencing or RNA-seq) library size. Suitably the reference value for GAS6 gene expression may be measured in units of counts per million mapped reads (CPM) or reads per million mapped reads (RPM). Suitably gene counts are typically measured in counts per million mapped reads (CPM) or reads per million mapped reads (RPM).

Suitably the reference value for the level of GAS6-AS1 gene expression may be 10k (wherein ‘k’ denotes 1000 i.e. 10,000), 15k (15,000), 20k (20,000), 25k (25,000), 30k (30,000), 35k (35,000), or 40k (40,000) relative gene counts, suitably CPM. Suitably at any passage, such as (P1), P2, P3, P4, P5, P8, P10, P15, P20, P25, P30 and/or higher passage numbers, in one embodiment at passage 1 (P1). Suitably, the reference value for the level of GAS6-AS1 gene expression may be 16k, 17k, 18k, 19k, or 20k relative gene counts, suitably CPM. Suitably at any passage, such as (P1), P2, P3, P4, P5, P8, P10, P15, P20, P25, P30 and/or higher passage numbers, in one embodiment at P5. Suitably, the reference value for the level of GAS6-AS1 gene expression may be 11k, 15k, 20k, 25k, 30k, 35k, 40k, or 44k relative gene counts, suitably CPM. Suitably at any passage, such as (P1), P2, P3, P4, P5, P8, P10, P15, P20, P25, P30 and/or higher passage numbers, in one embodiment at passage 10. Suitably, the reference value for the level of GAS6-AS1 gene expression may be 20k, 25k, or 30k relative gene counts, suitably CPM. Suitably at any passage, such as (P1), P2, P3, P4, P5, P8, P10, P15, P20, P25, P30 and/or higher passage numbers, in one embodiment at P15. Suitably, the reference value for the level of GAS6-AS1 gene expression may be 20k relative gene counts, suitably CPM. Suitably at any passage number between, and including, P1 and P15. Suitably, the reference value for the level of GAS6-AS1 gene expression may be 20k relative gene counts, suitably CPM at any passage number.

Suitably therefore, the MSC has a high proliferative capacity if its expression level of the GAS6-AS1 gene is below 10k, 15k, 20k, 25k, 30k, 35k, or 40k relative gene counts, suitably CPM. Suitably at any passage, such as (P1), P2, P3, P4, P5, P8, P10, P15, P20, P25, P30 and/or higher passage numbers, in one embodiment at passage 1 (P1). Suitably therefore, the MSC has a high proliferative capacity if its expression level of the GAS6-AS1 gene is below 16k, 17k, 18k, 19k, or 20k relative gene counts, suitably CPM. Suitably at any passage, such as (P1), P2, P3, P4, P5, P8, P10, P15, P20, P25, P30 and/or higher passage numbers, in one embodiment at P5. Suitably therefore, the MSC has a high proliferative capacity if its expression level of the GAS6-AS1 gene is below 11k, 15k, 20k, 25k, 30k, 35k, 40k, or 44k relative gene counts, suitably CPM. Suitably at any passage, such as (P1), P2, P3, P4, P5, P8, P10, P15, P20, P25, P30 and/or higher passage numbers, in one embodiment at P10. Suitably therefore, the MSC has a high proliferative capacity if its expression level of the GAS6-AS1 gene is below 20k, 25k, or 30k relative gene counts, suitably CPM. Suitably at any passage, such as (P1), P2, P3, P4, P5, P8, P10, P15, P20, P25, P30 and/or higher passage numbers, in one embodiment at P15. Suitably therefore, the MSC has a high proliferative capacity if its expression level of the GAS6-AS1 gene is below 20k relative gene counts, suitably CPM at any passage number between, and including, P1 and P15. Suitably therefore, the MSC has a high proliferative capacity if its expression level of the GAS6-AS1 gene is below 20k relative gene counts, suitably CPM at any passage number.

Suitably the above reference values may be applicable when measuring the level of GAS6-AS1 gene expression using RNA-seq. Suitably therefore the above reference values are as measured using RNA-seq. Optionally one or more further processing steps may be applied to the gene expression data obtained from RNA-seq, such as genome alignment using Bowtie2 and an appropriate reference genome, sequence counts using htseq-count and R library DESeq2 rlog transformation. An exemplary method of measurement of GAS6-AS1 gene expression is shown in the examples herein.

The reference value for the ratio of the gene expression levels of GAS6-AS1:GAS6 (i.e., the ratio of GAS6-AS1:GAS6 gene expression levels) is suitably above the value of the ratio of GAS6-AS1:GAS6 gene expression that is found in those MSCs which have been determined as having high proliferative capacity, wherein high proliferative capacity has been established as described elsewhere herein. Suitably establishing whether a population has high proliferative capacity can be done by culturing said population and measuring one or more of the growth characteristics defined above.

In the same way, the reference value for the ratio of GAS6-AS1:GAS6 gene expression levels is suitably the same as or below the value of the ratio of GAS6-AS1:GAS6 gene expression found in MSCs which have been determined as having low proliferative capacity, wherein low proliferative capacity has been established as described elsewhere herein. Suitably establishing whether a population has low proliferative capacity can be done by culturing said population and measuring one or more of the growth characteristics defined above.

Suitably, the reference value for the ratio of gene expression levels of GAS6-AS1:GAS6 is higher than the ratio of GAS6-AS1:GAS6 gene expression of those MSCs which have been determined as having high proliferative capacity, wherein high proliferative capacity has been established as described elsewhere herein. Suitably establishing whether a population has high proliferative capacity can be done by culturing said population and measuring one or more of the growth characteristics defined above.

Suitably, the reference value for the ratio of the gene expression levels of GAS6-AS1:GAS6 may be above the value of the ratio of relative expression of GAS6-AS1:GAS6 found in MSCs which have been determined as having high proliferative capacity, wherein high proliferative capacity has been established as described elsewhere herein, and wherein the ratio of relative expression of GAS6-AS1:GAS6 is the ratio of GAS6-AS1:GAS6 expression relative to the expression level of a control gene. Suitably establishing whether a population has high proliferative capacity can be done by culturing said population and measuring one or more of the growth characteristics defined above.

Suitably, therefore the reference value for the ratio of the gene expression levels of GAS6-AS1:GAS6 is higher than the ratio of relative expression of GAS6-AS1 of those MSCs which have been determined as having high proliferative capacity, wherein high proliferative capacity has been established as described elsewhere herein, and wherein the ratio of relative expression of GAS6-AS1:GAS6 is the ratio of GAS6-AS1:GAS6 expression relative to the expression level of a control gene. Suitably establishing whether a population has high proliferative capacity can be done by culturing said population and measuring one or more of the growth characteristics defined above.

In one embodiment, the MSC has a high proliferative capacity if its ratio of the level of gene expression of GAS6-AS1:GAS6 is below the ratio of the level of gene expression of GAS6-AS1:GAS6 found in MSCs which have been determined as having low proliferative capacity, wherein low proliferative capacity has been established as described elsewhere herein. Suitably establishing whether a population has high proliferative capacity can be done by culturing said population and measuring one or more of the growth characteristics defined above.

A reference value for the ratio of the gene expression of GAS6-AS1:GAS6 may be determined at cell passage number 1 (P1), P2, P3, P4, P5, P8, P10, P15, P20, P25, P30 and/or higher passage numbers. Suitably, the reference value for the ratio of the gene expression of GAS6-AS1:GAS6 may be determined at P1. Suitably, the reference value for the ratio of the gene expression of GAS6-AS1:GAS6 may be determined during P1-P10. In one embodiment, the reference value for the ratio of the gene expression of GAS6-AS1:GAS6 may be determined at P1. In one embodiment, the reference value for the ratio of the gene expression of GAS6-AS1:GAS6 may be determined at P2. In one embodiment, the reference value for the ratio of the gene expression of GAS6-AS1:GAS6 may be determined at P3. In one embodiment, the reference value for the ratio of the gene expression of GAS6-AS1:GAS6 may be determined at P4. In one embodiment, the reference value for the ratio of the gene expression of GAS6-AS1:GAS6 may be determined at P5. In one embodiment, the reference value for the ratio of the gene expression of GAS6-AS1:GAS6 may be determined at P6. In one embodiment, the reference value for the ratio of the gene expression of GAS6-AS1:GAS6 may be determined at P7. In one embodiment, the reference value for the ratio of the gene expression of GAS6-AS1:GAS6 may be determined at P8. In one embodiment, the reference value for the ratio of the gene expression of GAS6-AS1:GAS6 may be determined at P9. In one embodiment, the reference value for the ratio of the gene expression of GAS6-AS1:GAS6 may be determined at P10.

A reference value for the ratio of gene expression of GAS6-AS1:GAS6 may be obtained by the same techniques/methods by which GAS6 and/or GAS6-AS1 gene expression level may be determined, and then calculating a ratio thereof as described hereinabove. Techniques for determining gene expression levels are described elsewhere in this description. In one embodiment, GAS6 and GAS6-AS1 gene expression levels may be determined by high throughput transcriptomic sequencing (a.k.a., RNA sequencing or RNA-seq) and optionally processed by various software packages as described in the Examples. In one embodiment, the ratio of gene expression of GAS6-AS1:GAS6 may be determined by quantitative polymerase chain reaction (qPCR, a.k.a., real time PCR). In one embodiment, the ratio of gene expression of GAS6-AS1:GAS6 may be determined by digital PCR (a.k.a., droplet digital PCR). The reference value for the ratio of gene expression of GAS6-AS1:GAS6 may be obtained by directly dividing the gene expression level of GAS6-AS1 by the gene expression level of GAS6. Suitably the ratio may be calculated without first normalising the expression levels of GAS6-AS1 and GAS6 to a normalisation factor, such as a control gene normally used for normalisation purposes in the art. Suitably the ratio is already a ‘relative’ measurement of GAS6-AS1 gene expression compared to GAS6 gene expression therefore normalisation is not required.

Suitably, the reference value for the ratio of gene expression of GAS6-AS1:GAS6 may be measured as a ratio of the relative gene counts, suitably CPM, of GAS6-AS1 to GAS6. Suitably wherein the relative gene counts of GAS6-AS1 and GAS6 are measured and expressed as described above. Suitably, measured and expressed as gene counts relative to library size, suitably relative to a high throughput transcriptomic sequencing (a.k.a., RNA sequencing or RNA-seq) library size. Suitably the reference value for the ratio of gene expression of GAS6-AS1:GAS6 may be measured as a ratio of the relative gene counts of GAS6-AS1 to GAS6 in units of counts per million mapped reads (CPM) or reads per million mapped reads (RPM). Suitably gene counts are typically measured in counts per million mapped reads (CPM) or reads per million mapped reads (RPM).

Suitably the reference value for the ratio of gene expression of GAS6-AS1:GAS6 may be 1:300, 1:350, 1:400, 1:450, 500, 1:550, or 1:600 at any passage, such as (P1), P2, P3, P4, P5, P8, P10, P15, P20, P25, P30 and/or higher passage numbers, in one embodiment at passage 1 (P1). Suitably, the reference value for the ratio of gene expression of GAS6-AS1:GAS6 may be 1:450, 1:500, 1:550, or 1:600 at any passage, such as (P1), P2, P3, P4, P5, P8, P10, P15, P20, P25, P30 and/or higher passage numbers, in one embodiment at P5. Suitably, the reference value for the ratio of gene expression of GAS6-AS1:GAS6 may be 1:550, 1:600, 1:650, or 1:700 at any passage, such as (P1), P2, P3, P4, P5, P8, P10, P15, P20, P25, P30 and/or higher passage numbers, in one embodiment at P10. Suitably, the reference value for the ratio of gene expression of GAS6-AS1:GAS6 may be 1:550, 1:600, or 1:650 units at any passage, such as (P1), P2, P3, P4, P5, P8, P10, P15, P20, P25, P30 and/or higher passage numbers, in one embodiment at P15. Suitably, the reference value for the ratio of gene expression of GAS6-AS1:GAS6 may be 1:600 at any passage number between, and including, P1 and P15. Suitably, the reference value for the ratio of gene expression of GAS6-AS1:GAS6 may be 1:600 at any passage number.

Suitably therefore, the MSC has a high proliferative capacity if its ratio of GAS6-AS1:GAS6 gene expression is below 1:300, 1:350, 1:400, 1:450, 1:500, 1:550, or 1:600. Suitably at any passage, such as (P1), P2, P3, P4, P5, P8, P10, P15, P20, P25, P30 and/or higher passage numbers, in one embodiment at passage 1 (P1). Suitably therefore, the MSC has a high proliferative capacity if its ratio of GAS6-AS1:GAS6 gene expression is below 1:450, 1:500, 1:550, or 1:600. Suitably at any passage, such as (P1), P2, P3, P4, P5, P8, P10, P15, P20, P25, P30 and/or higher passage numbers, in one embodiment at P5. Suitably therefore, the MSC has a high proliferative capacity if its ratio of GAS6-AS1:GAS6 gene expression is below 1:550, 1:600, 1:650, or 1:700. Suitably at any passage, such as (P1), P2, P3, P4, P5, P8, P10, P15, P20, P25, P30 and/or higher passage numbers, in one embodiment at P10. Suitably therefore, the MSC has a high proliferative capacity if its ratio of GAS6-AS1:GAS6 gene expression is below 1:550, 1:600, or 1:650. Suitably at any passage, such as (P1), P2, P3, P4, P5, P8, P10, P15, P20, P25, P30 and/or higher passage numbers, in one embodiment at P15. Suitably therefore, the MSC has a high proliferative capacity if its ratio of GAS6-AS1:GAS6 gene expression is below 1:600 at any passage number between, and including, P1 and P15. Suitably therefore, the MSC has a high proliferative capacity if its ratio of GAS6-AS1:GAS6 gene expression is below 1:600 at any passage number.

For the avoidance of doubt, a low ratio of GAS6-AS1:GAS6 gene expression means that the expression of GAS6-AS1 is less than the relative expression of GAS6. For example, a ratio of GAS6-AS1:GAS6 gene expression of 1:600 is below a ratio of 1:300, a ratio of GAS6-AS1:GAS6 gene expression of 1:100 is below a ratio of 1:10.

Suitably the above reference values may be applicable when measuring the level of GAS6 and GAS6-AS1 gene expression using RNA-seq. Suitably therefore the above reference values are as measured using RNA-seq. Optionally one or more further processing steps may be applied to the gene expression data obtained from RNA-seq, such as genome alignment using Bowtie2 and an appropriate reference genome, sequence counts using htseq-count and R library DESeq2 rlog transformation. An exemplary method of measurement of GAS6 gene expression and GAS6-AS1 gene expression is shown in the examples herein.

A skilled person will understand that the reference values for the GAS6 gene expression level, GAS6-AS1 gene expression level, and the ratio of the gene expression GAS6-AS1:GAS6 (i.e., the ratio of GAS6-AS1:GAS6 gene expression levels) will be dependent on the method or technique used to measure and obtain gene expression data (i.e., data indicative of gene expression, i.e., data indicative of gene expression levels). Suitably such methods include those described elsewhere herein, including quantitative polymerase chain reaction (qPCR, a.k.a., real time PCR), high throughput transcriptomic sequencing (a.k.a., RNA sequencing or RNA-seq), in vivo fluorescent nucleotide imaging, DNA/RNA microarray analysis, expressed sequence tag (EST) sequencing, Sanger sequencing of complementary DNAs (cDNAs), digital PCR (a.k.a., droplet digital PCR), and Northern blotting, amongst any other method that may be known in the art. Suitably, such methods include various ways of normalising gene counts known in the art, for example normalising with respect to sequencing library size, suitably measured in units of counters per million mapped reads (CPM) or reads per million mapped reads (RPM), or normalising with respect to sequencing library size and with respect to target transcript size wherein the target transcript corresponds to the gene being measured, suitably measured in units of reads per kilobase of transcript per million mapped reads (RPKM), fragments per kilobase of transcript per million mapped reads (FPKM), or transcripts per million (TPM). Therefore, as indicated above, the reference levels are expressed as relative gene counts, suitably gene counts relative to high throughput transcriptomic sequencing (a.k.a., RNA sequencing or RNA-seq) library size, suitably measured in units of counts per million mapped reads (CPM) or reads per million mapped reads (RPM), or which are compared to the level of expression of a control gene, measured using the same measurement method. Of course the exact units and gene expression levels measured may differ between different methods of measurement and data processing. As will be understood by a person skilled in the art, the invention is not limited to any particular method of measuring gene expression, or any particular units of measurement. The concept of the use of the expression of the GAS6 gene, the GAS6-AS1 gene or the ratio of expression thereof to determine MSCs with a high proliferative capacity applies in any case, when using any such method.

### Cell Identification, Selection, or Determination

Aspects of the invention relate to determining, identifying or selecting an MSC having high proliferative capacity, or distinguishing an MSC with high proliferative capacity from one with a low proliferative capacity.

It should be understood by a person skilled in the art that cells which have the ability to self-renew (i.e., self-replicate), through active growth and consequent cell division, divide into a population of cells. Suitably, selecting an individual MSC cell based on its traits such as high proliferative capacity may be interpreted as meaning the same thing as selecting a population of MSC cells which derive from said parent MSC cell having the relevant trait such as high proliferative capacity. Suitably therefore reference to an individual MSC herein encompasses references to a population of MSCs. Suitably the methods described herein may be interpreted as determining, identifying or selecting a population of mesenchymal stem cells having a high proliferative capacity, or distinguishing between a population of mesenchymal stem cells having a high proliferative capacity and a population of mesenchymal stem cells having a low proliferative capacity.

Suitably therefore the step of obtaining data indicative of the level of expression of GAS6 and/or GAS6-AS1 may be performed in one or more representative MSCs from the population. Suitably, steps which have been taken and which lead up to the determination, identification or selection of an MSC cell may be performed on any cell or group of cells from within the population of the MSC, and the subsequent cell or group of cells being selected may be another cell or group of cells from within the same population to the cell or group of cells which have been measured. A person skilled in the art will understand that methods of gene expression level determination, which the present invention teaches as being necessary to determine an MSC as having high or low proliferative capacity, can result in the destruction of the individual cell being measured. Suitably, a skilled person will understand that once a cell has been determined, selected, identified, or distinguished as having high proliferative capacity through measuring its gene expression levels, and whereby that cell has been destroyed in this process, the determination, selection, identification, or distinguishing may be directed towards another MSC of the same population as the MSCs which has been measured and destroyed.

### Pharmaceutical Compositions

The invention further provides a pharmaceutical composition comprising a population of mesenchymal stem cells of the invention having a high proliferative capacity.

Pharmaceutical compositions and formulations generally include one or more optional pharmaceutically acceptable carrier or excipient. The pharmaceutical composition may include at least one additional active pharmaceutical ingredient or therapeutic agent.

A “pharmaceutical composition” refers to a preparation which is in such form as to permit the biological activity of one or more active ingredients contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition. Carriers are described, e.g., by Remington's Pharmaceutical Sciences 16th edition². Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001 to about 4% by weight of the total composition. Methods for preparing administrable pharmaceutical compositions are known. Exemplary methods are described in more detail in, for example, Remington: The Science and Practice of Pharmacy³.

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent a relevant disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. For example, pharmaceutical compositions containing MSCs may be administered via localized injection, including catheter administration, systemic injection, localized injection, intravenous injection, or parenteral administration. When administering a therapeutic composition (e.g., a pharmaceutical composition containing cells of the invention), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### Combinations

According to another aspect of the present invention, the MSCs of the invention or populations thereof, may be combined with one or more further therapeutic agents to provide a combination therapy.

Suitably the combination may be provided as a composition comprising an MSC of the invention or a population thereof and a further therapeutic agent. Suitably the composition may be a pharmaceutical composition as described above.

Suitably the therapeutic agent may be any agent known for the prevention or treatment of a disease. Suitably the therapeutic agent is known for the prevention or treatment of the same disease as the disease which the MSCs are intended to prevent or treat. Suitably therefore, the combination is for use as a medicament, in the prevention or treatment of a disease. Suitable such diseases are described below.

Suitably the therapeutic agent and the MSC may have a synergistic effect in treating or preventing a disease. Suitably therefore the MSC may enhance the effect of the therapeutic agent, or vice versa.

Suitably the therapeutic agent may be a small chemical molecule or entity, a small molecule drug or pro-drug, a protein or peptide such as an enzyme or antibody, an oligonucleotide or a nucleic acid such as DNA or RNA (for example, mRNA, siRNA, miRNA), or a virus for example. Suitably the therapeutic agent may be selected from: tumor necrosis factor-related apoptosis-inducing ligand (TRAIL), chemotherapeutic drugs such as gemcitabine (GCB), paclitaxel (PTX), and doxorubicin (DOX), prodrugs such as 5-fluorocytosine (5-FC) and ganciclovir (GCV), immune cell-activating cytokines, and oncolytic virus.

Suitably the MSCs of the invention are useful for the delivery of the therapeutic agent. Suitably the MSCs of the invention may enhance or promote the therapeutic effect of the therapeutic agent. Suitably therefore, a further aspect of the invention provides the use of an MSC of the invention for delivery of a therapeutic agent, suitably any therapeutic agent described herein. Suitably therefore, the MSCs of the invention may act as vectors to deliver therapeutic agents to a target site, suitably for use in the treatment or prevention of a disease in a subject. Suitably the target site may be the site of a disease, diseased tissue, or cause of a disease. Suitable means of using MSCs as delivery agents are described in Hassanzadeh et al. Frontiers in Cell and Developmental Biology, Volume 9, 12 Jul. 2021.

In one embodiment, there is provided a composition comprising an MSC of the invention or a population thereof and a further therapeutic agent for use in the treatment or prevention of a disease, wherein the MSC delivers the therapeutic agent to a target site.

Advantageously, MSCs are particularly useful for this due to their innate homing ability to travel towards sites which may require treatment, such as sites of damaged tissue or inflammation. Furthermore, MSCs with high proliferative capacity can be produced faster at a larger scale and uniformity suitable for manufacturing therapeutic agents.

Suitably in some embodiments, use of the MSCs of the invention as delivery vectors may be achieved by formulating a composition of the MSCs of the invention and the therapeutic agent as mentioned above, suitably whereby the therapeutic agent may be in free form or encapsulated. Suitably the therapeutic agent may be encapsulated into for example nanocompartments, nanoshells, nanoparticles, liposomes, exosomes etc.

In some embodiments, the therapeutic agent may be associated with the MSCs. Suitably the therapeutic agent may be present within or attached to the MSCs. Suitably the therapeutic agent may be tethered to the MSCs, suitably to the cell membrane of the MSCs. Suitably the therapeutic agent may be tethered to the cell membrane intracellularly or extracellularly. Suitably the therapeutic agent may be tethered to the cell membrane by being attached to a membrane bound protein, suitably by being fused to a membrane bound protein. Suitably therefore the MSC may comprise a fusion protein, wherein the fusion protein comprises a therapeutic agent and a cellular protein, for example a membrane bound protein.

In one embodiment, therefore, there is provided an MSC having a high proliferative capacity, wherein level of expression of the GAS6 gene, GAS6-AS1 gene and/or the ratio of GAS6-AS1:GAS6 gene expression levels in the mesenchymal stem cell is below a reference value, and wherein the MSC comprises a therapeutic agent. In one embodiment, there is provided said MSC for use in the prevention or treatment of a disease in a subject.

However, in some embodiments, the therapeutic agent may also be produced by the MSCs of the invention. Suitably therefore the MSCs of the invention may be modified such that they are capable of production of the therapeutic agent. Suitably the MSCs may be modified to comprise one or more nucleic acid sequences encoding the therapeutic agent or means to produce the therapeutic agent, such as one or more biosynthetic enzymes.

In one embodiment, therefore, there is provided an MSC having a high proliferative capacity, wherein level of expression of the GAS6 gene, GAS6-AS1 gene and/or the ratio of GAS6-AS1:GAS6 gene expression levels in the mesenchymal stem cell is below a reference value, and wherein the MSC comprises one or more nucleic acid sequences encoding a therapeutic agent. In one such embodiment, the MSC is regarded as a modified MSC. In one embodiment, there is provided a population of said MSCs.

Suitably the MSCs may be modified by transformation of one or more nucleic acid sequences encoding the therapeutic agent or means to produce the therapeutic agent such as one or more enzymes. Suitably the one or more nucleic acids may be comprised upon an expression construct, optionally upon a vector, operable to express the or each nucleic acid in the MSC. Methods that are well known to those skilled in the art can be used to construct expression vectors containing coding sequences for a therapeutic agent or means to produce the therapeutic agent, and appropriate transcriptional and translational control signals.

Suitably the expression construct or vector may be transformed into the MSC. Suitable means of transformation of cells are known in the art. The term “vector” or “expression vector” is used herein to mean a vehicle for introducing into and expressing a desired gene in a host cell. As known to those skilled in the art, such vectors may easily be selected from the group consisting of plasmids, phages, viruses and retroviruses. In general, vectors will comprise a selection marker, appropriate restriction sites to facilitate cloning of the desired gene and the ability to enter and/or replicate in MSCs. For the purposes of this disclosure, numerous expression vector systems may be employed. Of course, any expression vector that is capable of eliciting expression in eukaryotic cells may be used in the present disclosure. Examples of suitable vectors include, but are not limited to plasmids pcDNA3, pHCMV/Zeo, pCR3.1, pEF 1/His, pIND/GS, pRc/HCMV2, pSV40/Zeo2, pTRACER-HCMV, pUB6/V5-His, pVAXI, and pZeoSV2 (available from Invitrogen, San Diego, Calif.), and plasmid pCI (available from Promega, Madison, Wis.).

Suitably, once the vector or construct encoding the therapeutic agent or means to produce the therapeutic agent has been prepared, the expression vector or construct may be introduced into the MSCs. Introduction of into the MSCs can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway (1988) “Mammalian Expression Vectors” in Vectors, ed. Rodriguez and Denhardt (Butterworths, Boston, Mass.), Chapter 24.2, pp. 470-472. Typically, vector introduction into the MSC is via electroporation.

Suitably the expression construct or vector may comprise one or more control or regulatory elements operably linked to the or each nucleic acid. Suitable regulatory elements may include promoters, enhancers, UTRs, introns, etc. Suitably the expression construct or vector at least comprises a promoter which is suitable to promote expression of the one or more nucleic acids in the MSC. Suitably the promoter may be an inducible promoter, which may be induced upon contact with an inducer, suitably the promoter may be induced after the MSC has been administered to a subject to therefore promote expression of the or each nucleic acid in the subject.

The MSCs harbouring the expression vector or construct may then suitably be grown or cultured under conditions appropriate to the production of the therapeutic agent, and may suitably be assayed for protein synthesis. Exemplary assay techniques include enzyme-linked immunosorbent assay (ELISA), radioimmunoassay (RIA), or fluorescence-activated cell sorter analysis (FACS), immunohistochemistry and the like. Suitably such culturing and assaying steps may take place before the MSCs are used in the treatment of a subject. Suitably only those MSCs producing the therapeutic agent are then used for treatment of subject.

For long-term, high-yield production of recombinant proteins, stable expression may be preferred. For example, cell lines that stably express the therapeutic protein may be engineered. Rather than using expression vectors that contain viral origins of replication, MSCs can be transformed with DNA controlled by appropriate expression control elements (e.g., promoter, enhancer, sequences, transcription terminators, polyadenylation sites, etc.), and a selectable marker. Following the introduction of the foreign DNA, engineered MSCs may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer MSC lines which stably express the therapeutic agent or means to produce the therapeutic agent.

Suitably, the expression vector or construct is transferred to MSCs by conventional techniques, and the transfected MSCs may then be administered to a subject. Suitably the MSCs may then produce the therapeutic agent in vivo within the subject. Therefore, the present invention includes MSCs as described herein further containing a polynucleotide encoding a therapeutic agent. Suitably which may be regarded as modified MSCs.

Suitably said modified MSCs may be for use as a medicament. Suitably said modified MSCs may be for use in the treatment or prevention of a disease as described herein. Suitably said modified MSCs may treat or prevent disease in a subject by producing the therapeutic agent in vivo.

In one embodiment, there is provided a culture medium comprising MSCs of the invention. In one embodiment there is provided a fermentation vessel comprising said culture medium.

Suitably the MSCs may further secrete the therapeutic agent. Suitably therefore the one or more nucleic acids encoding the therapeutic agent may comprise a nucleic acid sequence encoding one or more targeting peptides. Suitably the one or more targeting peptides may mediate transport of the therapeutic agent out of the MSC. Suitably, the one or more targeting peptides may additionally or alternatively mediate transport of the therapeutic agent into a specific type of cell, suitably for the treatment or prevention of disease. Suitably therefore the one or more nucleic acids encoding the therapeutic agent may comprise a nucleic acid sequence encoding a targeting peptide which mediates transport of the therapeutic agent out of the MSC and a targeting peptide which mediates transport of the therapeutic agent into a specific type of cell, suitably a target cell, suitably for the treatment or prevention of disease.

### Use of MSCs and Pharmaceutical Compositions Thereof as Medicaments

According to further aspects of the invention, populations of MSCs of the invention and/or combinations of said MSCs with other therapeutic agents, and/or pharmaceutical compositions thereof may be used as medicaments, or may be formulated for use as medicaments, and are referred herein as “medicaments of the invention”.

Suitably the medicaments of the invention may be used to prevent or treat a disease in a subject. Suitably the medicaments of the invention thereof may be used to prevent or treat any diseases by virtue of the sustained trophic repair activity of MSCs, and/or by virtue of the ability of MSCs for multipotent differentiation including for example chondrogenesis, osteogenesis, adipogenesis, and angiogenesis.

Suitably the medicaments of the invention may be used to prevent or treat a disease of the musculoskeletal system, a disease of the immune system, a disease of the endocrine system, a disease of the cardiovascular system, a disease of the skin, a disease of the nervous system, or a disease of the respiratory system, or any combination thereof in a subject.

Suitably the medicaments of the invention thereof may be used to prevent or treat a bone or soft tissue disease or injury, a liver disease or injury, a bowel disease or injury, a lung disease or injury, a skin disease or injury, a heart disease or injury, a kidney disease or injury, a uterus disease or injury, or any combination thereof in a subject.

Suitably the medicaments of the invention thereof may be used to prevent or treat an autoimmune disease, a fibrotic disease, an inflammatory disease, an epithelial disease in a subject.

Autoimmune diseases suitably treated by the medicaments of the invention include, but are not limited to, lupus, Type 1 diabetes, multiple sclerosis, uveitis, autoimmune thyroid disease, scleroderma, Graves' Disease, Crohn's disease, autoimmune lymphoproliferative disease, inflammatory bowel disease, ulcerative colitis, demyelinating disease, autoimmune encephalomyelitis, autoimmune gastritis, rheumatoid arthritis, and autoimmune glomerular diseases.

Suitably the medicaments of the invention may be used to prevent or treat one or more of the following in a subject:
a bone or soft tissue disease or injury, suitably selected from: osteoarthritis, meniscus cartilage injury (such as torn meniscus), ligament injury (such as torn ligament), a wound, skin injury, bone injury, and cartilage injury;
a liver disease or injury, suitably selected from: liver cirrhosis, decompensated liver cirrhosis, liver failure caused by Hepatitis B or C virus, primary biliary cirrhosis, alcoholic cirrhosis, non-alcoholic fatty liver disease, end stage liver disease with Hepatitis C, liver allograft rejection, acute-on-chronic liver failure, and autoimmune induced liver cirrhosis;
an autoimmune disease, suitably selected from: rheumatoid arthritis, type 1 diabetes, multiple sclerosis, systemic lupus erythematosus, vasculitis, and inflammatory bowel disease;
a fibrotic disease, suitably selected from fibrosis of the lungs (such as idiopathic pulmonary fibrosis, radiation therapy induced fibrosis, hypersensitivity pneumonitis, fibrosis of the liver), fibrosis of the heart (such as myocardial infarction, aortic stenosis, coronary heart disease genetic related cardiomyopathy), fibrosis of the kidney (such as chronic glomerulonephritis, diabetic nephropathy, hypertensive nephropathy and chronic renal allograft injury), fibrosis of the uterus, and fibrosis of the skin.

In one embodiment, the medicaments of the invention may be used to prevent or treat a bone or soft tissue disease or injury, suitably osteoarthritis in a subject.

Suitably the disease may be acquired or may be inherited, in some cases the disease may be a genetic disease.

Suitably MSCs of the invention, or populations thereof, may be subject to genetic manipulation prior to use as a therapeutically active agent, for example to improve the efficiency with which they target themselves to sites of inflammation, or to increase the potency of therapeutic action for a particular indication.

Medicaments may take any shape or form such that they are able to fulfil their therapeutically relevant purpose as medicaments. It is to be understood that the scope of the present invention is not to be limited to the treatment of the specific diseases mentioned herein.

Suitably a subject as defined herein may also be referred to as a ‘patient’. Suitably the subject may be a mammalian subject, including humans; domestic animals; farm animals; such as dogs, cats, guinea pigs, rabbits, rats, mice, horses, cattle, cows, etc. Suitably the subject is human. Suitably the subject may be undergoing medical care, or requesting medical care. Suitably the subject is male or female. Suitably the subject is an adult or a child.

Suitably the subject may be one believed to have any disease as mentioned hereinabove, or diagnosed with a disease as mentioned above. For example, a suitable subject may have symptoms consistent with such diseases.

Alternatively, a suitable subject may be one believed to be at risk of developing such a disease. For example, a suitable subject may have been in contact with an individual suffering from such a disease, may suffer from a related disease, or may satisfy risk factors associated with said conditions like smoking, old age, allergy etc.

### Method of Preparing a Population of MSCs

A skilled person will be aware of techniques in the art for culturing MSCs, and MSC culturing techniques are also described in the Examples. Generally, culturing of MSCs, like any other cell, requires exposing the cell to appropriate conditions that are tolerated by the cell and which allow it to maintain its normal cellular physiology, including exposing the cell to appropriate nutrients which are required by the cell through its normal course of metabolism to survive and grow. Nutrient exposure may be achieved by culturing the cells in either liquid nutrient medium, or on solid nutrient medium, under appropriate conditions of temperature, humidity, atmospheric gas concentrations and pressures and other parameters.

Suitable nutrients for culturing a population of MSCs may include: Dulbecco's Modified Eagles Medium supplemented with 10% (v/v) foetal bovine serum, 1% (v/v) Glutamax (Sigma) and optionally 1% (v/v) penicillin/streptomycin, optionally supplemented with 10 ng/ml FGF-2 (Peprotech) to aid in maintaining cells in an undifferentiated state.

Suitable conditions of temperature, humidity, atmospheric gas concentrations and pressures and other parameters for culturing a population of MSCs may include: 37° C., a humidified atmosphere, 5% atmospheric CO₂, and 95% atmospheric air.

The first and eleventh aspects of the invention provides a method which includes selecting a population of cells for MSCs in which level of expression of the GAS6 gene, GAS6-AS1 gene and/or the ratio of GAS6-AS1:GAS6 gene expression levels is below a reference value.

A skilled person will understand the term “selecting” as used in the context of cell populations. Selection may be achieved by positive selection of desired cells (i.e. those with high proliferative capacity) or by negative selection i.e. elimination of undesired cells (i.e. those with low proliferative capacity).

Suitably selecting may comprise separating the desired cells from the undesired cells. Suitably selecting may comprise separating the MSCs in which expression level of the GAS6 gene, GAS6-AS1 gene and/or the ratio of GAS6-AS1:GAS6 gene expression levels is below a reference value i.e. which have high proliferative capacity. Suitably separating from the rest of the cells in the population, suitably from the MSCs in which the expression level of the GAS6 gene, GAS6-AS1 gene and/or the ratio of GAS6-AS1:GAS6 gene expression levels is above a reference value i.e. which have low proliferative capacity.

Suitably the method may comprise a step of expanding the selected cells. Suitably expanding the selected cells may comprise culturing the selected cells under suitable conditions to cause growth or proliferation. Suitably expanding the cells may be carried out until the population of cells is increased to the desired size, for example for 5, 10, 15, 20, 25, 30, 35 generations or more.

The eleventh aspect of the invention may comprise a final step of formulating the MSCs having a high proliferative capacity into a medicament, suitably for use in therapy as described elsewhere herein. Suitably formulating the MSCs comprises forming a pharmaceutical composition comprising the MSCs. Suitable formulations for such pharmaceutical compositions comprising cells are described above.

### BRIEF DESCRIPTION OF THE DRAWINGS

FIG. 1: the relative GAS6 gene expression (i.e., relative gene count in CPM) at passage 1 in the two fastest growing (high proliferation capacity) and two slower growing (low proliferation capacity) human bone marrow-derived MSCs obtained from four patient samples. Results are shown as the mean and standard deviation.

FIG. 2: the relative GAS6-AS1 gene expression (i.e. relative gene count in CPM) at passage 1 in the two fastest growing (high proliferation capacity) and two slower growing (low proliferation capacity) human bone marrow-derived MSCs obtained from four patient samples. Results are shown as the mean and standard deviation.

FIG. 3: the gene expression ratio of GAS-AS1 over GAS6 (i.e., the ratio of the relative gene count in CPM of GAS-AS1:GAS6) at passage 1 in the two fastest growing (high proliferation capacity) and two slower growing (low proliferation capacity) human bone marrow-derived MSCs obtained from four patient samples. Results are shown as the mean and standard deviation.

FIG. 4: relative GAS6 gene expression (i.e. relative gene count in CPM) at various timepoints as measured by the number of passages (P1, P5, P10, and P15) in the two fastest growing (high proliferation capacity) and two slower growing (low proliferation capacity) human bone marrow-derived MSCs obtained from four patient samples. Dotted line indicates a reference value of 45. Results are shown as the mean and standard deviation.

FIG. 5: relative GAS6-AS1 gene expression (i.e. relative gene count in CPM) at various timepoints as measured by the number of passages (P1, P5, P10, and P15) in the two fastest growing (high proliferation capacity) and two slower growing (low proliferation capacity) human bone marrow-derived MSCs obtained from four patient samples. Dotted line indicates reference value: 20,000. Results are shown as the mean and standard deviation.

FIG. 6: the gene expression ratio of GAS-AS1 over GAS6 (i.e., the ratio of the relative gene count in CPM of GAS-AS1:GAS6) at various timepoints as measured by the number of passages (P1, P5, P10, and P15) in the two fastest growing (high proliferation capacity) and two slower growing (low proliferation capacity) human bone marrow-derived MSCs obtained from four patient samples. Dotted line indicates reference value: 600. Results are shown as the mean and standard deviation.

FIG. 7: Growth and phenotypic characteristics of mesenchymal stem cells (MSCs) from four patients (PN241, PN242, PN251, PN264). A) The percentage of cells expressing MSC markers CD90 and CD105 and hematopoietic stem cell markers CD34 and CD45 were determined by fluorescence-activated cell sorting. B) The number of population doublings (PD) reached after each passage using MSCs from each patient was recorded until growth ceased (for donor PN242, data were collected until passage 30 until which point the cells continued to grow). C) The population doubling time (PDT) is shown up to passage 16 for all four patients.

### EXAMPLES

Certain embodiments of the present disclosure now will be illustrated by the following Examples. The present disclosure may, however, be embodied in many different forms and should not be construed as limited to the embodiments set forth herein; rather, these embodiments are provided so that this disclosure will be thorough and complete, and will fully convey the scope of the disclosure to those skilled in the art.

### Isolation and Expansion of Human Marrow Derived MSCs for In Vitro Studies

The model used as the basis for all the experiments reported here was long-term culture of human bone-marrow derived MSCs. Bone marrow plugs were collected from the femoral heads of patients undergoing total hip replacement. All patients gave their informed consent and the study was carried out according to local ethical guidelines (North Bristol NHS Trust Research Ethics Committee). Patient details can be seen in Table 1. Cells were suspended in stem cell expansion medium consisting of low glucose Dulbecco's Modified Eagles Medium (Sigma) supplemented with 10% (v/v) Foetal Bovine Serum (FBS, Thermo Scientific Hyclone, Loughborough, UK), 1% (v/v) Glutamax (Sigma) and 1% (v/v) Penicillin/Streptomycin (Sigma). The serum batch was selected to promote the growth and differentiation of MSCs (Kafienah et al., 2007a). The medium was also supplemented with 10 ng/ml FGF-2 (Peprotech). This growth factor has been previously shown to enhance the MSC proliferation rate in vitro^{4, 5}, to retain MSCs as undifferentiated cells during proliferation^{6, 7} and to enhance chondrogenic differentiation when the FGF-2 expanded MSCs are subsequently exposed to differentiation conditions^{4, 5}. The cell suspension was separated from any bone in the sample by repeated washing with media. The cells were centrifuged at 500 g for 5 minutes and the supernatant/fat removed. The resulting cell pellet was resuspended in medium, and then plated at a seeding density of between 1.5-2.0×10⁵ nucleated cells per cm². These flasks were incubated at 37° C. in a humidified atmosphere of 5% CO₂ and 95% air. Four days were allowed before the first medium change and then the medium was changed every other day until adherent cells reached 90% confluence and were ready for passaging.

TABLE 1
Patient (i.e., donor) and MSC culture details. Bone marrow
was obtained from each of four donors undergoing surgery
following traumatic injury. Growth arrest was recorded
as the last passage at which an increase in cell number
was recorded. Cells from donor PN242 continued to proliferate
at P30, after which the experiment was terminated.

| | Age at surgery | | MSC passage at |
|---|---|---|---|
| Donor | (Years) | Gender | growth arrest |
| PN241 | 45 | M | P25 |
| PN242 | 38 | M | >P30 |
| PN251 | 70 | M | P17 |
| PN264 | 43 | M | P16 |

### Detection of Cell-Surface Phenotypic Markers

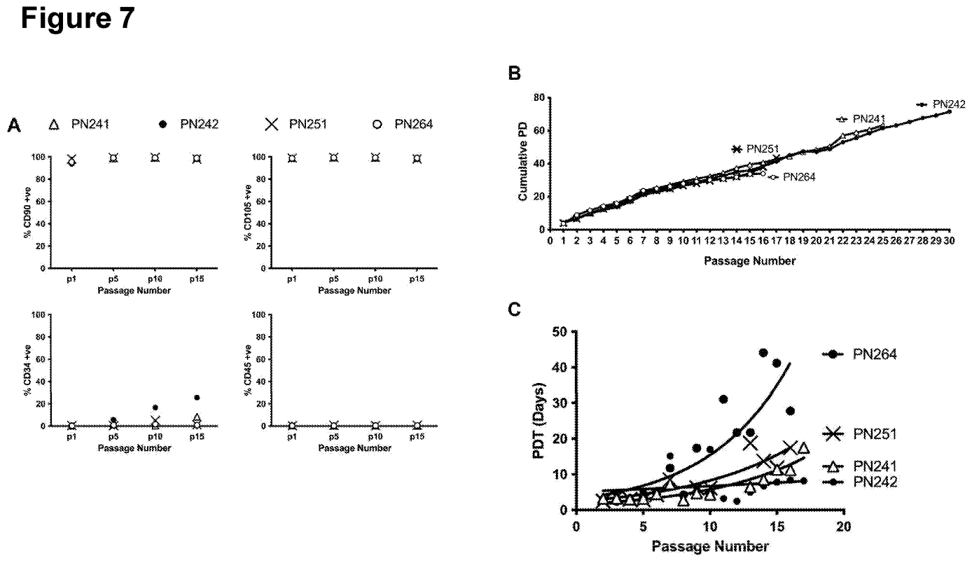

In order to confirm the MSC phenotype of cultured cells, high surface expression of CD90 and CD105 MSCs and low expression of CD34 and CD45 was established. MSCs (100,000 cells from each patient at each of passages 1, 5, 10 and 15) were suspended in a 1:500 dilution of Zombie (Biolegend), a live/dead cell dye, and incubated for 20 mins in the dark. Nonspecific antigens were then blocked by incubating the cells at room temperature for 1 hour in 1% (weight/vol) BSA (Sigma-Aldrich), 5% (vol/vol) FCS (Sigma-Aldrich), and 10% (vol/vol) human serum (Sigma-Aldrich). The cells were washed by centrifugation in three volumes of PBS, and the cell pellet was suspended in 100 μl of a primary antibody solution containing 20-100 μg/ml of antibody in blocking solution. All the primary antibodies were fluorescent-labelled mouse anti-human IgGs: anti-CD105-fluorescein isothiocyanate (FITC), anti-CD90-phycoerythrin (PE), anti-CD45-PE were from R&D Systems); anti-CD34-FITC was from BD Bioscience; IgG1-FITC and IgG1-PE isotype controls were from R&D Systems. After incubation for 40 minutes at 4° C., the cells were washed and suspended in 1 ml of PBS for analysis on a Canto flow cytometer (BD FACSCanto II), after the exclusion of non-viable cells. Data were analysed using FlowJo (Treestar). Positive expression was defined as the level of fluorescence greater than 95% of corresponding isotype-matched control antibodies. The results are can be seen in FIG. 7 and confirm the identity of cultured cells as being consistent with MSCs.

### Cell Passaging and Calculation of Population Doublings and Doubling Time

At the end of each passage the MSCs were harvested using 0.25% trypsin-EDTA (Invitrogen), pooled, counted and then divided into different centrifuge tubes for reseeding and further growth, for storage in liquid nitrogen for subsequent use for genomic analysis. The cells for each patient were passaged continuously without freezing, until growth arrest, defined as no detectable increase in cell number between passages (see Table 1 and FIG. 7B). At each passage, the total number of harvested MSCs was determined. The first cell harvest after seeding of fresh bone marrow was taken as passage 0. The number of cells reseeded at the start of Passage 1 was used as the baseline for calculation of the first population doubling value at the end of passage 1. Downstream analyses of the MSCs were undertaken from passage 1 onwards.

The number of Population doublings (PDs) i.e. length of growth phase was calculated using the following formula:

$$PDs = [ log ( Number of Harvested MSCs ) - log ( Number of seeded MSCs ) ] / log ( 2 ) ]$$

The PD for each passage was calculated and added to the PD of the previous passages to generate data for Cumulative PD at each Passage.

Population doubling time (PDT) i.e. rate of growth was calculated for each passage using the formula:

$$PDT = t \times log ( 2 ) / log ( cells harvested / cells seeded )$$

$$( t = the time between cell seeding and cell harvesting )$$

Changes in cumulative PD and PDT with passage number for each patient can be seen in FIG. 7.

### Acquisition of Genomic Data

### Transcriptomics

Transcriptomics was performed by the Centre for Genomic Research on mRNA extracted from the mRNA of all four patients at P1, P5, P10 and P15. When MSCs were harvested at the end of each passage, 1×10⁶ cells were isolated and resuspended in RNAprotect Cell Reagent (Qiagen). The cells were stored at −80° C. until the complete set of samples from all donors and time points had been collected. RNA was then extracted from selected time points using the RNeasy Plus Mini Kit (Qiagen), according to the manufacturer's instructions. The concentration of RNA in the extract was determined using a NanoDrop 2000 spectrophotometer (Thermo). Extracted RNA was stored at −80° C. prior to analysis.

Ribosomal RNA depletion was performed using the Ribo-Zero™ H/M/R Kit (Illumina) and RNASeq libraries were then prepared using the NEB Next Ultra Directional RNA Library Prep Kit (Illumina). Paired-end sequencing of the RNASeq libraries was performed by the Illumina HiSeq4000 platform using V4 chemistry.

### Quantification and Statistical Analysis

Data processing, integration and analyses were undertaken by the Computational Biology Facility at the University of Liverpool. RNASeq data was acquired as described above. The raw Fastq files were trimmed for the presence of Illumina adapter sequences using Cutadapt version 1.2.1, option -O 3. Reads were further trimmed using Sickle version 1.200 with a minimum window quality score of 20. Reads shorter than 10 base pairs after trimming were removed. Sequence quality metrics were assessed using FastQC version 0.11.4. No samples were removed. Sequence data were aligned to the human genome version GRCh38 from NCBI using Bowtie2 version 1.1.2 with recommended parameters⁸. Gene level count data were generated from the Bowtie2 alignments using htseq-count version 0.9.0. The R library DESeq2 was used to produced rlog transformed count data. These were filtered to remove genes with less than 1 average count. Statistical analyses were performed in R version 3.4.4 and graphical representations were done using the R package ggplot2.

### Selection of Genomic Markers Related to Cell Growth

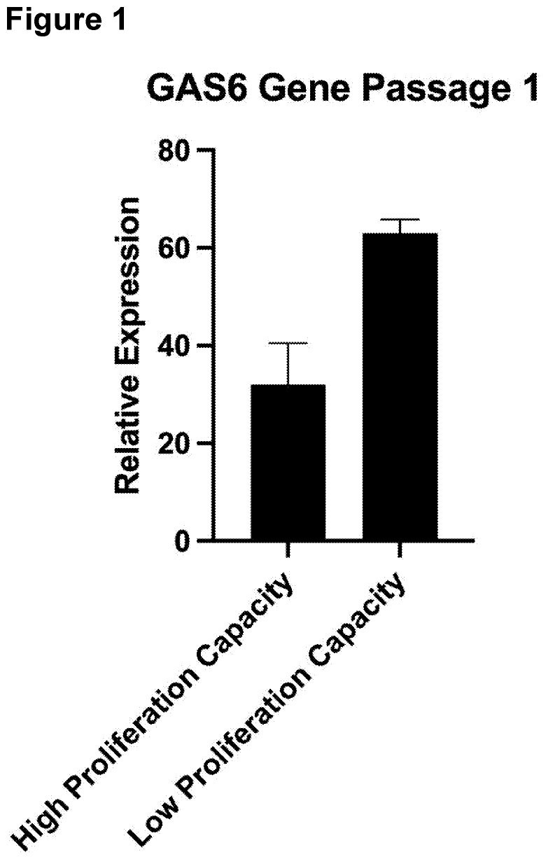

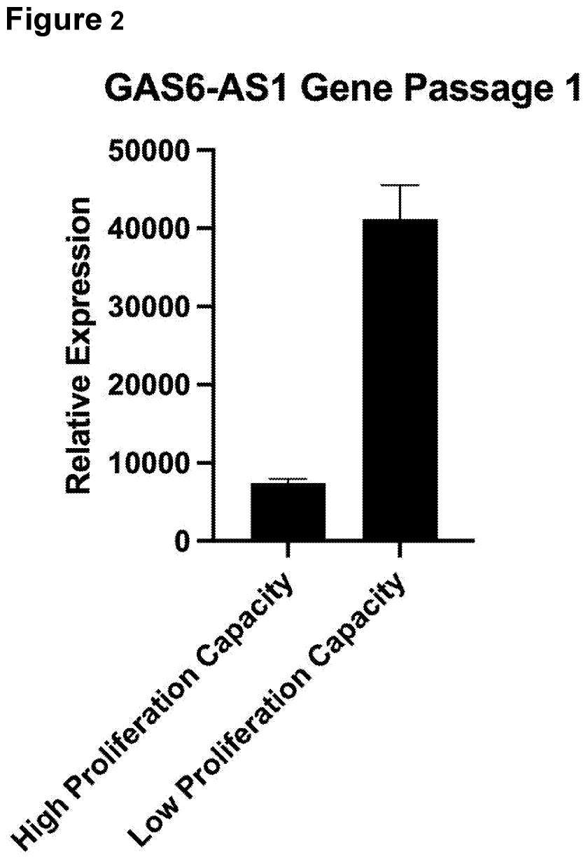

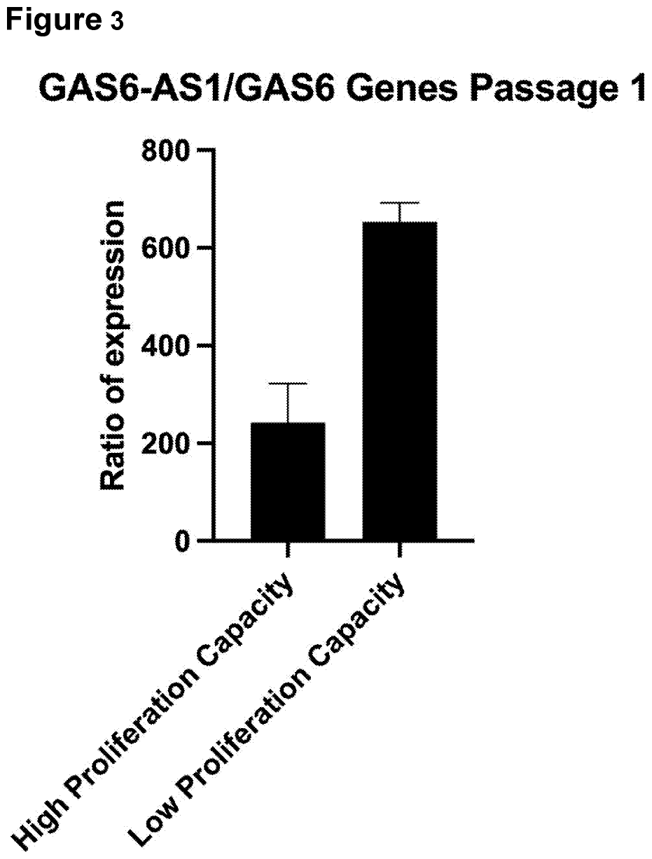

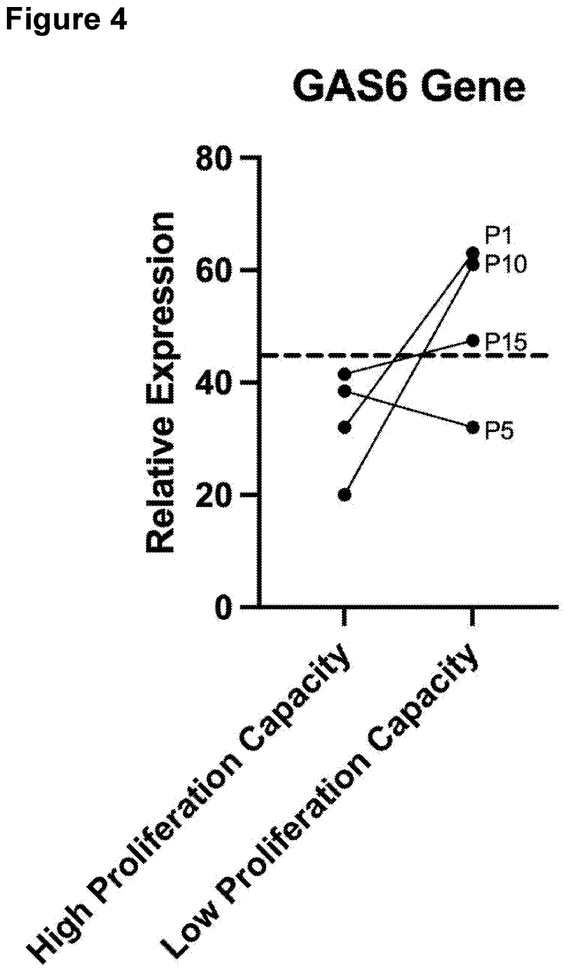

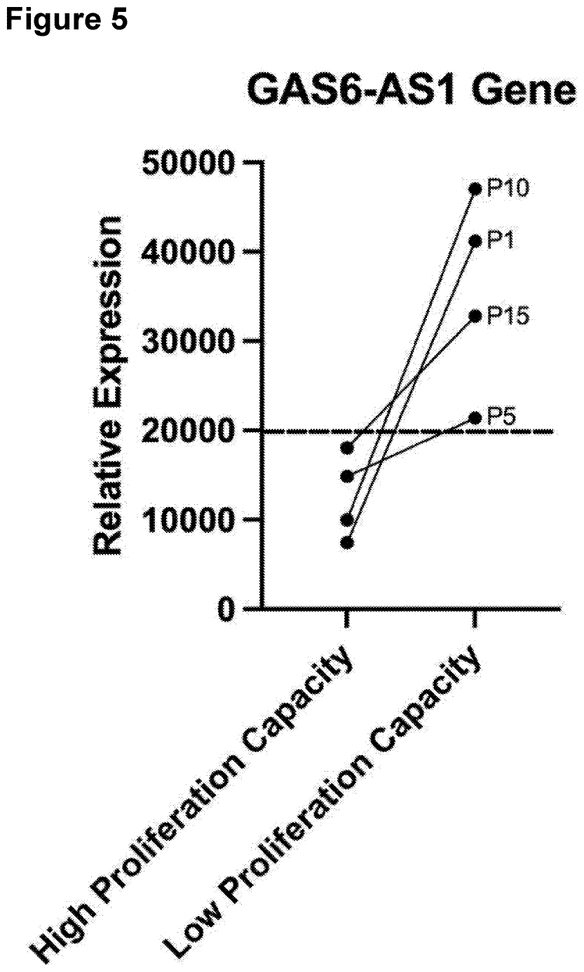

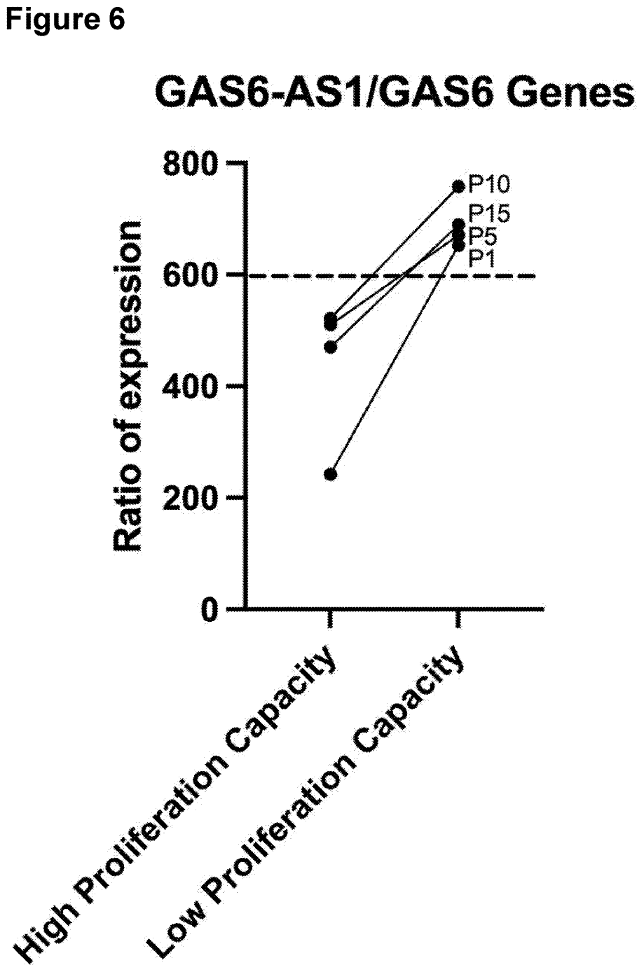

The dataset described above was analysed for any genes with a substantially different expression level at Passage 1 when comparing MSCs from patients PN241 and PN242 (high proliferative capacity samples) with MSCs from patients PN251 and PN264 (low proliferative capacity samples). Where genes were identified as being different, the next step was to demonstrate that the difference was consistently maintained at P5, P10 and P15. This analysis led to the selection of GAS6 and GAS6-A1 as biomarkers of high proliferative capacity in MSCs, as shown in FIGS. 1-6.

### REFERENCES

[1] Salerno A, Brady K, Rikkers M, Li C, Caamano-Gutierrez E, Falciani F, Blom A W, Whitehouse M R, Hollander A P: MMP13 and TIMP1 are functional markers for two different potential modes of action by mesenchymal stem/stromal cells when treating osteoarthritis. Stem Cells 2020, 38:1438-53.
[2] Remington J P, Osol A: Remington's Pharmaceutical sciences. 16th ed. Easton PA: Mach Publishing Co, 1980.
[3] Remington J P, Pharmaceutical P, Remington J P, Beringer P, University of the Sciences in P: Remington: the science and practice of pharmacy. 21st ed. Philadelphia: Lippincott Williams & Wilkins, 2005.
[4] Bianchi G, Banfi A, Mastrogiacomo M, Notaro R, Luzzatto L, Cancedda R, Quarto R: Ex vivo enrichment of mesenchymal cell progenitors by fibroblast growth factor 2. Exp Cell Res 2003, 287:98-105.
[5] Solchaga L A, Penick K, Porter J D, Goldberg V M, Caplan A I, Welter J F: FGF-2 enhances the mitotic and chondrogenic potentials of human adult bone marrow-derived mesenchymal stem cells. J Cell Physiol 2005, 203:398-409.
[6] Kafienah W, Mistry S, Williams C, Hollander A P: Nucleostemin is a marker of proliferating stromal stem cells in adult human bone marrow. Stem Cells 2006, 24:1113-20.
[7] Martin I, Vunjak-Novakovic G, Yang J, Langer R, Freed L E: Mammalian chondrocytes expanded in the presence of fibroblast growth factor 2 maintain the ability to differentiate and regenerate three-dimensional cartilaginous tissue. Exp Cell Res 1999, 253:681-8.
[8] Langdon W B: Performance of genetic programming optimised Bowtie2 on genome comparison and analytic testing (GCAT) benchmarks. BioData Min 2015, 8:1.

1. A method of determining, identifying or selecting a mesenchymal stem cell (MSC) having a high proliferative capacity, the method comprising:
(a) Obtaining data indicative of the level of expression of one or both of the following genes: GAS6 and GAS6-AS1 in the mesenchymal stem cell;
(b) Optionally calculating a ratio of the level of gene expression of GAS6-AS1:GAS6;
(c) Comparing the level of expression of one or both of the genes of step (a), and/or optionally the ratio of step (b), to a reference value;
(d) Determining, identifying or selecting the mesenchymal stem cell as having a high proliferative capacity if the level of expression of the GAS6 gene, GAS6-AS1 gene and/or the ratio of the level of gene expression of GAS6-AS1:GAS6, is below the reference value.

2. A method of distinguishing between a mesenchymal stem cell having a high proliferative capacity and a mesenchymal stem cell having a low proliferative capacity, the method comprising:
(a) Obtaining data indicative of the level of expression of one or both of the following genes: GAS6 and GAS6-AS1 in a mesenchymal stem cell;
(b) Optionally calculating a ratio of the level of gene expression of GAS6-AS1:GAS6;
(c) Comparing the level of expression of one or both of the genes of step (a), and/or optionally the ratio of step (b), to a reference value;
(d) Distinguishing the mesenchymal stem cell as (i) having a high proliferative capacity if the level of expression of the GAS6 gene, GAS6-AS1 gene and/or the ratio of the level of gene expression of GAS6-AS1:GAS6, is below the reference value, or (ii) having a low proliferative capacity if the level of expression of the GAS6 gene, GAS6-AS1 gene and/or the ratio of the level of gene expression of GAS6-AS1:GAS6, is above the reference value.

3. Use of the level of expression of one or both of the following genes GAS6 and GAS6-AS1, and/or the ratio of gene expression levels of GAS6-AS1:GAS6, as an indicator of the proliferative capacity of a mesenchymal stem cell.

4. An isolated mesenchymal stem cell, wherein the mesenchymal stem cell has a high proliferative capacity, wherein level of expression of the GAS6 gene, GAS6-AS1 gene and/or the ratio of the level of gene expression of GAS6-AS1:GAS6 in the mesenchymal stem cell is below a reference value.

5. The method of claims 1 or 2, the use of claim 3, or isolated mesenchymal stem cell of claim 4, wherein the mesenchymal stem cell expresses one or more of the following markers: CD90 and CD105, preferably wherein the mesenchymal stem cell is CD90+ and CD105+.

6. The method of claims 1, 2, or 5, the use of claims 3 or 5, or the isolated mesenchymal stem cell of claims 4 or 5, wherein the mesenchymal stem cell lacks expression of one or more of the following markers: CD34 and CD45, preferably wherein the mesenchymal stem cell is CD34− and CD45−.

7. The method of claims 1, 2, 5, or 6, the use of claims 3, 5 or 6, or the isolated mesenchymal stem cell of claims 4, 5 or 6 wherein the mesenchymal stem cell is derived from a tissue selected from: adipose tissue, bone marrow, umbilical cord tissue, blood, liver, dental pulp, and skin.

8. The method of claims 1, 2, or 5-7, the use of claims 3, or 5-7, or the isolated mesenchymal stem cell of claims 4 or 5-7 wherein the mesenchymal stem cell having a high proliferative capacity proliferates, or is capable of proliferating, at a faster rate, and/or proliferates, or is capable of proliferating, for a longer duration of time prior to senescence, when compared to the average proliferation rate and/or average proliferation duration in a typical or reference population of MSCs.

9. The method of claims 1, 2, or 5-8, the use of claims 3, or 5-8, or the isolated mesenchymal stem cell of claims 4 or 5-8 wherein the mesenchymal stem cell having a high proliferative capacity has a low cell doubling time, preferably wherein the mesenchymal stem cell having a high proliferative capacity has a cell doubling time of below 20 days, preferably below 10 days, preferably below 5 days.

10. The method of claim 9, the use of claim 9, or the isolated mesenchymal stem cell according to claim 9, wherein the mesenchymal stem cell having a high proliferative capacity maintains a cell doubling time of below 20 days, preferably below 10 days, preferably below 5 days for at least passage P1 to P10, preferably from passage P1 to P15.

11. The method of claims 1, 2, or 5-10, the use of claims 3, or 5-10, or the isolated mesenchymal stem cell of claims 4 or 5-10 wherein the mesenchymal stem cell having a high proliferative capacity has a long growth phase, preferably wherein mesenchymal stem cell having a high proliferative capacity has a growth phase of more than eighteen passages (P18), P19, P21, P22, P23, P24, P25, P26, P27, P28, P29, P30 and/or more passages, preferably of more than 20 passages (P20), preferably of more than twenty-two passages (P22), preferably more than twenty-four passages (P24).

12. The method of claims 1, 2, or 5-11, the use of claims 3, or 5-11, or the isolated mesenchymal stem cell of claims 4 or 5-11 wherein the reference value is the median level of expression of the GAS6 gene, the GAS6-AS1 gene, and/or the median ratio of GAS6-AS1:GAS6 gene expression in a reference population of MSCs.

13. The method of claims 1, 2, or 5-12, the use of claims 3, or 5-12, or the isolated mesenchymal stem cell of claims 4 or 5-12 wherein the reference value for GAS6 gene expression is 35, 36, 37, 38, 39, 40, 41, 42, 43, 44, 45, 46, 47, 48, 49, 50, 51, 52, 53, 54, 55, 56, 57, 58, 59, or 60 relative counts per million (CPM) at any passage number, preferably wherein the reference value for GAS6 gene expression is 42, 43, or 44 relative counts per million (CPM) at passage 15 (P15).

14. The method of claims 1, 2, or 5-13, the use of claims 3, or 5-13, or the isolated mesenchymal stem cell of claims 4 or 5-13 wherein the reference value for GAS6-AS1 gene expression is 10,000, 15,000, 20,000, 25,000, 30,000, 35,000, or 40,000 relative counts per million (CPM) at any passage number, preferably wherein the reference value for GAS6-AS1 gene expression is 20,000, 25,000, or 30,000 relative counts per million (CPM) at passage 15 (P15).

15. The method of claims 1, 2, or 5-14, the use of claims 3, or 5-14, or the isolated mesenchymal stem cell of claims 4 or 5-14 wherein the reference value for the ratio of gene expression of GAS6-AS1:GAS6 is 1:300, 1:350, 1:400, 1:450, 500, 1:550, or 1:600 at any passage number, preferably wherein the reference value for the ratio of gene expression of GAS6-AS1:GAS6 is 1:550, 1:600, or 1:650 at passage 15 (P15).

16. The method of claims 13-15, the use of claims 13-15, or the isolated mesenchymal stem cell according to any of claims 13-15 wherein the data indicative of the level of expression of one or both of the following genes: GAS6 and GAS6-AS is measured using RNA-seq.

17. A population of isolated mesenchymal stem cells according to any of claims 4-16.

18. A combination of the isolated mesenchymal stem cell according to any of claims 4-16, or the population of mesenchymal stem cells according to claim 17, and a therapeutic agent.

19. The combination according to claim 18 wherein the therapeutic agent is selected from a small molecule drug or pro-drug, a protein or peptide such as an enzyme or antibody, an oligonucleotide or a nucleic acid such as DNA or RNA (for example, mRNA, siRNA, miRNA), and a virus.

20. The combination according to claims 18 or 19, wherein the therapeutic agent is free, encapsulated, or attached to the mesenchymal stem cell.

21. The combination according to claim 18 or 19 wherein the therapeutic agent is produced by the mesenchymal stem cell, preferably wherein the mesenchymal stem cell comprises one or more nucleic acids encoding the therapeutic agent or a biosynthetic enzyme capable of synthesising the therapeutic agent.

22. A pharmaceutical composition comprising the population of mesenchymal stem cells according to claim 17, or the combination according to any of claims 18-21.

23. A population of mesenchymal cells according to claim 17 and/or a combination according to any of claims 18-21 and/or a pharmaceutical composition according to claim 22 for use as a medicament.

24. A population of mesenchymal cells according to claim 17 and/or a combination according to any of claims 18-21 and/or a pharmaceutical composition according to claim 22 for use in the treatment of a bone or soft tissue disease or injury, a liver disease or injury, a bowel disease or injury, a lung disease or injury, a skin disease or injury, a heart disease or injury, a kidney disease or injury, a uterus disease or injury, or any combination thereof.


SEQUENCE LISTING (4 sequences)

SEQ ID NO: 1 - DNA, 50525 bp, Homo sapiens
  source 1..50525: mol_type = genomic DNA; note = GAS6 gene
aagaaaacagaataaccaaacaactgcacactttgagaaagaagaacgtttgagtatttacactacctgagtatttacattacctgacttcaattcttgttatgaatctactatagtcaagacagtgtggtattaactttaaaaagagacaaacaaataaatgggacagaacagagtctacaaatagatttgcacatatattatcaattggttattgacaaaggtgcaaaggcaagtcagtggagaaagaatagtctttttaacagtgctgggatgattgcatttccctaggcacatacgtacagatacacactttgtttcatacttcagcatttataaaaactcattcaaaatggatcaccatcttaaatgtaaaactgaaatctacagaacttctgtaataaaatctttgtgaccttgagttaggcaaatgtgacaccaaaagcagaatccattaaagaaaataagctgcacttcaaaattaagaattctgatctttgaaagacactttaatttctttttatcttggagacagagtcttgctctattgtgcagaccaggcttaaactcctgggctcaagaaatcctcctgcctcagcctctcaagtagctgaagctacaggcacccactaccatgcctggctaaagacacatttaatagaatgaaaagacaagccgcagactggaaataaatatttgcaaaccacacatctgataatggacttgcttctagaatagttatagaactcttaaaactcaacaaacagctcaacaattggaaaaccaaacatcccaagtgtattagtcaattttcacgctgccgataaagacatacctgagactgggcagtttacaaaggaaagaggtttaactggactcacagttccacgtggctggggaagcctcacaatcatggcagaaggtaaggaggagcaagtcacgtcttacacagatggcagaaagcaaagacagagagcttgtgcaggggaattcctctttttaaaaccatcagatcttgtaagacttattcactatcatgacaacagcatgggaaagacttgcccccatgattcaattacctctcaccaggtccttcccataacatgtgggaagtcaagatgagatttcggtggggacacagccaaaccatatcaccaagtaaaggaaaagaaagaaagtaaaactgaaacagacatttcaccaaagaagatatctggatggcaataagtacaataaaatttgttcaacatcactagttatttgaaaaatgcaaatttaaactacaatgaaataccattatatacatattagagtgagtaaaattttttttttcagtggagtgtagggtttctttttttatttttatttttttattgatcattcttgggtgtttctcgcagagggggatttggcagggtcacaggacaatagtggagggaaggtcagcagataaacaagtgaacaaaggtctctggttttcctaggcagaggaccctgcggccttccgcagtgtttgtgtccctggatacttgagattagggagtggtgatgactcttaacaagcatgctgccttcaagcatctgtttaacaaagcacatcttgcaccgcccttaatccatttaaccctgagtggacacagcacatgtttcagagagcacagggttgggggtaaggtcacagatcaacaggatcccaaggcagaagaatttttcttagtacagaacaaaatgaaaagtctcccatgtctacctctttctacacagacacggcaaccatccgatttctcaatcttttccccacctttcccccctttctattccccaaaaccgccattgtcatcatggcccgttctcaatgagctgttgggtacacctcccagacggggtggtggtcgggcagagggcctcctcacttcccagtaggggcggccgggcagaggtgcccctcacctcccggacggggcggctggccgggcggggggctgacccccccacctccctcccggacggggcggctaaaaatttttaaagtgaccacagaaagtatgggtggggatgtggagcaactgggaccctcatgcattgatggtgaaaatgaaaaacagtataaccactttgaaaaacagtttaaaaagttaaacaaatacctactatgggatccagacactctgctcctagatatttccccaagagaaatggaagcacgtgtgcacacaatgtccacacgatggacacacacatgaatgcctacagcagctttatttgtgatagtaaaaatttagaagcaagtataatgtccctcaacaggtgaatgataaacaaattgtgttatatccatacagtggaatactactgagcaataaaaaggaatacacctgcaacaagatagatgattctgaaaataaatatgttaagtgaaaaaaacagacaaaaaagtgtacatacagtataattctttttatataaaagtctagaaaatacaaacaaatctatagtgatagaaagttggtcagcggtttttgcatagaaactgggatgtggggtagggtggggacatttaggagggaggaattaccaaagggcatgagaacacttttttgggtgatggatatgttccctaacttgattgtgatgatggcttcacaggtgtacacagctttccacctcatggccacagaccggttaaccatccatataaaacatccattgccctgggtccaacctcctgccccctgtggaggtgaactgtctacttcaccattgatatttcaggttgtatgatatagtgcctgacaactctccatttttggaacacatccaaattgtttcttattcattttccatttctgccttcgaacgaatttcctgcactggtaactaggtgaagaattttggagttatctggggtaatggagacatcctccattgcaccgcatatttgctttctgtattccccagttatagaaatcaaaacgagcagttgtatgtgaatgcattgtatcaaaccaagtctctagatgataggaggcattgtcattactaggatagtaactcagtagaaagagtgaattgagaatatgaggaatgagagtggggtccagactaaaaggaggccaggggtcctgaactgccccatccaactgagttcattctgatacctactgatctgaagcagcaggtccacgcccaggtgcagattgtcccagatgaacaggcagaggctagagatgctcattcctaaagggatcaaataattctgagaatggcaagaactccattaactctcttctccaaaactacaaaaatgtgcagaaaatacgaacaatttttaggagcttacactctttgaagtttatccatgggctgctggaacccttaggaaataatagctagaagtgcctcagacactgcctggcacacagcaggtgctcaatgaattacactgcctggcacatagtaggtgctcaatgaattgtctgatgaagtgattaaggagccttgtaaatactgattaaaacatgtatcaccccatccaatttttcaaaccagtgcttctcaagaacaagcaggaaaattaatgtaatattgttttgaagtagccgtggtggtttcggggcagaagcccaggggtactccttactccgcctgctccagtaactagctgacatggaagtgcctcgtcattctggttcagacacaggtgcagatagatgacttctctatcccactttaattctccttgaagatcattcccagggacattcatttgcttaaattaacatgctcggccaggcgcggtggctcacacctgtaatcccagcactttgggaggcccaggcgggcggatcacgaggtcaggagattgagatcatcctggctaacacggtgaaaccccgtctctactaaaaataaaaaaaatcagccgggcgtggtggcgggcgcctgtggtcccagctactcgggaggctgaggcaggagaatggcgtgaacccgggaggcggagcttgcagtgagcagagatcacgccactgcaccccagcctgggcaacatagcgagactccgtctgaaaacaaacaaacaacaacaacaaaaattcatgctcataatcccactttcaaaccagtgacctcaaagatttctagcagcaatcctattccattcttccaattacagaaagcaaagagaaactggatctggggatctaagcgttgagcctgctgagcagaacggccattcatttggggtgaggtgctgactcactgcccctctacattgcattttggggtgtggggatttccagagcaggattctccagagaggagtatccaagctccctcctcccgcagcagggggatgctgggctaactgcctggttcggtcccccgtgagtggtctgctgtgttttggttctgtttgtttgtgtgtgttttgcctatgactttgcgaaatattccaagacaagagccagaaagtcttggtctctgaagacaagcacaatgttgctttcagcttaaatatctactgggaaacctcaagatttttaatgtcatcattatgaaaaacagagattataaaaggaaaccgtcgcatttcactcgctggcccccagtgggattggatctgacctcagtgtatcttttctttggtaggttttaagtcttgacaaaatgtgcctaagaatgaagactccaaaaaggatgcagtagattcggggggagtgggggttggaaagaggcttaaatcttcacatgacacctggaatatggtcaaaatgtacctgctcaagttgaaccaggtattgcgtcctccttttaccccatcgctagtgaagacaaccaccgcctggggagacgctccttttccgaaacactcgagtcccgccgcgtcccggacgctccgctttccccgcgcccggatcctcccgggaacccgcgccccgcccgcgcgctggtcccgcccccgccgcctccccgcgcgcagccgcccgaacacgcccagaccgagcgcttgaggtgccgcagccgccgccgccgccgccgccgcgatgtgaccttcagggccgccaggacgggatgaccggagcctccgccccgcggcgcccgcggctcgcctcggcctcccgggcgctctgaccgcgcgtccccggcccgccatggccccttcgctctcgcccgggcccgccgccctgcgccgcgcgccgcagctgctgctgctgctgctggccgcggagtgcgcgcttggtgagtcccgcgggcccgagaccccgtccccgggcggcgggaggaggctccgggcgcggcttgacgcgtccccctctctccctccgggcagccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggtgagcagccggcgggcgggatgcgggggaacccctggcgcccgcgcgcctccaaccgctcccgagaggcagcgctgctgccgccccccagctccgcgcggcgaggcctggcctcagggtcccaggagccccggcccgccccgcgtcccccacgcccatcccccggcgaccccgggatccccgcgcaggccggtgagacgctgcggggctgtccgggaaacgctcgctgtcagacgcctccccgcggggaagccgggagtccgtttccaaagtgcgagagaaacacaatggttaaaacccaaaccgcttccccgcggccctccctctgctgggctggaggtgaggaacccccgggccgcgcctcgtcctcctgagaggggtggagagcagtcccctcccgaaagaggaattgaaagggccgagggcgggaactgcttgcggcagcaggtccaaaaataagattccaggaatcgagcggggccgcatgcgggaggggagggcggctccgcggccccgttctgccaggtgctctcctggaggcagcgggtgctttcaggagcccgacggtgctggccccggcacctcggttaggaatgtccttggagaaggcggggcgcccggggccgcctcccgcaggagccgccgtgtgaggacggcctggccactctatcctgcgggctcgggagcgggtgactttggaaagtccgagaaggtgtggattcccgttgacgtccctcccgccctgaagtcagggtgatttcatagcgaagttttccgttctgctggaatgttccttttaaagaattctcctggggtggaataaatgtcacccgggatgaaagagtggttctggatgaaccatggaggcctgtttccgcttacatttctagtggttgacttgacccctcttagatctgcctttctgaaagctctgaggctgtcagatggccactgtgcacttgtggacatagctcatccctgcagagatgacgcacctggggctgttgcccgggggctgcccacagccccacaggcccgtgccctccttcagctcagcctagcttggtcgctctccccggcctctctgttttcctgtttctttaatgatgaatcctggcctgtgctgctggctgaggcaggtgggcctgtgtgccctggtcgctcggagtcgccgcccagctgtggggttggtcgtgtggctggtgcagtgtcctccaacagcgtcctcacccggaacattctgcgtgcacagggactgtggcatggggcgtgctggtgatgggagttgagggcagcagggtcacccccaccccacagtacccgctccgcgcagccctgcaaggcctggccgcagcccctcctcgctgtcctccaggacctgcagggcccatgtcaatcagactggacgctgcctggatgtactgtgctgttcttcccaagtcagtcccctacctggttttccaattttaatttaataactttatttacaaaggtgtgaaatgaagataagaaaatgaaaacagcactcaggaccccatcactttaatacaactgcggttgcgtcacatggtttctgtgctggcatgttcatctcctcaccctcctgcatgtggctgagtggctttggtgcccgggaggcatccctcccatcctctcacagcctggccccagcagcctctgtcggcttcttttcagcgtcatctcagttcccagcgttctgccctccttcccagcccccatgccaaatgataacaacccgctttccctctggtttttggatctggccattagcttgctgtctgtttctgtttctgtttccggttcctgccggcaggtggatgctggggacagcctggttggggcaggggcaggggcagggtccagaggctggcttgagtcccagccggctccagcacaccaggcccacccctcccaggaccctagtctccactgcatcctctgccacactcttgggagaccccaggcccgaggggactgtccaagcacaacttcctgctaatgcatgaaaatcatccatgaacttctccctgtcaaaggccaggggctccttgtctttgaaatgttgctcactttacattttgtgcagtgcggctgattgaggaaaagggctgtggcctgagctcaagaggtgaccaggacctggccatggtgactgtcagagcatccttgggtccctggcctccccaggcccaggatcctgatgtgtggtcctccacgggtggttgtagggaaggtggtcccagccccctgccattttaggatgtgggaagtgcctggccgccgttagttaacacgcacctacccagccctggctgtttcccctgggcaggctcctacgagggagggaagggacagctggagccagctctccagtgtgctcaccttcatgtccacctgctaaactgctaacctgcccgaagtgtgtcccactgccccttgctggggcatcttcctggttgcgccttcttcccggaagcacccgctgtgcagcccttccagcaggcagatactaagcccggcacccctcccaggctccaggctcacttgtgtgctaacctctgtgcccacctggccacctgctcacctgcctgaagcgtgtcccactgccccaagctggggctgcttcctggttgtgagggaagtgcctgctctgcagacccccaccttccagcaggcggatactaagcccagcacccctcccaggctctggccctccccacacacctgtggtttcaagccctgttctgctggaagggacactcctcccacctccccagccctctctgctggagccgtgcccctcccccgacattcctccaggctgggccacccccacatgcctccttgctctccaccgcccagaccttctccttatcttgttttcctttttatattgttgttctccctccctggccttgcctctccctgctgggttccttgcccgagtttctaattcagagaagactgggtaactccccacagtgtctctgctaaagcttcccaggcccagtgactttttactaaatcctcgaaggccatcaggttccagggcctgctgaggtctccataatcaccccttgattccagaaatgatccgagactagagacttatcccagcccagccctactgcccaggtcctccggtgcctgcctgggagcctgtttgtgcactttgatccccggcctgcctgccggaaaatcatccaggctgtggttcctgatcccatcacctggctttatctgagatacaaagcacaatattcaccgagggctggtggccttccctgagggttggcctggattagcaacctgcacctgtacccgcacctgcacgacatgtggccaggcctgtgctccgaggccaggtttcccttcccccatctctttctttgtctccctctgtgtttctctctctgtcccagtctctctcttctccccactgctggtggtgaggaattctccgcatgggatgttggtgcatccgttttcaaacttgctggctcatcctgaactgcatgtgccctccaagcaccttttccctgaccttgtggattctttgggaacagctggggaaggggtaccctttatggaaatgaaaacccctctgagtccccacttgggaacagccacttggctgggacacaggcccagatgttgggatggcctaaagggaggtccctgttggaggggagggctgtacaatgtgggggcgcctggtggggaaggtgatggccaggctgagacctcccctgccctgccttgacagaggctgcagcacactcccacacacagacacatgcacacacattcacacatggacaccagcacagataggtgctgactcatggatacaggtgtgcagggacacacacacccctacctagacacatgcacacatgtacatatgttcccatagacacagatatacatgcacacacacaggcacacacatactaacagacatgtacatacgtatgcacacattcacatagatatacgtacacacacacactgacagacatgcacacacataaacaggcacacacacatactaacagacatgtacacatacatgcacacattcacatagacatacgtacacacagtcacacactgtccagacatgcacatacaaggcacacacatactaacagacatgtatacatacatgcacacattcacatagacatacagtcacacagacatgtacacatgcataatgacatgcacacacatactaacagacatgtacacatacatgcacacattcacatagatgtacctacacagtcacacagacatgcatacataggcacacacagacatgtacacatacgtacacagtcacacacacagacatgcacacacaaaggcacatactagcagacatgtacacatacatgcacacattcacatagacatatgtaagcacacagccacacactgacagacatgcacacacatacatgcacacattcacatagacatacgtacacacagtcacacactgtccagacatgcacatacaaggcacacatactaacagacatgtatacatacatgcacacattcatatagacatacagtcacacagacatgtacacacatgcatgacacgcacacacatactaacagacatgtacacatacatgcacacattcacatagacttacatagtcatgtacacacatgcataatgacacacatactaacagacatgtacacatacatgcacacattcacatagacatacatacagtcacagacatgtacacacatgcataatgacacgcacacacatactaacagacatgtacacatacatgcacacattcacatagatgtacatacacagtcacacacacagacatgcatacataggcacacacagacatgtacacatacagtcacaaacacacagacatgcatacataggcacacacagacatgtacacatacgtacacagtcacacacagacatgcacacacaaaggcacatactagcagacgtacacagacatgcacacattcacatagatgtacatagtcacacacagacatgcatacataggcacacagacatgcacacacaaaggcacatactagcagacatgtacacatgcacacattcacatagacatatgtaaacacacagtcacacacactgacagacatgcacacacatgcacacattcacatagacatacgtacacagtcatacactgacagacatgtacacacatgcataatatgcacacatgtacacacagtcacataaatagacacatatacacacacaggcacacatggatgcccagccacatgcatgcagacacacacagacacatgtgcaccctctcacacacttttctctttctacaactttggggtcagaagaccactgacagctgcagcaggatcactaaaaagtgacacaaggtcacggggctctgtggccccagcttcctcagacccccagggtctctccactcccagagccccctgcctgaggggccggagccctgtggccaaggcctttcctcctgggcccatcctgaggcctgcaggtggaccccggcccggcctcctctgagggccgtcttccgtgctggctccgcagccctctcctgtgcccgcacctgtgcagatgcttgttttcacccgggcgtctgcaggcacttggtgccaggctcctccggggctttctcagaggcagccgagtgccaggccctgcacacgacaccctcacccgagccggagagcttcacatctgtggaaaactctaattaaagacgttttgagaaacgctgaacgtctccctttgttatttaggaacaaaacactcttttaggaagcaagtgcttatggcttctttcttgagattctgagtaaaggggcgttaagcttgctagctggggcatgctgagcacttcccaggttatggctttaccccagagcgattgacaggcatccaggtttcctcatgcttcattttcccctgttttctaaggttaaaactgagagctgtttctccaatgcaaagctgcttgtttaagggtttcgtatttgatgttggaaatgctctggactcttccatggtttttatattccagtgatttcactgcaaacctggtgtgtacgccttgtaaattttgaccctggccaagtgaaaggttcattttaaaagttttaaaataaattttaaaggggagctggaagtggaataagttaagaacagtatcagttttgcctttcactctgacagaggcactctctgctctgactgacgggtgttgattaaaagtataatattcaaaagaaatagtaaagtgcattgggccttgttaatgttctactaattttctggggctccgttttcaaattataggtgaaatttttttccttccagtgttagtgcctatgataagaaaatacattttctccacatctgtccacaaacctgctagaccctgttttgtgactacctccccttggaacttggggctccatggaaaacacacagcttgacccagccagccctcagagagcctgggccggtcactgcagtggctggattctcagcataaaaaggccaaacgttcggccaggctgggactggctcccatccaggaccaactgtccacagtacagtaaggccactctctgagtcaacattgtttcttttctgaattgctaagttgaccgtccctgcagaagcagagagggctgcggtgtggcattcctgcccactccaaatgagggcagcccggacgcgggagctctcagggaggcgcagtgggctttgggtgtgtctggactgtctccaccacacctggaagatactgttcacttcctttttacacttgtgggaaatacatgacagtgtttacgggcaggctggctttccgtttatttcaaccctgacaattgaatagcagcagcagatgatgaaactgttcacttcccgatcactcccgttctcagatccatcacttagctcgtccgtcgctcagctcgtctgttagtctgaaagatgaatcgtgcacgggtcctcctgagagtgctgttctcggggtctccatgtgtgaagttgttacatcctcatgacaactgtgtaacagctgtcactgagccattttacagatgtggaaactgaggccctgagaggctgtggagggctacagtgtgtcctgggcatttgagccccacactctgtcattaactcctgtgctcgctgggactgggggagagcaggacgcggcagcacccccggccccactgtgtgctcctctgtgtgcaggcgactgtgagcatactggcacccatgtgagggctgagtggtgctggtgtgggcgcataaagcacagggcggctgcggggagctgggtgtgtgtcagtggctcggagcattagtctcgtgaacatggtctgccaggtgcgtcatgctgggctacagagcccatggctccaccataaatggtggttttgtgtttgaacttgagtgttgctgctcagtgaggccagaggaaggcaccaacctgcaggcctggccggtcctcacaacacggaaggcaaccagtccactagagagacatgtgtcccggccactggaggagtccaagtgacctttaagtctcaccagtcctcagagcaacccaggtgaatttggggcctgagcttggtcgtctggccgaagaccagctcaacttggataaagctggaggagaagcctgtccctcaggcagctgccagggcatctgtgatttctgcttgcgtgcactcttggcgactattggtggtggtcgcagtcagggcatctggttgcctttactgtccttacaccagctgggcagagctcaggctgggagcccagtgggagaggccggggcgtgactcccggcgcagaagcggcagtggcacaccggcgtcccaagcacagctgtccctgccaggcccctgcgcgttggatctttgggttgagtcatgctcgatggtgcagccatcctttcaagggaactgagctgtccagcgtcccatggagcagctccccgtggatccggagaggccgtaaataaacctccccatttgtaagccgggaacacacatgaactttgcaatcgtgtttgtcctgggatcatagaacgtggtggctggcaagacccaggcatcctcatcctgtgctgctgtgaacagaggccggtagcttattgagggaccagtcccagttcgctttctgtggggaagactctgatggaagcctaggccccaaactccaaggttagcacatcttcttggggggattgacacctccaaaggtgaacttgggtccaaggcacgtgtgtgcgacctgaagcatgggtgccgtgtgcaggagacgccctcaccggagatggggaccgagtccacacggctctaactgcagaatcactggccctgtgcccacgactattttattttgctgttttatatgatcacaaagtacactgtcttgtttttcccaaagcatttgcagtgagacatcccacttcctctccacggcagctgttgcgggcacgaggctgctcccaggacagcgatgccaccggggctggggtgggcacggaggaaccctcttccttcctcctccccgccctctgctcccccacagactggcctgtctttagtcagcactttcccaggctcccttctgcccaccacgtgcaaacagtggcgtgtcctgggcctctctccttgtagtgtttcttgtggccagcatccctcccatccaacagctgtgtggcttcctgactggcttctctcagggcccgcaaggatgctgccaaatggccagagacagtcactgctctgcacttcaaagcaggcagccctcggtggggcttgccctgaaagacagcgggccttcctcgatgagagcctgtgctggctcagggcctgtgggggctgcagctctgttccttccctgcgtccacgctcacatgctgaatgttgtgcaccacaggacaccctgctgggggctgtcgggagcatgctctgtgtttggggcagagacgggcagagtgagtctcagcattactgccgtgcagggttgagtctcagcatgacttccatgcagggggagtctcggcgtcactgccatgcggggcgagtctcggcgttaccgccgtgcagctgctggaagccactcgagctcctctgtctcgtttgccatgaagcccatgagagacactgatgtgtgtctcggggcttctgtgagaagagcttggttacccaccgggtcaggcaccacgtcggtgctgtgaggcatggagggtgctcacagctgtgcagtcagctacagaggcctttcccagcatccacatctgaccgggcagtcacaccaggatgagctcccaggaagagggctcccagcaagggagggcgcacctgggtgtgagcttggaggagggcagaggcacctgtcaccgctgaccctgggtgcagatgggcatgccgggtatactgactgtgctttcctggggcctgtctccctccaggtcagcctggggtcctctgggctgggggccatgcactgcctcctgtgggcagtgggatcagcacttccactttgctcagggctagagcaggctgctgcgggtgtgaagacttaggaaacgaattcggaaggaaggaaaggattttgcacttcccaagctagaagaacatgaatagagccgggctgccaggccaagggtctgtaagacggcggctcttcagagagtctgcaaatgctgagaaaggaaggctccccaagggcagttcctgtgggcgttactctgagttttctcatagaagtcaagttcttggaagacagcactatggtcaaagggttagtgaattcttgtgaccatttcccatggaaaatatcagagaaagttatggtgttgataaacagagtccctgtgctcagcagtctccacccacttgtcaagtaagataaagatttttggaaacaaaatttatggctgttatttgctaaagagagtaatacattcttagcacactctgaaaaataaccactatttgctgtatgaatgcagatgaaactcacacaaaaagatttatgcaggactgacatcgggtgtttgcagaaggttctccacgttcactgacctctgatcctcactgcagctctgcggggactgtggcccaaggatgagtgagcgtcaggtcgggccgtgcccttaatcccctggctgcccctcaccaccaggcagagaccttgctgggacccaaggagttaaagtgtattcagagatgaaggtgtttaggaaatgttctggaaccttccaaactgactttaggctgcgtgaggtgcttccagttcagtgcactttgggcccaaaagtgtttcctgtcttgttaatgcactgtgttgtgtgccatcaccgcatggaggttttggccatttttaaaaagtgaatgggccctcccagggtctagttggatcgagagaccactggaaaccctcaccaagctgcaggcaggtaggcaggggccccagagcaggcctaggggaagagtccccccggccccttggagggtctcttcacgtgctgggtgctgcctgattcctgcggtggcatggggaggtcaccccaccttgttctgaaccacagctctgggtgaggaagcctgtcaccaggttaaaggtcatttgggggttggggcgtgagaaccgcagtgccctgctggtagggttgccgcatgggtggccaggctacagcctggtccccgaggccgacccatcctgggaaccctgtgtgcagctccctgccatgcttgacctttgccctgtgggtggctttgagcgggggagacctgggtctggtggtgggcagggcaggggcggaggtggacttgggtctttgtgacactgcaccaattcccatggattctgccggcacccactgccctcctgagcagagcaaaggcagcagaggcggatgccagggtccggaggtggcatctactcatcagacactttctgtagttttataacaaatttggggcctcatgttagaccccagccagaccaggctcctgtcctggggctcagctccctccaagctgcttcaggttcagtttcctccatgcttacgaaaccgaagggcaacgggcacagtcagggaggcgtcagaggaggcaccggctccactgttcacaccgggaccttctgcagacggggctcccagcctcgaccagccccggaacccccagcacggggacgtccaatcttttggcttccgtgggccacattggaagaagaactgtcttgggccacacataaaattaacactaacagtagctgatgagctaaaaagaaggtctgtgcctaaagctcatgttttaagaaagtttacgaatttatgttgggctgcattcatggccatcctgggccactcacagttgcgccagagcctcgggagcacaggccgagcacctgccctgcttcacccagggacggggccctgcactgaccaggaggctgttgccctccaagtcgtccctgccgggaacagcaaagcacacaaacctccgagagacctaatggctgcatcctccgtgcagtgggcaccacgcttgccacctagaagagtgggtttggcagatgtcagttaataacaaggatgtaagttttaaaatcacgtgggggtttgtaaacttgaggaattacagcacaaacacttccatattattaaaagaaacagactgggagcattgattttgacttttcttccaagtacttatctatgatttaagtgaaactgagcattcagtgctctgtgggagtgaaaactgcaggcccttgcacttgcccactgattcattcattcacccatccacccactcattcattcaactatccatgcactcgctcatccatccatccactcattccttcatttattcatccatctccatccatccagtcacccattcacccactcactcatctatccactcactcacccacccattcactcatccattcacccacccactcacccacccacctattcattcattcatgtattcatctatctactcactctcattcacccattcattcattcatctgtccatccattcacctatccacccactcatccattcatgtactcactcatctacccactcactcattctttcattcattcatccattcactcacccatccacccactcattcattcactcatctggccactcactcatccacccactcactcattcattcatacactcactcatccatctattcactcattgttttattcattcatccatccatccatccactcacccatctatctacgcatttattcatttacccactcagtcatccgtccatcctcattcatttatccatccatctccattcatttactcatccacccactcattcattctttcatccacccactcatccatccactcattcatttgttcattcatccatctctatccactcatccattcattcattcccattcactcattcatccactgattcattcattcattcatctggttattcacccatccactcacttatccatccatgcatctgtctgctcactcatccatcctactcactaatctatccaccgattcactcatccatccattcattcattcatctgtgcatttatccatacatgcatctatccatccatctatccatctatcaatccatctgttcagtcattcatccattcactcatccatccatccacccactcattcatgcatccatctgcccatccactcatttattcacccatccattctttcacgcactcatccactgtccatctgtcatccatccatgtgtttggtgacggctcatgaggcctctgggggacagtcagcaccaggctcggtgctgggcaggtagatgttgtctttttcctcttgaagcttcagctaacagtgcagtggcttggggtttgctcacttatttttatctgtagctgttcaaataaaacagaagcagatccccggggaaaggatttccaggggccaaatggcgccatttccagcctgggaatcccagcttcttccccctgttgccctcacctaaccccagcacgccaacccattaccataggtcttgctcttcctaggtttagaagaaagagagtcagtgtgtacataaggagctcctggttctaaattccggcctcctgagtggtggcctctgctccatggggactcctgggggtgctgctctggagatatggctaatgcagggcttcaggccaaggtttatggacctgaacaagcagtagtgcagacaggggtgggagggttttgaggacaaagtgctctgtgtttggttctgcaggtcagggagcttatctgggtgcctttactcaaagccatttctgggtcttacatctttggctagaatgttctcaggacaatgtgaaatgtctttatctcaggagtgtataccagaatgttctcaggacaacgtgaaatgtctttctttcttgggagtgtgtaccagaatgttctcaggaaaatgtgaaatgtctctttttctttctctcaggagtgtatagcgtttttaggtttattcatcttagcttttctcttgctcatggtttaaccatctttcccctagtttgccggtggaattagcatatgctttctgcctccattactgtctcaaataatcaaacatgcaaattaggatacagtgcccagggctataaccctgtggtttcagaaaccccattcccataggtgtggcgaactgacaggagattggcatagtttttccatgaataatgcctttttgcaacaataaaacttggtattaatacagattacacattaagtagtatgactagtcttgattttgaaaaatagaacacacttgctgtaaagctgtggtaatgcaagcatttgtgtatagttaggccgtttgctcactagttcttagttgttttcaatccctggcttctaaacctgaaaggacaccttttgcatccctcgcaagtcttttatttcagagccagagcctcctgctttgagacaaagccccatcacttcccttctccttccaagcctagaaacctggtgccacttcgtgttgctccaggactccaccagggctcacttggtatggctgttcttgccttttcttggagtttgccaacccatgactaaaattggaagtgagagaaatgagggcaggcatgagggtgtgtgttccaggtctgcagccctcctggccttcagggcggccgccctcccaacagacacgtgagctgcacaccagctcaccctgacccttcctttctcttaagggagtaagagtttaagagcatcccatctttcctagaacacaactttgtgaaaacacatccaagcaggcctcaggagggtgttggcaaggctggggcggggcctccaggctcttcccacaaaaggtgtggcaaggccagcaagtgtgggagagaggtgggaaggaggcagaaaggccatgcagagccagcgcatgaagccagaggcgcgttgcagacggcgctgggtacctaccgcctctgtctgctactctgcaaaatgctaccagcgcctgctaatcctgaccttcatgcccccaggtgaatcccacaaccatcctgaagccgggtttcctcattcctgataatgggattaaaaagaccttcctgggtgttatttgtgaggatttcataaaatagtgtaaaaatgctgactcatacccagcatggagaggccgctgggactattctgccatgactgtggtctcttggtttagagcatttagggccaaactgctggcatggttgaccctaagctggagattggaggctgctttaaccaggctgtggccctagaatctgtgggccatggcaaaaggatcctgaagaagtttccggccggcctctgctggccaagtgacaggcaggtggttccaggactcgacagctcacttgtgtcgaatgtggacctcgtcagtgcgctttcatggataagaagacccttcattacagcctccatcctcacccagtaccaattttgtttcaccaatactataagtgcaaatcaatgcccgtgtgccaaaagaaaggtctttggggttttgagagacctttgtgtcaacagggaactaaggtctttgggttttagagacccttatgtcaacagggaactaagagtggggttttacttggaagcattcttgtcctcctgggcaggcctctcccctggacacacctggtagcacttcctacgcagctggaaatggccctgcctttgcttgttggtctcacagcagtcgtgccccggtcacttcccctcagttccccgggtggggcggccctctggctgccccgagagcagcctctgattaaccagctgatgcttatgttgttcagagaccctcgtagtgtgccggtcaatgcttgccttttctttttctttttccacaggattatttttacccaagatacttaggtaagtctcaattacttctctactctggttgtcgtagaggcatagttgggggtgcgtgtttcatgttggaggaatctcctcaccacgtaactcttggaaggaagattcttaatcacatggtgcacgtggaactgtccggaacatgcaggtcagaaacacaagtttctctctttattttataccacagctttattccgtgttagtggaacctcaggtgaatgctgttatctgcaaaccccttctctgagttgatgccaggctcagctccttgtcaggacgtgtaattgattttgtcctccggttttctgacctcagcactaatcacttctgaagtcattgaggaccccaaaggggtccatgttcatgtgggctgtattgactgatatttaccgtattcttaattaaaaccgaaaacactgaatagtgtttctgtttaatttgaagaacgggaatgccagacgttatctcagccatcagagcagctggtgcatgtggggcggcctctggagacccccactgtacacttgggaagggaggacagcaagaaagtgaaacccagagaccccgggtcagcccgtttaactgacaccatcttagagctctttgagagcatttcacttagaaggagagaaatgtattccagggtcttctttttaatgttgcaaagtgcattttagtaaatgtcctcttaaagggtccttccctgggtccatatctggaacaaacacagtgggtctggcactggcccagaaagcccaggcaccagcgaggactgagttctgaagcagggggtggccagcggtccacagcacacctgcaggaggccttccgctgttcatccgtgccgttctgcgcctggataagcaacagtaacccactgaagggccaggtcgagaggccccgcaccgttctgcacaacctcacgcttcgggttatccctggatgtgcatgtgccaggcctcgcctccccccgccgccctagcgggatgtctgctgtcaagctgtgttcagccagccagagagcatggaggggctttctccaaagcagagtggctttccagttaagcacagcgtttcccaatgggggtccactggaaaatgaagtttagcttttcgagtaccgtcaggaacttactgagcattcccggtgattctcccgtatggcaggaggagccccctcgaaacggcgggcggtgctttacgcacttgactgtgggtgcacaactccgctgagttggtggaggtggtttcctgtttgtcagggttggggctaacatgttttttattaacacaagctaagcggcattgtttctggaggatgatagcaccccaggtaagcagagtaactgcctctccagagcgtctcagaagcagaggctctgcagtccatccggtcttgtaaggatgactgacatccattccctttcttcctgcagactgcatcaacaagtatgggtctccgtacaccaaaaactcaggcttcgccacctgcgtgcaaagtaagtcggcctcctttgcgccttcctgggagcactgtgggtttgggcggctttaggcttggctgtggtttgtggagggccctgaatagttctgttttttaaggagctgtggtcccttgccgaggcctgctcccttttcgccaaactttcttttttaaacaagtaaattttgacgtttttagcaataggcaataaatgcacatggaacaaaatgtaaatgtttcacagagtgtgatgtgaaagcccatccccgcctctgagtcgtccagtcagacaccaccgtagttacagttccaagaatcttgtgcttagacatgtaaacatgtgtgagcggccttccgctcttcacacgcgtggcggcttagcacacacggtctgtcccgtgcctctgcaactctgtctgcacaggagaccctctcttagcgtgtctcaccttcacgctggcatttccagccggtgcatcatatgaacttagcccatgcatattggctgatgtccaggttatttccaatttccatcatttaaacaaaactggcgtgacatcctcatgcacctgttgcctcccacacgtctgagtgtctgcgggatgcgttcccaaggatggaatcgcagaggcagtcctcatgtttcctttggatacagatactgccaaattatcccagagccagccttagcctccgcccctgcaggggtgaatgaactgtttttttaaacttttgatctcttccaatgtaacaggcgagaaatgacatcagtcatttcaggtttttttttttttttttaactaatgagtaaagctgtccttcataggtttaaaaactgtgtatttcttttcctataaactgttgtttatattttttgcctgtttttctcaatttgtaggagtttgcacattagccattggtagtaaagttatcaaaaattttttcagcttgccacaagtcttttttattattttaaaattatcatgcaataagattgattgttttgggtgtcctgtatgaattttaacataccttcagcacaacccacacagctccccttgcactgggtctgtggtccgctggcgtcacgtgtgtctagttttagatgaaagtccagcttggtgagtccctgtgggcgctgagagcagaggcgtggcgggacggagtcatcctgggagaggcaggagtctcttcccaggcatggctttggagctggatcccagtcagcagaaggagtcagaggtggctcagtcccgctccccaagccccacgcactgtccgtgggcgtctctggcctcccggacttgtatcacagaccccagcccaagagacacaggcagaggaaatgctcagtgtaaacatgggaatgggagggtggccctgccctgtggcccggcctgccctgacctgcaaccatgcacagtagccaggtgccacgtagggcccaatgtctgcatttggggcagaggctcctgcgtgctcagaggagacaagcagatcctgtcacgcgtgttttatcttagtgattgctaaaccagtctcttcttttaaattctgtattcacacttgttatactctcttagttcctgggctttaggcttctccccaagattacaaaacagttacctgctgttaggtgttttcatctgtgacccgcccgtaatttcttttgacgtgatggatgaggtggggatctagactgcaggctcagttcctgccccaacaccacacatttgagtctgtctcaccccagtgcctcacctgctgccgtcctcatatgctgggtatttgggagattttctgccgggtttccacaggccgtggccacagctttagactcggcctcatgcacgcgggtgggtctcctatgccctcctcagtcttttttctggttgtcttgcttattttttaataaaactaaaccttaaaaatattcttaaaaacctaaaaaagccctttagatatttttaactgggcttgccattcatatatcggagactgtgtctgaacagtgttgcgctttcctaaccccggcaccgtcctgtccgtcccttggagcacttttcttcctgtgcgtcccgtgcttcctttgctcagttcacttctggctatttgcagcaaagaaggccgtccttttcaaggcgtcgcctcgcttccttgcatggatgaagaggagtgattgctacatgttcctttctgttcccgccgcccaatagagttatcttgttttggttatttcctttgtggcatcagcgtccccaagtgcggccagctggggagaggagggcaaggtcaagatccttgagtttgttccctgtgtgataggaagagaatctcttgctgtggctggtgccagaggcaacacatgcagtttctaacgggtccacctgccccaggccagaagctccggccactctccctgagccggccctcagaggctgccctgcctgtggcctggggctctcagcttctcctccagagcacgtggctggggcctctgatgttcgcgtgcctcaaaagatgcctggcggtgaagaaggtgctttggtggcctggccacagcaggcagccttgcactcccttgggaaggcgcctcccggaggatgcagctggggcagcctcacactctgttcctgttcattggttccctcgtgaaaattctctgacttggtgctcacggtgctgtcctggcttcagttcactggttatcggtcattgtgaaattaaagccatagccccataaagtccaaattcagccccagacaagcaggcccccagcgggccttgcatgggagcctgcggccctcaggctgtcccaggacccaggcggcacagtggacggagggaggaggcctgggaagagtgtaggctgcgttcccaggtgcccctgaagagcctctgcttccatttctttcctttcccacggacacacccagcttctccactgcgaacgcgcccgtcctgccatagcggccacttggtgcttggatttctcggctcctttgacccatgggtttccccaggaaggtcaggtccccacaggcggtggcccaggcagcggtcactgagccccggctaccgggatcccacccccgccacggggagtgtcccctcgcccttccctcgggcagacagggctctggtcctcgctcgtcaccacccagggaaaggccctggccgcccgcaccggcttcagattccagccacagccacagccgctccctcctgtccttcaacgcagtcatgggccaagctgagataaacccttcacctcctgaacaggcgctggcgcccagggcatgaccagccaggccaccgataccctaggcccagcttgcgggatggggatcagggaggtgggcgggggtcagggaggtggacggggtcagggaagtgggtgccctcgggggcctgcagaggtttctgtctccatcgctttccctgttgtgtggaggaaattccacagatatttgctgggcaccggctgactgtccacccctcaggtgtggttccgtccttgcatcctagaggtatcaatacattcccaacagggaaataaacaaatgagttgggggaggccccggggcaggtggcatgagtctggggaccgcttaagagctgggcacctggcaggagcaggcactgtccctcagcctcagggacagaggtctttccgaaaggcattccaggcaaagtcaagaccgggttcccagccctttgcatcacatgggcaggtgtccacttctgaaatggctttaatctctaaccaaacatcgtagcggcagcgcacagcatcccccagacatccctccatggtgggggcctctgaggaggtcaggttctggtgccggagggttgggccctgcacaggccgcctgccctgcacgtgtctgactccctcctgcccccagagcctcttcatctgaggccagggtggagaagcggatacgcccctcatttctctagttcaggccagacctccccgttccaggccctgctatgaccacatcgacgtctccagacgccccgcggacacagcacccaggtacagccgggcgacagcagtgtcagggatggggccccggccggctcctggcggctgtgctgatgcagaatccctggtcctggtcctcgtttcctcgtaactccgaaggccagccccagcccctgtgtgcttcagagtagaaagcgtttatagtgacctgctgaaaatagcatcttagaaacacttcgagacgcgagctccgtccacacccacaagtgttctgttgtcagtcatttgtgcaaaacaagcaaacggaggtgtagggaggaaactgtggggtgtactgaggaaactgaggcttatagaggaaactgtgaggcagacggaggaaactgaggtgtatggaggaaactgaagcatacagaggaaattgaggcgtatggaggaaactggggcgtatggaggaaactggggcgtatggaggaaactggggcgtacaaaggaaattgaggcgtatggaggaaactggggcgtatggaggaaactgaggcatatggaggaaactgtggggcgtacagagaaaattgaggcgtatggaggaaactgtggggtgtatggaagaaactggggggcgtatggaggaaattgaggcatatggaggaaactgtggggtgtatggaggaaactgggcatatggaggaaactgtggggcgtatggaagaaactgtggggcgtatggaggaaactgtggggcgtatggaggaaactgtggggcgtatggaggaaactggggggcgtatggaggaaactgggcatatggaggaaactgtggggtgtatggaggaaactgggcatatggaggaaactgtggggtgtatggaagaaactggggggcgtatggaggaaattgaggcatatggaggaaactgtggggtgtatggaggaaactgggcatatggaggaaactgtggggcgtatggaggaaactgtggggtgtatggaggaaaccggggcgtatggaggaaactggggcatacggaggaaactgtggggcgtatggaggaaattgaggcatatggaggaaactggggcatacggaggaaacgcgggtgtacggagcaaactgtgaggcatacggaggaaacgctgtgaggcatacggcgaagcgtcgcagaaagaagccagtgtggttgggcagcttccccaagaagcctgggactcgtagccagggcctctggtgagcgcggtgcctggacccgcctaggaggacaggggctgtgtctgtgccggccacaggcagtgactgtaagccctggatgagctggaggaactcagctttgtgaaccacagacccgcccgccgctcaccctggcaccgggcacccctgaccccggccaggtggggatggaagcggctcctgcactgacagccgagcccagaccctctggtggaaacgccacattctacagacctggtgggaaacgtcaccttgggagcctcaggaacagaagcgacacgctggcagtgagagcccatgcactgaagactcgacgtgcccccaccagggctgaccccttaggacgatgctggcccagtgccctagggccctgctggcccggcctgcacttttcccagggactggtccttattgaacttgtgtggaatcaatggaccggagccctctctcagccagcaggggggccctacccagggttccccacggagccctgtgtggctaagctgagcgtggctgagtcctcatgtggggctgagctgagtgtggaccgcgcccctgagcactgccatgctcagccccgagctgcttctgctctgccagcagccatgaaaggattctttgagctccgggtgtaattgtcgttctttctgccaacttcccggggctcaggcttgcgtaactgcactgtcgggggcttccctgcctgcctctgggacgtggtttgggtccgtgtcctggaagcgtgtttgtgagcagctcctcgtatgaatgtgtttgtgagcagcttctcggatgggggactgcttgggaagcagctgcactggcacgggttcccaccctcaggccctcacccgttcccctgacctctggccctgcaggagctgagcccaggtgaccaccctgagaaagggcagaacacacctggcctaaagccatcaagacacgtggagagggggcagctaagcagaaagaggggggcttcctggaggacccctctggcagtgcagcctgggcctggggtcctccccgccacacccagcagggagagttcaccagagatgggctaggggctggccagggaaggcagaatgttccgagtctgcctggaagcccccaggtcagctgggggttctgtgtgcctactccctgggaggtctgagctcccaggcctcacgccaggcagttcctgctcctttgccctcagcccttcccaccttgcgtccaggcctgctggcactccctcccagttgatctgagttttcctggagccggccccagggcccgccagcctattctgtcagccctaggccagcacagagggctcgggtgcccaggggctgcctcagcctgcgcccctcagagctcctccgggctggacccgagaccccacagccgccccgatctcagctgcgttgggcgcgcaggggtggggcagctgaacatgattttccactttgtgtccccaatcagacctgcctgaccagtgcacgcccaacccctgcgataggaaggggacccaagcctgccaggacctcatgggcaacttcttctgcctgtgtaaagctggctgggggggccggctctgcgacaaaggtacgtgaggctgaagacaaaggcggggtctccctccatctccgaccctgccctgatccccgctcctcactcccactccggggcacggccatcctcaggctgctctccgacagccccaaggaggctgcagccctgaggtatcctggagccccaggagaattgggaggagctgcagtctcctttctgggagcagggctggaaggaaaggaaacaccaagtgatgcctcagcacccactgattgaagggcggggggggaaatttcacctctgaccgtccttgagtgcagacctgtggtgtcagtggcacccactgattgaaaggcgggggggaaatttcacctctgaccgtccttgagtgcagacctgtggtgtcagtggcacccactgattgaaaggccggggggagatttcacctctgaccgtccttcagtgtagacctgtggtgtcagtgaagaccctggaggaagaatagctccagggcctggtggagacccatagtggagagcccagcaggccagcctgtcccctttggcaaatcctaccccacaggtggcctgggagggctttgctaaggagtggccagctctggcctggccttgtctgtcctcggcacccagaggtcactcctggcctgcagcctcctcctgcaagcacatgggcatccagggagctgagccctgtgggctgtgactgccagcagctccccaggctgagaccgcagctggggatcccccatgccctcccaccatctacctccacacgcaaggccaggaccaggggccatgaaacgctagggctgctgagtggggtgtgaggtccaagccctggggtccctctctgctccagggctgggctgtgcacctctaggatctctctctgctcgggggctgggctgtgcactcccaggctctctccctgctccagggctgggctgtgcacacccgcggtcccttcctcctgctccagtgctgggctgtgcacccccccggctccttccctgctccagtgctgggctgtgcacccccgggctccctccctgctccagggctgggctgtgcacccccggggtcccttcctcctgctgcggggctgggctgtgcacccccgggctccctccctgctccagggctgggctgtgcacccccggggtcccttcctcctgctccggggctgggctgtgcacccccgggctccctccctgctccagggctgggctgtgcacccccggggtcccctcctcctgctccggggctgggctgtgcacccctgggctccctccctgctccagggctgggctgtgcacccccggggtcccttcctcctgctccggggctaggctgtgacccctggtctccctccctgctccagggctgggctgtgcacctttgggatctctctctgctccagggctgggctgtgcacctctgggctccctccctgctccagggctgggctgtgcacctctgggatctcttcctaccgtagccaggggctgtgcacccttgggctccccttcaggcctgtgcttgttcccagatgtcaacgaatgcagccaggagaacgggggctgcctccagatctgccacaacaagccgggtagcttccactgttcctgccacagcggcttcgagctctcctctgatggcaggacctgccaaggtaaggccctggggtgaggcctctctcctctctcctctccccaccttctattttcggtcatgtctgctcttctgcaagcggctgctgctggctgggttcctgtggttctggatgggatggggcatcctgcagagactacacaagcccaggccagccgcagcacagggagctgctcggcaccacaccaggatgactcacccggcatctgagggccaggactcagggggccggcccagcccatgaggccacactgctggctgtagggcctcgtccattccccagagcctcacagagcctggaagtgctccaggcacttgtgtagtgagcttgtgggtcagggctccaggctagggtccccccgccgtccactctccccagcatccccagaagcaccccgttgttggtctccctctgtccactaaatatagccctgccctccccacttcctttcatatttcagtctggggttccctcagcccagggccagagctggggagccccaggacaccccagaaagtcactgtgaatgtgcgcgcccccgacgctgttccaggtgctgctgctgccactggccctttgccactttgcagggggagcaccttcctcctccctccccacgttgtatctggacatgcccaggcgtgtccctgagagtttctggcatagttcttagcgctgctgctgactgtccaggacaccgaactgcgtggacaggacagacagttgctcagggagaaacagccccaggatctggagagtgaatctgaccagacaggaggggccacaccccatcccccgatttcaaaaggcagctgctatttctgtccactttggtggttcccttccctgaccacccaggagttagccctgtgtgcagatgacgtgattcccatcttaacacataaacccatttccccgccgaggacagagcccctcccatcactgctctatccctgacctggaagacagcagcaccccaggtttaaagagaggtgaagactaggccgatcctgagagatgacgtctaaagcatctcccgtgggtccctgcaccagtcgggcactcccggcaaagtctaggctcacagttactgcccacagtgacagaggcattcccgtcctctcctcctcctccccatcttcctcttcctccccaccattccccctcctcctttccccactccccctcctcctctccccactccccctcctcctcccccccactccccctcctcctccccatcttcctcctcctcctcatcttcctcctcctctttcttctcctcctccccccactccccctcttcctccccatcttcttcctccccctcctcctcctcattcccctcttcttcctcctcattcccctcttcttcctcctcattccccctcctctttttccccctcctcctcccccatccttatttcccctctcctcctcctcccccacccctccccaccccctccctcacccctccccaccccctccctcacccctccccaccccctccctcacctcctcccccacccctccctcaccttctcctcttcctccctgcttctcctcctcctccccctccgtcgtcctccccctccgtcctcctccccctccgtcctcctccttcccttccccattctcctccccctccccctcctcctccccctcctcctcctccccctcctccccctcctctccatcctctccctaccccctcctcctcccctcctcccctccccctcctcttctccccctccccttctccccctcccccttctccccctcccccttctccccctcctcctcctcctcttcctccttcccctccccccttcctcctcccactcctactcccccttctcctcccccacctttctccgtgcccctgcctttgctctcctctggtgactaaagggctcttcatttcagggttgcccctcctaccccagggtgcttggccgtaaacagcatctgtcccacattagatgctcaagtcttaggattgtggtttaacccggtagtgaaaattgattgaaaccagtaaatgcttctctttggggttggggttttagtttcaaatgcccccggggggttactttttacggccccgtgtcctgtagcaccgtcatttaaatggaacagcacagcgtgcaccgccgccccccacccctccaccaagcagggcccttcccagctctccacctgctgggctgaagtcagccttcccagccgggccttgatcagaagcgtgcaccaacaccccgggagctgcccggtcaggggaggagggcagggaaatggggccagggcgcgctggccccacagagtctggatgcgacctctgggtggtgccctggccagtccctgcagccgcctgccccagccccgtctgagatgccgctgtgctgcggttggccggtttttttttgcttgcagacatagacgagtgcgcagactcggaggcctgcggggaggcgcgctgcaagaacctgcccggctcctactcctgcctctgtgacgagggctttgcgtacagctcccaggagaaggcttgccgaggtacccacgccacgcggtcaccctcgctttctctgacgcccaagtctagacacgggtgccagtcactgcccgagccggttgctaacaaccgggtgcttctggtgaaagaaagtctctttctccctgtttctatcagcctggtaatggcctgtgctaccaccatgcccgcacacacgtgagcacacacaggcacccatgccccttgtgcacatgaacgaaccgcacacatgcatatacaccgcatgtacacacgtgtgtacccttctccacctgcatagacggggcctctccccaaagggacggggagcagcaagcctccctctactgttctctctttccagccgccatggggagggccctgttggccgtagagcctctaagaggggccgtgcccccagtgatgggaacagtggggctgagtaggcagacagagagggtggggaagcccaggctgcctgcactgacctccatttcccaccacagctgtcggcccctctgtgctccgggggacacctcactgcacagcccagaccctgtgcttcccagacctgccttccacaccggcccggcagcgggggtgggtctggctcgttgggagcaccctcctgggacacctgcgacatttttattccctcaagaaagagcccgggcaggagcaggccgagggtgcaacctagctttccagtggggggggacgcgacccccaagccgcccccgttagaatctgcatgtaattccttgagcgcaccttgcgtgtggtttgagcaatcgcggtgtgatcccacggcgggacagcgtagaggcctccccggctcaccgccgcttccctctggctggcagatgtggacgagtgtctgcagggccgctgtgagcaggtctgcgtgaactccccagggagctacacctgccactgtgacgggcgtgggggcctcaagctgtcccaggacatggacacctgtgaggtaggcggcccctggccccgcgggcccttcacggtggttccgggggccctcgccggtgggggctttcgggagaaacacttccgtgaagacctggcctttgctaaggtgttggggatctccagggccatgtggaggtttgggatgagaaacgcttctgttccctggaagatgtttggttgtatttatagagtctctatggcaactcccagctcacagtggatcagcctggtgtggaccccagtgtgacactcacgggggcacggtccccactctgccgcgtgccagggagccaacgtgaccgacctgtcacaccggtgcctgtcacaccgacctgtcacaccggggcctgtcacaccggtgcctgtcacagcatgacctacctgtcacaccggggcctgtcacactggtgcctgtcacaccgacctgtcacaccggggcctatcacagcatgacctacctgtcacactggtgcctgtcacaccgacctgtcacaccggggcctatcacagcatgacctacctgtcacaccggggcctgtcacactggtgtctgtcacaccgacctgtcacaccggggcctgtcacagcatgacctacctgtcacaccggggcctgtcacactggtgcctgtcacaccgacctgtcacaccactgcctgtcacactgacttgtcacaccagtgtctcacaccgacctgtcacaacagtacctgtcacactgacctgtcacaccggtgcctgtcacaccactgcctgtcacactgacttgtcacaccagtgtctcacaccgacctgtcacaacagtacctgtcacaccgacctgtcacaccggtgcctgtcacaccactgcctgtcacactgacttgtcaccggtgtctgtcacaccgacctgtcacactggtgcctgtcacactggtgcctgtcacaccgacctgtcacaccggtgcctgtcacaccgacctgtcacactgacctgtcacaccggtaggaatgcagtacccacatgtggacgtttctgggcagggcggctcttgtctttcctcttcagcctgggcctgtgcctgggggttgatgagagtgagcatttatttaaaaagcaaaaccacaggtggaaagagtcaccaggacagcttctcggagtcgcagacctgggatgcagccgtggggctcttgggtctgggctgcgacgttcagggcttccagccagccctcgccttgaggttctttgcctcgctgcctcatgtactcatgcagagggtgtcggacccctgcgagatgtccagctcaccctggctgcccacggtgggcagggcaggcctggctcagccccagcccctccatcttccaggggtgtcagctcacaccggctttggttctgtcccccttcgggcagcgtggagaaaccacagcccagaacagggaactttccaggacagccatcttcaaggcatccatatctatttcataatagtgtatactttttaatgattctctgtaatttttgtatgcttgaaatatttcataatttaaaaataaagggtcaagggaaatgagcagggaaggagatgacggggacccccgagaagccctgtgggaagcggctgctgcaagcccgcccttcacctgggagtcccagtggggcaggtgtgacagcctctggggtctcagcagctagaggcggggtggccactcccgaggcacaggagggacagtggacccgctgcgcggccggggcgtggggctcaggggagcaggagtgaaggccacatccccgaccggcgtggcccccgtccgtggcaggacatcttgccgtgcgtgcccttcagcgtggccaagagtgtgaagtccttgtacctgggccggatgttcagtgggacccccgtgatccgactgcgcttcaagaggctgcagcccaccaggtgaggaggtgtccacaggaggcaacacaacttagaatggccaagacactggcttcacagggcccgggccagggttcggccagcagggaggggccgggcatctgcctgtgcccagtgctgactcatttctcctgtgggtgcccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacagtgagtgtgcggcagccaccccttccaggcctcacggggttctccctgagccagactcagctgtcactcgcttatgcaaccgttaggagatgagctctggggcctgcatctgctgcgtgccggcaggacggggacccctgccccgtttagttcatgctccgggagacggggacccctgccccgtttagttcatgctccgggggacggggacccctgccccgtttagttcatgctccgggggacggggacccctgccccgtttagttcatgctccgggggacggggacccctgccccgtttagttcatgctccgggggacggggacccctgccccgtttagttcatgctccgggggacggggacccctgccccgtttagttcatgctccgggggacggggacccctgccccgtttagttcatgctccgggggacggggacccctgccccgtttagttcatgctccgggggacggggacccctgccccgtttagttcatgctccgggggacggggacccctgccccgtttagttcatgctccgggggacggggacccctgccccgtttagttcatgctctgggggctcactgtggtgctctaggcctctctgctatttcatcacgtttcagcctggctccctcagaaaggggccttgagtgcaacctgtcagtttggtctccctgagtttggtcaccctggctcaggtagaacccgtggttctcagccccaggggaccctgggctgtgtctggaggccatgctgtttatcccgagcagagtggggaacagaagccagggctgcttgacgctacacgcagggtgccccaccaggggaggaggccgccactgaacccgcaccacagcccagagctcccgccgtacgtgtctcccgggccgtggccaggccaggcgccgcccgcccacccttccctgtaggacggcgtggacgctgcggtccctgagccctggctcccagggcctgctggtcagtgcggccatgcatgccaagggtccaggtgacctcacgggagctgccccttgccagagacgtcctgaccacctggaccccagccccatgggcaacctgaggggtcctggtcagacctgcacttgggcagctgtgggcagcggtaaaacgtgcccaccccacatccccatccgtggagcgagagggcctgtaagcaaccaggctcccgcccacagcgctgggcctccctcaccatactgggcttgcagaaggaaacgttgggaagaaaggtggaaagcacgtcttgcccttcagaaacattgtagacttgtatttccatcctgcaaagcaatgcctcgctccatttttcccaacctcttaaaggagcagtatctccaagagcaggaggctgttctcagcaaagctacagaaagcaaaggaagcagaagcagtttcagggatggagccccggggaggagggccccagggcaggtggcctgaggcgaggtcgcctgggggagcggtgttgcaggggatggagccatggggaggaggggcccggggcaggtggcctgaggcgaggtcgcctggagggagtggtgttgcaggggatggagccatggggaggaggggcccggggcaggtggcctgaggcgaggtcgcctgggggagcggtgttgcaggggatggagccatggggaggaggggcccggggcaggtggcctgaggcgaggtcacctgggggagcggtgttgcaggggatggagccacggggaggaggggcccggggcaggtggcctgaggcgaggtcacctgggggagtggtgttgcaggggatggagccacggggaggaggggcccggggcaggtggcctgaggcgaggtcgcctgggggagcggtgttgcaggggatggagccacggggaggaggggcccagggcaggtggcctgaggcgaggtcacctgggggagcggtgttgcagtgtagtcccgcccagtgactcacagcggccgcagcaccccaccaagacaggcccttccccagcaggggtacctcatgaggacccctccccaattccaccctctctagctgctgcccaaacccagctgacccgaatttgtaccatgtttttgtgactcagttcaaacccaaatacctgaaaggtgttttcacacaggaagaaggatattcttctgaagctcaggaatatagtgtatctagcattagactttacaggttttctatgaggaaaggttttggtgaccacattgatgcatcgagggccctcctggcccagatgatgagcagggactctctgaggttgggaccctcttggctcaggtgtggatcaggtggtctccttgaggtcaggtccctcttggctcaggtgaggatcaggtggtctccctgagattgggaccttcttggctcaggtgtggatcagggggtctccctgagattgggaccctcttggctcaggtgaggatcaggtggtctccccgaggtcaggtccctcttggcccaggtgaggatcaggtggcctccctgagattgggaccctcttggcccaggtgaggatcaggtggactccctgaggtcaggtccctcttggcccaggtgaggatcaggtggactccctgaggtcaggtccctcttggcccaggtgaggatcagatggtctccttgagatcaggtccctcatggctcaggtgaggatcaggtggtctctctgaggttgggaccctcttggctcaggtgtggatcaggtggtctctctgaagttgggaccctcttggcccaggtgaggatcaggtggtctccccgggtcaggtccctcttggcccaggtgaggatcatgtggtctccctgagattgagaccctcttggcccaggtgaggatcaggtggtgtccctgagattgggaccttcttggctcaggtgtggatcaggtggtctccctgaggtcaggtccctcttggccaggtgaggatcaggtggtctccctgagattgggaccctcttcgcccaggtgaggatcaggtggcctccctgagattgggaccctcttggcccaggtgaggatcatgtggtctgcctgagattgagaccctcttggcccaggtgaggatcaggtggtctccctgagatcgggaccctcttggctcaggggaggatcaggtggtctccctgaggtcgggaccctcttggcccaggtgaggatcaggtggcctccctgagatcgggaccctcttggcccaggtgaggatcatgtggtctgcctgagattgagaccctcttggcccaggtgaggatcaggtggtctccctgagatcgggaccctcttggctcaggggaggatcaggtggtctccctgaggtcgggaccctcttggctcaggggaggatcaggtggtctccctgagatcgggaccctcttggctcaggggaggatcaggtggtctccctgaggttgggaccctcttggcccaggtgaggatcaggtggcctccccgaggtcgggaccctcttggctcaggagaggatgagtgtggtttttctcagttgggagccccttcagaacgtgcactcccatcttttctcatggcgctgcctctcttcagatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctgtgagtactgtacctgtccctgtgtagacgcgccgtgtgctgggatcccgggaacggtgtcaggaagcctcagtcacaccctgccctgtgtttaccattaaccaggtgtgaggaggacctagaggaaggaaaggtttcctagagtttgacaaagtcgccgtacagctatgtcattaattttaccatctctttttatgagacctaagttttcatctgaattgataataaagttgtctatttttcaatggagataagacacaattaaattattattattttttttgagacggagtcttgctctgttgcccaggctggagtgcagtggcgcgatctcggctcactgcaagctccgcctcccgggttcacgccattctcctgcctcagcctcccaggcagctgggactacaggcgcccgccaccacgtccggctaatttttttgtatttttagtagagacggggtttcgccgtggtctcgatcttctgacctcgtgaactgcccacctcggcctcccaaagtgctgggattacaggcgtgagccaccgcgcccggccgaaattattctttaacagaacagtaaacttttttggacaatttccaagaacattttgggttttgtaactctgccgtgagtctcataaggggaaaacagccatgatgggcggtcagtgtgggccattgctccaggtcccgccttctctccccttcagcacagcacacgcccgtgcccccgacctgtgtctccagcccccacgtgctgacgtctaagccgtcatcctgggtattttcacttccacaccaggcaggagaaaggcacgcatgtcttcgcgtctttgggtgctgagtgtcggcagtgcctgtgctgaagccaccccttcagtgttgtgaggggcacctgctccccaggcccacagactgcctggtgccgggaccttcggtgttgtgaggggcacctgcttcccaggcccacagacagcctggtgccgggactttgcctggctggaccctcctcgtttcacagataagggggttgccctgatttgcccacagccatattgaatattgaataattcacatgagtgttggatggttgaacaatccatactctttgaccttagacccacatggccttttcagtcctttcctagctttaaaatgaacctttagttcttgagcatacaccagtggaaataggtattctgcagtgaggcgttgtgtaaagtgaagaaaccttctgtgaaatggccaccaccgatgggactggcccgtttctgccgtagagctgcacataccagggccgcgaaggcgccacccaccgactggggcatccggcatggggcttctcgctcccaggaagccacggagatttctctgcctttccagataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacagtaagtcttggctcttaccgttggggtggctgtggctgcaccttcagacaggcatacgctgaaagcaagcagacggcctcagatggcagcgtcaggacaagctgaggctcctccggttccggggcagggatgggtgggcggggtgggcggaggtgcgggcagaggcccacctctcaggatggccatgcaggtcactcagttcagagtcagccagtgaagaagaaatgagagagatgtgccgagaaggtgcctgcgaagccagtgccaggaggccggggagagaaggtgcctgcgaggccggcgccaggaggcgggggagagaaggtgcctgcgaggccggcgccaggaggccggggagagaaggtgcctgcgaggccggcgccgggaggccggggagagaaggtgcctgcgaggccagcgccgggaggccggggagagaaggtgcctgcgaggccggcgccgggaggccggggagagaaggtgcctgcgaggccggcgccgggaggccggggagagaaggtgcctgcgaggccggcgccgggaggctggggagagaaggtgcctgcgaggccagcgccgggaggccggggagagaaggtgcctgcgaggccggggaggctgggagccttacagggtcctttgcagaaagtccggagacagcacagccttaggactcgtccaggggcacagagaagctctcggtaacatggtggacaccgtggtcaccgatgacccggtgagacagctacaatgggaggtgtgtaaaaacagccgtgaagctgcctgggggacagtgaacctaggcctgaaaaagggaggacagggtggatgtgagctgctggcccagtcctgtgacgccacgctgccgccctccccagtgcaggctccgggggtgctgctgtgaggatagtgttactgctcggccccaccctgcctcacattcccaccccacgtgggccccactggggcaccacagccctgccctattcctggacaccgagccgtttatggtgaagcccccacacgcacagagggcaggcagcgccccctccgtcagatctcagggctgccggccctcaagcacccagcaggaaccccaggttcaaggggatccacgcggagcaaaggggagggtcctgccccgtcctgagccagatccagtgtcaccgtctccccccgggtgtcaaaagcgtgaccgtgtcaggaaaggcatctgagaaaagcgccatctgccactggctggactgactgtgcacacgcgttctccctcagtcttgatttttggacttccaaatctagagcatcataatgtttgacacgaaatcatgtcatatgaaatgcttaaatcctttttccacttaatatcatacacatttctcaggggtaaccttatgcaggcatcacaggggccatttccagtgactgccggccgcgtctgccggccctttttggtctatccggacagaagcagttacagctccatgcatttccagtgaatctcaaactataccagtaacgtctcttacaagacagaggagacttgctgggaatggtagtactggcctggctcactctgcagcgactctgctgggctcaggcttgcagatctccgggtctgtgcagcctccatgcaaggggccacgtggagccgggaaaccttcccggggagagcagaggccggtgcccttgtgaagccccgatggccaacatgtgcctagcctcctgtcaggagcttccttttttttttttttttttttttttgagacggagtttcgcctttgttgcccacactggagtacaatggcacgatcttggctccacctcccaggttcataccacaacctccgcctcccaggttcaagcgattctcctgcctcagcctcccgagtagatgggattacaggcatgcaccaccacacccagctaattttgcatttttagtagagatggggtttctccatgttggtcaggttggtctcgaactcccgaccttaggtgatccacccacctcagcctcccaaattgctaggattacaggcgtgagccaccacgcccgactgggagcttccattttaaagaacaccatcatctctcccttcacaggttatgggaagaccggcaaacaggagactttttgttgttactttttgattctgttgcaatatttttgttactttttctgattttgtgtgtttcggtgccataaatggttgttgagaactgatgtggcgggtaccagctagggacaggcaggacaggcgatgtgggtttcaggcgtacacagccccgtgggaaagagacagggaaaccagcacaattcactctgaccccaaccttggcctgcagtgcgaggcgcccagtcacaaatgtgtacacacagtgatttggggtcctttttcctaaaacagcttctttatcaggactttggaattctgggtgagatagaaacactgaaaacagggcggaagttttttcttctggcttcttagtccacggagggctcagcgtggagaggatatgccgtggcattctccctgggagaccacacatgttcccgacagctcagaccccagaccgcatgtgctcctgacagctcagaccccagaccgcgcgtgctcctgacagctcagaccccagaccgcaggtgctcccgacagctcagaccccagaccgcgggtgctcctgacagctcagaccccagaccgcgcgtgctcccgacagctcagaccccagaccgcgggtgctcctgacagctcagaccccagaccgcgcgtgctcccgacagctcagaccccagaccgcgggtgctcctgacagctcagaccccagaccacgcgtgctcccgacagctcagaccccagaccacggtgctcctgacagctcagaactcagaccgcgggtgctcctgacagctcagaccccagaccgcgcgtgctcctgacagctcagaccccagaccaccgtgctcatgacagctcctgtccgcatgctatcttaaactctcagcccccactgggccctgtgctgctacctgaaagctgcaggtgagatgcgaggcacgaggccacgcacgctctgaggaaggagctgtcaggcgctcctgaaacaagaggacatcggagggaggcagggacaggcctgcgtcccatggtgcacagcctgtggcccctcagacgggcctgtgtggcctggtggtcacctacagggcatttggaagtggggtactggtgtggccagggagaaacagcctgggggtccccggtgcctctgggctccgggccaagatctgaaccagtctgttgagttttccatcacgtcccagatccggggctgtctgcacagcttcccaccccggctgggactgcatctctcgaggccctgctccgacctcactgtggcctctccaccgtgcataccaccctataatgcaccgctttcattgcagtgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcaggtagggcctccgccacccagggcgtcctgaccggctcgacccacgggtacgtgggctgcactccctggcaggggacgaagggggcccgcggtgggatccccagggacccccaaagcctctgaccagaggtctgatgagccaagcccggcgtgggccacgcttcgggctgcgggggttgtttggacaccaggtcaccgccacacagctgagggggccggcgcctgcgagccacactcggctcacagtgtgggagcttttcccctgttcggttgggcagatggtggctcacagcaatctgaattagtctctggtgtttaagtcagatgatttcagcaacaaactcagttttccaatttccctggaaaagtcggatctgtgaccttggccggtcccccaacagcgtctttccgtgggggacaccgggtggagctgagtgcagctgccccttggacaaggtccccccacccccggtcctcacaataagtcagctgtcctgggctgggggactcatgctgttttccccacagaacaatcaagaggtgacagaaacagcttttattctctcgctcgtgtgctaaggagggagacatgggtcaaaccagagaggctcacgttctccaagtctgcccaccccggggccacagagccctcccgcttgctgggatacgacagacaggcttggttcatgcaaatgctcgttaccagggccagacccaatctcagcatctggaataaacagctgctgttatgtagcaacagccgacccctgggctgctgggctgtggtgccagcgtgagtgcccaccgggctgggaggccgccaccgcagggctgggtcctgggtccccatcagggccaccccagttcctggtcaagtgcagcctcggtcttgtgtgtgcctggtgcctggggctggggttgaccctctcacacccaacctgggggccacacttgttcccccacagcctcccaggctggcccctggctttacgtgcagagaagctgagccccaggtggggtgggggtgtcatccacagtcagcgagcatccagacccagggctgcagggaaccctggacggagacaggcgtgcggcgtaggggtggccagcagctgtgaggaccagcttggacctaacagctcacgtaggggtggccggcaggccctgaccacacctcggtctcctgcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccaggtaggtgctccctgctccgctcaacccgggtgaagagctccagggaggcccagcttggtcaccccagatctaacctggacaggctggggtttctggtagtgaatgcggaagaggacttgtgttttaagaggaaggagaagtccaggtgggtcgttagtggctattatggtgtctcggtgctggtcaggtcattggctgagactgagaaacacgtacgggggccggcattggcctgagtcaggccccacgaacctgcatcttcagggagcattcgtccacacccatcagaccagccccgggggagagggctgggcagacctcgggccaggctgcagttgggacccgcggctgtcaccgcagctgttagcagcgaacagagtgaccgccagcccctcccatgtccagcagggcgtcctgccgtgtcccatgggctacacagcacagaggctgggaccggggatgtccttgccatgagctgggagggtcagcggaaaggcacgtgggagccggcaagtcctcggctgcccgcaggttttcacttgcggcaaacggcaggatttctctcttggctgccagacacagactccacttagttttaataaacagagctttgaagaggatgagagcgagccaggaggcctgcagcctgaatgactcactgggcacagaagaagatcacgtttaaccgatgggactgtctgtgctcccagcggggaagggagggctggcgtctgcagagcccaggctggcagatgcctcctgcttgtctgccgggtctgcggccaaacgaacggggaccgagaagttagagaagaaagggaaaccctgccctttcggagggaacccggggctggccacaagcgctgccgggggtgggggggccaggcggggccggccacgtggtgagctaagatatgttgttctctccctgcggcccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaacaccgcgtggccgtcatctgctccccgcggggttctgcacacgtagcagcgtttgggatgagcggggctgccagggctccaggccgcaccctcagccgctccaaagatccctccctcttttctgctcccagcctggttgggcagccacggtctgctccccaggagcctctgactcctctggtagtggcggcaggggctggagcctcagtttacttgttctgtaaatgcggagaacgccctgctcggcccccaccacaggcagccagtcattgctgagtgaggagggcttggcacacaaatgcaggctgctggggcagcggcctctgagccaacctgggagggaccccgagctgggaccgagggccggaagacaccgaggcagcgcctgcacccctgtgccattcccgtccccaccctcctgcacagatccacttgcttccgggcctcaagtgtgttctgttgagaaagggaaactgctttctctgtgtcggtctggggtagagcttgccaggagcaaagccagccctgcttcccgcgtccccactggcagggtccccatgtccctcagcctcagcttccttgtctgcaaggggtggagcggtcactggtgctctctgaccccagcaggccacaagtctgcagggaaggtcagggcagccggaacgcggccaggcaggtgcctgaaatgcagtcgggaccctgcctgcctcctggaaggatgtcggagccgcagctggtgggggccacggagtcactgcttctccttggtcccaaggggacgaccaagactcatggcttgacgctcccgtgggacgcggccacctgtgctcgagcctctgggccaaacatgggtccccagggtggatgcggtcactgcaggggccacacgggggatcagggagggtttcagctctgaggcccaaaccaggattccactttaagaccaaacccccacccgtgctcggagctccctgggcaggagtgagacggggcagctcggagcagccccacctcccaccagtcagtcccagagctgcgcccaccgggcaggacaggctgtgcgtcggggcatctttgcctctgcctggctggggtcagcacttgacctagagaaaagtcgggtggagaagaaggatagaatccatgagaacccatccagggataaatcgccacaaatgaactgcaaagaggtttctagaaagaggcctgtggctttgaaggccacaggggaggccccgctggctggactgacaaggtggcatcggccgagtgggctcctgaggggcagcctcgggccgggaaggagcggcttccctctggcccccggcctgacggctgtggaatgggcagagcttctccaaggctcgggagccagcaacactcatgcccacctggagggaccctgcccgcctgggccgagctcaccccacggatggacggccagccaggcaccttgccaggggccatgcacagggggccccatcccctgccgtccagagccgcccagctcgtctacgctggagcctccaggccctggtggtccctgagggtccctggcaggcccaccctgtgtctctggggctcttgttgaccggtgccagttatgggctcctcgggccacatcccatgaccctgcgccatcgtgggcatgatgccacttgctctggacacgccgggcaagggcctcttgttctccccaaaatcttggcagcacctgggcagaagttctgttgattgagggctgcagcctgaattcaggacttcaacctagtctaagaggtattgcaggcaactttcgcttcggtaccgtgagaaaatagtgacgggggaggccctggcctcagagggcagacgtcaggctcctgccggctcccttcggaaacgtccatctgccgccagcggccccgcgtgtgtgtgacggctctcgccagcctcgcccagcagcacgtggaccttccgcaggcagatgcgctttctccactcgggccgggacagatggc

SEQ ID NO: 2 - DNA, 2037 bp, synthetic construct
  source 1..2037: mol_type = other DNA; note = GAS6 CDS
  CDS 1..2037: protein_id = 3; translation = MAPSLSPGPAALRRAPQLLLLLLAAECALAALLPAREATQFLRP RQRRAFQVFEEAKQGHLERECVEELCSREEAREVFENDPETDYFYPRYLDCINKYGSP YTKNSGFATCVQNLPDQCTPNPCDRKGTQACQDLMGNFFCLCKAGWGGRLCDKDVNEC SQENGGCLQICHNKPGSFHCSCHSGFELSSDGRTCQDIDECADSEACGEARCKNLPGS YSCLCDEGFAYSSQEKACRDVDECLQGRCEQVCVNSPGSYTCHCDGRGGLKLSQDMDT CEDILPCVPFSVAKSVKSLYLGRMFSGTPVIRLRFKRLQPTRLVAEFDFRTFDPEGIL LFAGGHQDSTWIVLALRAGRLELQLRYNGVGRVTSSGPVINHGMWQTISVEELARNLV IKVNRDAVMKIAVAGDLFQPERGLYHLNLTVGGIPFHEKDLVQPINPRLDGCMRSWNW LNGEDTTIQETVKVNTRMQCFSVTERGSFYPGSGFAFYSLDYMRTPLDVGTESTWEVE VVAHIRPAADTGVLFALWAPDLRAVPLSVALVDYHSTKKLKKQLVVLAVEHTALALME IKVCDGQEHVVTVSLRDGEATLEVDGTRGQSEVSAAQLQERLAVLERHLRSPVLTFAG GLPDVPVTSAPVTAFYRGCMTLEVNRRLLDLDEAAYKHSDITAHSCPPVEPAAA
atggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctag

SEQ ID NO: 3 - protein, 678 aa, synthetic construct
  source 1..678: note = GAS6 CDS translation
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 4 - RNA, 902 nt, Homo sapiens
  source 1..902: mol_type = other RNA; note = GAS6-AS1 gene
  ncRNA 1..902: ncRNA_class = lncRNA
tcattttctcttgctcctgccgtgtaagaagtgccttttgcctcccaccgtgattctgaggcctccccaaccatgtgcaattgcacaggcccaggctgaagaggaaagacaagagccgccctgcccagaaacgtccacatgtgggtactgcattcctaccggtgtgacagagggaagcggcggtgagccggggaggcctctacgctgtcccgccgtgggatcacaccgcgattgctcaaaccacacgcaaggtcctgccatcagaggagagctcgaagccgctgtggcaggaacagtggaagctacccggcttgttgtggcagatctggaggcagcccccgttctcctggctgcattcgttgacatctgggaacaagcacaggcctgaaggggagcccaagggtgcacagcccctggctacgcacacaggggctggggctggccttcggagttacgaggaaacgaggaccaggaccagggattctgcatcagcacagccgccaggagccggccggggccccatccctgacactgctgtcgcccggctgtacctgggtgctgtgtccgcggggcgtctggagacgtcgatgtggtcatagcagggcctggaacggggaggtctggcctgaactagagaaatgaggggcgtatccgcttctccaccctggcctcagatgaagaggctctgggggcaggagggagtcagacacgtgcagggcaggcggcctgtgcagggcccaaccctccggcaccagaacctgacctcctcagaggcccccaccatggagggatgtctgggggatgctgtgcgctgccgctacgatgtttggttagagattaaagccatttcagaagtggacacctgcccatgtgatgcaaagggctgggaacccggaaaaaaa